(19) United States
(12) Patent Application Publication
(10) Pub. No.: US 2021/0275479 A1
(43) Pub. Date: Sep. 9, 2021

(54) COMPOSITIONS AND METHODS FOR THE REDUCTION OR TREATMENT OF INSULIN RESISTANCE AND METABOLIC CONDITIONS

(71) Applicant: AXCELLA HEALTH INC., Cambridge, MA (US)

(72) Inventors: Michael Hamill, Wellesley, MA (US); Raffi Afeyan, Cambridge, MA (US); Chung-Wei Lee, Waban, MA (US); Harry Luithardt, Cambridge, MA (US); William Comb, Melrose, MA (US); Nadine Daou, Medford, MA (US); Svetlana Marukian, Watertown, MA (US)

(21) Appl. No.: 17/254,027

(22) PCT Filed: Jun. 19, 2019
(86) PCT No.: PCT/US2019/038037

§ 371 Date: Dec. 18, 2020

Related U.S. Application Data
(60) Provisional application No. 62/794,134, filed on Jan. 18, 2019.
(60) Provisional application No. 62/687,727, filed on Jun. 20, 2018.

(51) Int. Cl. A61K 31/198; A61K 38/05; A61K 38/06; A61K 31/4172; A61P 3/06; A61P 3/10

(52) U.S. Cl. CPC A61K 31/198; A61K 38/05; A61P 3/10; A61K 31/4172; A61P 3/06; A61K 38/06

(57) ABSTRACT

This disclosure provides compositions and methods for reducing or treating insulin resistance in a subject.

### BACKGROUND

Insulin resistance is a serious health problem characterized by the failure of insulin-sensitive cells to respond to the hormone, which normally results in the uptake and subsequent conversion of glucose into a storage form, such as glycogen. There are often abnormalities in lipid metabolism in individuals with insulin resistance, particularly high blood triglycerides and low high density lipoprotein. Various conditions and disorders are associated with insulin resistance, such as Type 2 diabetes, prediabetes, metabolic syndrome, obesity, hypertension, dyslipidemia, polycystic ovarian syndrome (PCOS), and hyperthyroidism. The primary treatment for insulin resistance is exercise and weight loss, while Metformin is one of the more commonly prescribed medications. However, Metformin is known to mask Vitamin B12 deficiency along with causing other side effects.

Given the limitations of available treatments, there is still a need for agents, e.g., dietary compositions and therapeutics that reduce insulin resistance in a subject.

### SUMMARY OF THE INVENTION

Provided herein is a composition including amino acid entities that is useful for improving insulin resistance or glucose tolerance in a subject, e.g., a subject with an insulin resistance condition or disorder. The composition can be used in a method of reducing and/or treating (e.g., reversing, reducing, ameliorating, or preventing) insulin resistance in a subject in need thereof (e.g, a human). The method can further include monitoring the subject for an improvement in one or more symptoms of insulin resistance after administration of the composition including amino acid entities.

In one aspect, the invention features a method for improving glucose tolerance in a subject, comprising administering to the subject in need thereof an effective amount of a composition (e.g., an Active Moiety) comprising, consisting essentially of, or consisting of:

a) a leucine amino acid entity,

b) a arginine amino acid entity,

c) glutamine amino acid entity; and

d) a N-acetylcysteine (NAC) entity;

thereby improving glucose tolerance in the subject.

In some embodiments, the glucose tolerance is not associated with a liver condition or disorder, type 2 diabetes, obesity, metabolic syndrome, or a high BMI.

In another aspect, the invention features a method for reducing insulin resistance in a subject, comprising administering to the subject in need thereof an effective amount of a composition (e.g., an Active Moiety) comprising, consisting essentially of, or consisting of:

a) a leucine amino acid entity,

b) a arginine amino acid entity,

c) glutamine amino acid entity; and

d) a N-acetylcysteine (NAC) entity;

thereby reducing the insulin resistance in the subject.

In another aspect, the invention features a method of treating insulin resistance in a subject in need thereof, comprising administering to the subject an effective amount of a composition (e.g., an Active Moiety) comprising, consisting essentially of, or consisting of:

a) a leucine amino acid entity,

b) a arginine amino acid entity,

c) glutamine amino acid entity; and

d) NAC entity;

thereby treating the insulin resistance in the subject.

In some embodiments, the insulin resistance is associated with a liver condition or disorder, type 2 diabetes, obesity, metabolic syndrome, or a high BMI.

In some embodiments, administration of the composition results in an improvement in a metabolic symptom in the subject, e.g., a metabolic symptom chosen from one, two, three, or more (e.g., all) of decreased free fatty acid, decreased lipid metabolism, increased insulin secretion, or impaired glucose tolerance.

In some embodiments, the composition is administered to a subject that has insulin resistance. In certain embodiments, the subject been diagnosed with insulin resistance.

In some embodiments, the composition is administered to a subject that has impaired glucose tolerance (e.g., hyperglycemia).

In some embodiments, the subject has prediabetes, type 2 diabetes, or metabolic syndrome (Syndrome X).

In some embodiments, the subject is overweight or obese.

In some embodiments, the subject has a cardiovascular condition or disorder. In certain embodiments, the subject has a cardiovascular condition or disorder chosen from: hypertension, dyslipidemia, atherosclerosis, or obstructive sleep apnea.

In some embodiments, the subject has an endocrine condition or disorder. In certain embodiments, the endocrine condition or disorder is chosen from: polycystic ovarian syndrome (PCOS), hyperthyroidism, Cushing's disease, Cushing's syndrome, acromegaly, or pheochromocytoma.

In some embodiments, the subject is pregnant.

In some embodiments, the subject has renal failure.

In some embodiments, the subject has a genetic condition or disorder. In certain embodiments, the subject has a genetic condition or disorder chosen from: Down's syndrome, Turner's syndrome, Klinefelter's syndrome, thalassaemia, haemochromatosis, lipodystrophy, progeria, Huntington's chorea, myotonic dystrophy, Friedrich's ataxia, Laurence-Moon-Biedl syndrome, a glycogen storage disease type I, a glycogen storage disease type III, or an inherited mitochondrial disorder.

In some embodiments, the subject has a cancer. In certain embodiments, the cancer is chosen from: colon cancer; endometrial cancer; pancreatic cancer; renal-cell cancer; or breast cancer.

In some embodiments, the subject has dementia. In certain embodiments, the dementia is chosen from: Alzheimer's disease or Lewy body dementia.

In some embodiments, the subject has a syndrome of severe insulin resistance (SSIR). In certain embodiments, the SSIR is associated with lipodystrophy.

In some embodiments, the subject has a genetic disorder of insulin resistance. In certain embodiments, the genetic disorder of insulin resistance is chosen from: Donohue Syndrome; Rabson-Mendenhall Syndrome; or Type A Insulin Resistance.

In some embodiments, the method further comprises determining the level of one, two, three, four, five, six, seven, eight, nine, ten, eleven, or more (e.g., all) of: (a) plasma insulin; (b) plasma glucose; (c) ACOX1; (d) Acta2; (e) plasma adiponectin; (f) alanine aminotransferase (ALT); (g) aspartate aminotransferase (AST); (h) caspase-cleaved keratin 18 fragments (e.g., M30 and M65); (g) FGF-21; (i) hydroxyproline; (j) IL-1β; or (k) IL-2.

In some embodiments, the method further comprises determining the level of one, two, three, four, five, six, seven, eight, nine, ten, eleven, twelve, or more (e.g. all) of: (a) body weight; (b) BMI; (c) HOMA-IR; (d) glucose utilization in hyperinsulinemic-euglycemic clamp; (e) 2-deoxy glucose uptake in ex vivo skeletal muscle or adipocyte prep; (f) 18FDG PET; (g) adiponectin; (h) retinol binding protein 4 (RBP4); (i) resistin; (j) insulin; (k) glucose; (1) leptin; or (m) adipocyte size.

In some embodiments, the composition (e.g., the Active Moiety) further comprises one or both of (e) an isoleucine amino acid entity or (f) a valine amino acid entity.

In some embodiments, the composition (e.g., the Active Moiety) further comprises one, two, three, or more (e.g., all) of: (g) a histidine amino acid entity, (h) a lysine amino acid entity, (i) a phenylalanine amino acid entity, or (j) a threonine amino acid entity.

In some embodiments, the total wt. % of (a)-(d), (a)-(f), or (a)-(j) is greater than the total wt. % of one, two, or three of other amino acid entity components, non-amino acid entity protein components (e.g., whey protein), or non-protein components in the composition (e.g., in dry form), e.g., (a)-(d), (a)-(f), or (a)-(j) is at least: 50 wt. %, 75 wt. %, or 90 wt. % of the total wt. of one or both of amino acid entity components or total components in the composition (e.g., in dry form).

In some embodiments, the composition (e.g., the Active Moiety) comprises a combination of 18 or fewer, 15 or fewer, or 10 or fewer amino acid entities, e.g., the combination comprising at least: 42 wt. %, 75 wt. %, or 90 wt. % of the total wt. of amino acid entity components or total components in the composition (e.g., in dry form).

In some embodiments, the composition does not comprise a peptide of more than 20 amino acid residues in length (e.g., whey protein), or if a peptide of more than 20 amino acid residues in length is present, the peptide is present at less than: 10 wt. %, 1 wt. %, 0.5 wt. %, 0.1 wt. %, 0.05 wt. %, 0.01 wt. %, 0.001 wt. %, or less of the total wt. of non-amino acid entity protein components or total components of the composition (e.g., in dry form).

In some embodiments, one, two, three, or more (e.g., all) of methionine, tryptophan, valine, or cysteine is absent from the composition, or if present, are present at less than: 10 wt. %, 1 wt. %, 0.5 wt. %, 0.1 wt. %, 0.05 wt. %, 0.01 wt. %, 0.001 wt. %, or less, e.g., of the total wt. of total components in the composition (e.g., in dry form). In some embodiments, one, two, three, or more (e.g., all) of methionine, tryptophan, valine, or cysteine, if present, are present in free form. In some embodiments, one, two, three, or more (e.g., all) of methionine, tryptophan, valine, or cysteine, if present, are present in salt form.

In some embodiments, methionine, tryptophan, valine, or cysteine, if present, may be present in an oligopeptide, polypeptide, or protein, with the proviso that the protein is not whey, casein, lactalbumin, or any other protein used as a nutritional supplement, medical food, or similar product, whether present as intact protein or protein hydrolysate.

In some embodiments, one, two, three, four, five, seven, eight, nine, or more (e.g., all) of (a)-(j) is selected from Table 1.

In some embodiments, the wt. ratio of the leucine amino acid entity, the arginine amino acid entity, the glutamine amino acid entity, and the NAC-amino acid entity is 1+/−20%:1.5+/−20%:2+/−20%:0.15+/−20%. In some embodiments, the wt. ratio of the leucine amino acid entity, the isoleucine amino acid entity, the valine amino acid entity, the arginine amino acid entity, the glutamine amino acid entity, and the NAC-amino acid entity is 1+/−20%:0.5+/−20%:0.5+/−20%:1.5+/−20%:2+/−20%:0.15+/−20%.

In some embodiments, the composition (e.g., the Active Moiety) comprises, consists essentially of, or consists of:

a) a leucine amino acid entity chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof;

b) an arginine amino acid entity chosen from: i) L-arginine or a salt thereof, ii) a dipeptide or salt thereof, or tripeptide or salt thereof, comprising L-arginine, iii) creatine or a salt thereof, or iv) a dipeptide or salt thereof, or tripeptide or salt thereof, comprising creatine;

c) the glutamine amino acid entity is L-glutamine or a salt thereof or a dipeptide or salt thereof, or tripeptide or salt thereof, comprising L-glutamine; and

d) the NAC entity is NAC or a salt thereof or a dipeptide or salt thereof comprising NAC.

In some embodiments, the composition (e.g., the Active Moiety) further comprises one or both of: e) L-isoleucine or a salt thereof or a dipeptide or salt thereof, or tripeptide or salt thereof, comprising L-isoleucine; or f) L-valine or a salt thereof or a dipeptide or salt thereof, or tripeptide or salt thereof, comprising L-valine.

In some embodiments, the composition (e.g., the Active Moiety) further comprises one, two, three, or more (e.g., all) of: (g) L-histidine or a salt thereof or a dipeptide or salt thereof, or tripeptide or salt thereof, comprising L-histidine; (h) L-lysine or a salt thereof or a dipeptide or salt thereof, or tripeptide or salt thereof, comprising L-lysine; (i) L-phenylalanine or a salt thereof or a dipeptide or salt thereof, or tripeptide or salt thereof, comprising L-phenylalanine; or (j) L-threonine or a salt thereof or a dipeptide or salt thereof, or tripeptide or salt thereof, comprising L-threonine.

In some embodiments, the composition (e.g., the Active Moiety) comprises, consists essentially of, or consists of:

a) the leucine amino acid entity is L-leucine or a salt thereof; b) the arginine amino acid entity is L-arginine or a salt thereof; c) the glutamine amino acid entity is L-glutamine or a salt thereof; and d) the NAC entity is NAC or a salt thereof.

In some embodiments, the composition (e.g., the Active Moiety) comprises, consists essentially of, or consists of:

a) the leucine amino acid entity is L-leucine or a salt thereof; b) the arginine amino acid entity is L-arginine or a salt thereof; c) the glutamine amino acid entity is L-glutamine or a salt thereof; d) the NAC entity is NAC or a salt thereof; e) the isoleucine amino acid entity is L-isoleucine or a salt thereof; and f) the valine amino acid entity is L-valine or a salt thereof.

In some embodiments, the composition (e.g., the Active Moiety) comprises, consists essentially of, or consists of:

a) the leucine amino acid entity is L-leucine or a salt thereof; b) the arginine amino acid entity is L-arginine or a salt thereof; c) the glutamine amino acid entity is L-glutamine or a salt thereof; d) the NAC entity is NAC or a salt thereof; e) the isoleucine amino acid entity is L-isoleucine or a salt thereof; f) the valine amino acid entity is L-valine or a salt thereof; (g) L-histidine or a salt thereof; (h) L-lysine or a salt thereof; (i) L-phenylalanine or a salt thereof; or (j) L-threonine or a salt thereof.

In some embodiments, the composition (e.g., the Active Moiety) is formulated with a pharmaceutically acceptable carrier.

In some embodiments, the composition (e.g., the Active Moiety) is formulated as a dietary composition.

### BRIEF DESCRIPTION OF THE DRAWINGS

FIGS. 1A-1B are graphs showing the effect of treatment with an amino acid composition (Amino Acid Composition A-1) on the NAFLD activity score, ballooning, and fibrosis in the STAM mouse model (FIG. 1A) and in the FATZO mouse model (FIG. 1B).

FIG. 2 is a schematic showing treatment regimens for administration of an amino acid composition to STAM and FATZO mice.

FIGS. 3A-3E are a series of graphs and images showing the effect of treating STAM and FATZO mice with an amino acid composition on the NAFLD activity score (NAS), steatosis, inflammation, and liver fibrosis as determined with histology.

FIGS. 4A-4B are images showing the levels of liver unsaturated fatty acids and acylcarnitines of STAM mice treated with the amino acid composition.

FIG. 5 is an image of a gene map of the liver gene expression pattern following treatment with the amino acid composition in STAM mice showing activation of ACOX1.

FIGS. 6A-6D are images of gene maps of the liver gene expression pattern following treatment with the amino acid composition in STAM mice showing upstream regulator activation of anti-inflammatory IL-10 (FIG. 6A); inhibition of pro-inflammatory NF-kB (FIG. 6B), interferons, IL-1b, and IL-2 (FIG. 6C); and suppression of the fibrogenic TGF-b signaling pathway (FIG. 6D).

FIG. 7 is a series of graphs showing MCP-1 and MIP-1 protein levels, which are the ligands of C—C chemokine receptor types 2 (CCR2) and 5 (CCR5), following treatment with the amino acid composition.

FIGS. 8A-8L are a series of microscopy images shown lipid accumulation in primary human hepatocytes following treatment with vehicle control (FIGS. 8A-8D), a LIVRQNAC amino acid composition (FIGS. 8E-8H), or free fatty acids and TNFα (FF+TNF; FIGS. 8I-8L).

FIG. 9 is a series of microscopy images showing liver histology (H&E stain or Sirius Red stain for collagen deposition) from FATZO mice after administration of the indicated amino acid compositions.

FIG. 10 is a series of microscopy images showing liver histology from FATZO mice after administration of the indicated amino acid compositions.

FIG. 11 is a series of graphs showing NAFLD activity scores (top left panel), Sirius Red staining (top right panel), steatosis levels (bottom left panel), inflammation levels (bottom middle panel), and ballooning (bottom right panel) observed in fixed liver tissues from FATZO mice after administration of the indicated amino acid compositions.

FIGS. 12A-12F are a series of graphs showing the effect of treating human subjects with an amino acid composition on levels of glucose (FIG. 12A), insulin (FIG. 12B), HOMA-IR (FIG. 12C), plasma adiponectin (FIG. 12D), liver fat and body weight (FIG. 12E), and plasma beta-hydroxybutyrate (FIG. 12F).

### DETAILED DESCRIPTION

Described herein, in part, is a composition (e.g., an Active Moiety) comprising amino acid entities and methods of reducing insulin resistance by administering an effective amount of the composition. The composition may be administered to treat or prevent insulin resistance in a subject in need thereof. The amino acid entities and relative amounts of the amino acid entities in the composition have been carefully selected, e.g., to reduce insulin resistance in a subject that requires the coordination of many biological, cellular, and molecular processes. The composition allows for multi-pathway beneficial effects on glucose and lipid metabolism to optimize modulation of signaling pathways involved in insulin resistance, glucose homeostasis, and lipid metabolism. In particular, the composition has been specifically tailored to improve glucose utilization, increase fatty acid oxidation, and increase insulin sensitivity.

In an example described in detail below, a composition of the invention improved insulin sensitivity as measured via fasting insulin (lowered), HOMA-IR (lowered) and adiponectin (increased), and increases fatty acid oxidation as measured via betahydroxybutyrate (increased).

### Definitions

Terms used in the claims and specification are defined as set forth below unless otherwise specified.

It must be noted that, as used in the specification and the appended claims, the singular forms “a,” “an” and “the” include plural referents unless the context clearly dictates otherwise.

As used herein, the term “amino acid entity” refers to a levo (L)-amino acid in free form or salt form (or both), the L-amino acid residue in a peptide smaller than 20 amino acid residues (e.g., oligopeptide, e.g., a dipeptide or a tripeptide), a derivative of the amino acid, a precursor of the amino acid, or a metabolite of the amino acid (see, e.g., Table 1). An amino acid entity includes a derivative of the amino acid, a precursor of the amino acid, a metabolite of the amino acid, or a salt form of the amino acid that is capable of effecting biological functionality of the free L-amino acid. An amino acid entity does not include a naturally occurring polypeptide or protein of greater than 20 amino acid residues, either in whole or modified form, e.g., hydrolyzed form.

Salts of amino acids include any ingestible salt. For pharmaceutical compositions, the salt form of an amino acid present in the composition (e.g., Active Moiety) should be a pharmaceutically acceptable salt. In a specific example, the salt form is the hydrochloride (HCl) salt form of the amino acid.

In some embodiments, the derivative of an amino acid entity comprises an amino acid ester (e.g., an alkyl ester, e.g., an ethyl ester or a methyl ester of an amino acid entity) or a keto-acid.

TABLE 1
Amino acid entities include amino adds, precursors, metabolites,
and derivatives of the compositions described herein.

| | Exemplary | | | |
|---|---|---|---|---|
| | Amino Acid | Precursors | Metabolites | Derivatives |
| Leucine | L-Leucine | Oxo- | HMB (beta- | N-Acetyl- |
| | | leucine | hydroxy-beta- | Leucine |
| | | | methybutyrate); | |
| | | | Oxo-leucine; | |
| | | | Isovaleryl-CoA | |
| Isoleucine | L-Isoleucine | 2-Oxo-3-methyl- | 2-Oxo-3-methyl- | N-Acetyl- |
| | | valerate; | valerate; | Isoleucine |
| | | | Methylbutyrl-CoA | |
| Valine | L-Valine | 2-Oxo- | Isobutyrl-CoA | N-Acetyl- |
| | | valerate | | Valine |
| Arginine | L-Arginine | Argininosuccinate; | Agmatine; | N-Acetyl- |
| | | Aspartate; Glutamate | Creatine | Arginine |
| Glutamine | L-Glutamine | Glutamate | Carbamoyl-P; | N-Acetyl- |
| | | | Glutamate | Glutamine |
| NAC | N-Acetylcysteine | Acetylserine; | Glutathione; | Cystine; |
| | | Cystathionine | Cystathionine; | Cysteamine |
| | | | Homocysteine; | |
| | | | Methionine | |
| Serine | L-Serine | Phosphoserine, | Glycine, | |
| | | P-hydroxypyruvate, | Tryptophan, | |
| | | L-Glycine | Acetylserine, | |
| | | | Cystathionine, | |
| | | | Phosphatidylserine | |
| Histidine | L-Histidine | Histidinol; | Carnosine; | N-Acetyl- |
| | | Histidinal; | Histamine; | Histidine |
| | | Ribose-5-phosphate | Urocanate | |
| Lysine | L-Lysine | Diaminopimelate; | Trimethyllysine; | N-Acetyl- |
| | | Aspartate | Saccharopine | Lysine |
| Phenylalanine | L-Phenylalanine | Phenylpyruvate | Tyrosine | N-Acetyl- |
| | | | | Phenylalanine |
| Threonine | L-Threonine | Homoserine; | Oxobutyrate | N-Acetyl- |
| | | O-PhosphoHomoserine | | Threonine |

“About” and “approximately” shall generally mean an acceptable degree of error for the quantity measured given the nature or precision of the measurements. Exemplary degrees of error are within 15 percent (%), typically, within 10%, and more typically, within 5% of a given value or range of values.

An “amino acid” refers to an organic compound having an amino group (—NH₂), a carboxylic acid group (—C(═O)OH), and a side chain bonded through a central carbon atom, and includes essential and non-essential amino acids and natural, non-proteinogenic, and unnatural amino acids.

As used herein, the term “Active Moiety” means a combination of four or more amino acid entities that, in aggregate, have the ability to have a physiological effect as described herein, e.g., an effect on insulin resistance. For example, an Active Moiety can rebalance a metabolic dysfunction in a subject suffering from a disease or disorder. An Active Moiety of the invention can contain other biologically active ingredients. In some examples, the Active Moiety comprises a defined combination of four or more amino acid entities, as set out in detail below. In other embodiments, the Active Moiety consists of a defined combination of amino acid entities, as set out in detail below.

The individual amino acid entities are present in the composition, e.g., Active Moiety, in various amounts or ratios, which can be presented as amount by weight (e.g., in grams), ratio by weight of amino acid entities to each other, amount by mole, amount by weight percent of the composition, amount by mole percent of the composition, caloric content, percent caloric contribution to the composition, etc. Generally this disclosure will provide grams of amino acid entity in a dosage form, weight percent of an amino acid entity relative to the weight of the composition, i.e., the weight of all the amino acid entities and any other biologically active ingredient present in the composition, or in ratios. In some embodiments, the composition, e.g., Active Moiety, is provided as a pharmaceutically acceptable preparation (e.g., a pharmaceutical product).

The term “effective amount” as used herein means an amount of an active of the invention in a composition of the invention, particularly a pharmaceutical composition of the invention, which is sufficient to reduce a symptom and/or improve a condition to be treated (e.g., provide a desired clinical response). The effective amount of an active for use in a composition will vary with the particular condition being treated, the severity of the condition, the duration of treatment, the nature of concurrent therapy, the particular active being employed, the particular pharmaceutically-acceptable excipient(s) and/or carrier(s) utilized, and like factors with the knowledge and expertise of the attending physician.

A “pharmaceutical composition” described herein comprises at least one “Active Moiety” and a pharmaceutically acceptable carrier or excipient. In some embodiments, the pharmaceutical composition is used as a therapeutic. Other compositions, which need not meet pharmaceutical standards (GMP; pharmaceutical grade components) can be used as a nutraceutical, a medical food, or as a supplement, these are termed “consumer health compositions”.

The term “pharmaceutically acceptable” as used herein, refers to amino acids, materials, excipients, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In a specific embodiment, “pharmaceutically acceptable” means free of detectable endotoxin or endotoxin levels are below levels acceptable in pharmaceutical products.

In a specific embodiment, “pharmaceutically acceptable” means a standard used by the pharmaceutical industry or by agencies or entities (e.g., government or trade agencies or entities) regulating the pharmaceutical industry to ensure one or more product quality parameters are within acceptable ranges for a medicine, pharmaceutical composition, treatment, or other therapeutic. A product quality parameter can be any parameter regulated by the pharmaceutical industry or by agencies or entities, e.g., government or trade agencies or entities, including but not limited to composition; composition uniformity; dosage; dosage uniformity; presence, absence, and/or level of contaminants or impurities; and level of sterility (e.g., the presence, absence and/or level of microbes). Exemplary government regulatory agencies include: Federal Drug Administration (FDA), European Medicines Agency (EMA), SwissMedic, China Food and Drug Administration (CFDA), or Japanese Pharmaceuticals and Medical Devices Agency (PMDA).

The term “pharmaceutically acceptable excipient” refers to an ingredient in a pharmaceutical formulation, other than an active, which is physiologically compatible. A pharmaceutically acceptable excipient can include, but is not limited to, a buffer, a sweetener, a dispersion enhancer, a flavoring agent, a bitterness masking agent, a natural coloring, an artificial coloring, a stabilizer, a solvent, or a preservative. In a specific embodiment, a pharmaceutically acceptable excipient includes one or both of citric acid or lecithin.

The term “non-amino acid entity protein component,” as used herein, refers to a peptide (e.g., a polypeptide or an oligopeptide), a fragment thereof, or a degraded peptide. Exemplary non-amino acid entity protein components include, but are not limited to, one or more of whey protein, egg white protein, soy protein, casein, hemp protein, pea protein, brown rice protein, or a fragment or degraded peptide thereof.

The term “non-protein component,” as used herein, refers to any component of a composition other than a protein component. Exemplary non-protein components can include, but are not limited to, a saccharide (e.g., a monosaccharide (e.g., dextrose, glucose, or fructose), a disaccharide, an oligosaccharide, or a polysaccharide); a lipid (e.g., a sulfur-containing lipid (e.g., alpha-lipoic acid), a long chain triglyceride, an omega 3 fatty acid (e.g., EPA, DHA, STA, DPA, or ALA), an omega 6 fatty acid (GLA, DGLA, or LA), a medium chain triglyceride, or a medium chain fatty acid); a vitamin (e.g., vitamin A, vitamin E, vitamin C, vitamin D, vitamin B6, vitamin B12, biotin, or pantothenic acid); a mineral (zinc, selenium, iron, copper, folate, phosphorous, potassium, manganese, chromium, calcium, or magnesium); or a sterol (e.g., cholesterol).

A composition, formulation or product is “therapeutic” if it provides a desired clinical effect. A desired clinical effect can be shown by lessening the progression of a disease and/or alleviating one or more symptoms of the disease.

A “unit dose” or “unit dosage” comprises the drug product or drug products in the form in which they are marketed for use, with a specific mixture of the active and inactive components (excipients), in a particular configuration (e.g, a capsule shell, for example), and apportioned into a particular dose (e.g., in multiple stick packs).

As used herein, the terms “treat,” “treating,” or “treatment” of insulin resistance refers to ameliorating insulin resistance (e.g., slowing, arresting, or reducing the development of insulin resistance or at least one of the clinical symptoms thereof); alleviating or ameliorating at least one physical parameter including those which may not be discernible by the patient; and/or preventing or delaying the onset or development or progression of insulin resistance.

### Compositions Comprising Amino Acid Entities (e.g., Active Moieties)

The composition of the invention as described herein (e.g., an Active Moiety) comprises amino acid entities, e.g., the amino acid entities shown in Table 1.

In certain embodiments, the leucine amino acid entity is chosen from L-leucine, β-hydroxy-β-methylbutyrate (HMB), oxo-leucine (α-ketoisocaproate (KIC)), isovaleryl-CoA, n-acetyl-leucine, or a combination thereof.

In certain embodiments, the arginine amino acid entity is chosen from L-arginine, creatine, argininosuccinate, aspartate, glutamate, agmatine, N-acetyl-arginine, or a combination thereof.

In certain embodiments, the glutamine amino acid entity is chosen from L-glutamine, glutamate, carbamoyl-P, glutamate, n-acetylglutamine, or a combination thereof.

In certain embodiments, the NAC amino acid entity is selected chosen from NAC, acetylserine, cystathionine, cystathionine, homocysteine, glutathione, or a combination thereof.

In certain embodiments, the isoleucine amino acid entity is chosen from L-isoleucine, 2-oxo-3-methyl-valerate (α-keto-beta-methylvaleric acid (KMV)), methylbutyrl-CoA, N-acetyl-isoleucine, or a combination thereof.

In certain embodiments, the valine amino acid entity is chosen from L-valine, 2-oxo-valerate (α-ketoisovalerate (KIV)), isobutyrl-CoA, N-acetyl-valine, or a combination thereof.

In certain embodiments, the histidine amino acid entity is chosen from L-histidine, histidinol, histidinal, ribose-5-phosphate, carnosine, histamine, urocanate, N-acetyl-histidine, or a combination thereof.

In certain embodiments, the lysine amino acid entity is chosen from L-lysine, diaminopimelate, aspartate, trimethyllysine, saccharopine, N-acetyl-lysine, or a combination thereof.

In certain embodiments, the phenylalanine amino acid entity is chosen from L-phenylalanine, phenylpyruvate, tyrosine, N-acetyl-phenylalanine, or a combination thereof.

In certain embodiments, the threonine amino acid entity is chosen from L-threonine, homoserine, O-phosphohomoserine, oxobutyrate, N-acetyl-threonine, or a combination thereof.

In certain embodiments, the serine amino acid entity is chosen from L-serine, phosphoserine, p-hydroxypyruvate, glycine, acetylserine, cystathionine, phosphatidylserine, or a combination thereof. In some embodiments, the serine amino acid entity is chosen from L-serine or L-glycine. In one embodiment, the serine amino acid entity is L-serine. In another embodiment, the serine amino acid entity is L-glycine. In another embodiment, the serine amino acid entity is L-glycine and L-serine (e.g., L-glycine and L-serine at a wt. ratio of 1:1).

The composition described herein can further comprise one, two, three, or more (e.g., all) or more of L-serine, L-glycine, creatine, or glutathione. In some embodiments, the composition comprises a leucine amino acid entity, an isoleucine amino acid entity, an valine amino acid entity, an arginine amino acid entity, a glutamine amino acid entity (e.g., L-glutamine or a salt thereof), a NAC-entity, and L-serine. In some embodiments, the composition comprises a leucine amino acid entity, an isoleucine amino acid entity, an valine amino acid entity, an arginine amino acid entity, a glutamine amino acid entity (e.g., L-glutamine or a salt thereof), a NAC-entity, and

L-glycine. In some embodiments, the composition comprises a leucine amino acid entity, an isoleucine amino acid entity, an valine amino acid entity, an arginine amino acid entity, a glutamine amino acid entity (e.g., L-glutamine or a salt thereof), a NAC-entity, L-glycine, and L-serine.

In some embodiments, one, two, three, four, five, seven, eight, nine, or more (e.g., all) of (a)-(j) are in free amino acid form in the composition, e.g., at least: 42 wt. %, 75 wt. %, 90 wt. %, or more of the total wt. of amino acid entity components or total components is one, two, three, four, five, seven, eight, nine, or more (e.g., all) of (a)-(j) in free amino acid form in the composition (e.g., in dry form).

In some embodiments, one, two, three, four, five, seven, eight, nine, or more (e.g., all) of (a)-(j) is in salt form in the composition, e.g., at least: 0.01 wt. %, 0.1 wt. %, 0.5 wt. %, 1 wt. %, 5 wt. %, or 10 wt. %, or more of the total wt. of amino acid entity components or total components is one, two, three, four, five, seven, eight, nine, or more (e.g., all) of (a)-(j) in salt form in the composition.

In some embodiments, one, two, three, four, five, seven, eight, nine, or more (e.g., all) of (a)-(j) is provided as part of a dipeptide or tripeptide, e.g., in an amount of at least: 0.01 wt. %, 0.1 wt. %, 0.5 wt. %, 1 wt. %, 5 wt. %, or 10 wt. %, or more of amino acid entity components or total components of the composition.

In some embodiments, the composition (e.g., the Active Moiety) is capable of enhancing fatty acid oxidation, e.g., one or both of reducing levels of unsaturated fatty acids or increasing levels of acylcarnitine (e.g., in a STAM mouse model or a FATZO mouse model). In certain embodiments, the reduction in levels of unsaturated fatty acids is at least 25%, 40%, or 50% of the level of change shown in Table 53, e.g., measured as described in Example 3. In certain embodiments, the increase in levels of acylcarnitine is at least 25%, 40%, or 50% of the level of change shown in Table 53, e.g., measured as described in Example 3.

In certain embodiments, the composition (e.g., the Active Moiety) is capable of decreasing, or decreases, triglyceride levels in hepatocytes, e.g., by at least 15%, 20%, or 30% e.g., as assessed using a colorimetric assay, e.g., as described in Example 7, e.g., relative to a reference composition (e.g., a lower concentration of the composition, a vehicle control (PBS), a single amino acid entity, or combination of amino acid entities).

i. Amounts

The composition (e.g., the Active Moiety) can include 0.5 g+/−20% to 10 g+/−20% of a leucine amino acid entity, 1 g+/−20% to 15 g+/−20% of an arginine amino acid entity, 0.5 g+/−20% to 20 g+/−20% of a glutamine amino acid entity, and 0.1 g+/−20% to 5 g+/−20% of a NAC-entity.

An exemplary composition can include 1 g of a leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.15 g of a NAC-entity (e.g., g/packet as shown in Table 3).

TABLE 3
Exemplary composition including the form L-arginine (R) or L-arginine HCl (R HCl)).

| | wt. ratio | wt. ratio | wt. % | wt. % | g/packet | g/packet | g dose #1 | g dose #1 | | g dose#2 | g dose#2 | |
|---|---|---|---|---|---|---|---|---|---|---|---|---|
| Amino acid | (R) | (R HCl) | (R) | (R HCl) | (R) | (R HCl) | (R) | (R HCl) | | (R) | (R HCl) | |
| Leucine | 1 | 1 | 17.70 | 16.78 | 1.00 g | 1.00 g | 2 | 2 | g | 4 | 4 | g |
| Isoleucine | 0.5 | 0.5 | 8.85 | 8.39 | 0.50 g | 0.50 g | 1 | 1 | g | 2 | 2 | g |
| Valine | 0.5 | 0.5 | 8.85 | 8.39 | 0.50 g | 0.50 g | 1 | 1 | g | 2 | 2 | g |
| Arginine | 1.5 | 1.81 | 26.55 | 30.37 | 1.5 g | 1.81 g | 3 | 3.62 | g | 6 | 7.24 | g |
| Glutamine | 2 | 2 | 35.4 | 33.56 | 2.00 g | 2.00 g | 4 | 4 | g | 8 | 8 | g |
| NAC | 0.15 | 0.15 | 2.65 | 2.52 | 0.15 g | 0.15 g | 0.3 | 0.3 | g | 0.6 | 0.6 | g |
| Total AAs | | | | | 5.65 g | 5.96 g | 11.3 g | ~12 | g | 2.2.6 g | ~24 | g |

In some embodiments, the composition (e.g., the Active Moiety) includes 1 g+/−20% of a leucine amino acid entity, 0.5 g+/−20% of an isoleucine amino acid entity, 0.5+/−20% g of a valine amino acid entity, 1.5 g+/−20% of an arginine amino acid entity, 2 g+/−20% of a glutamine amino acid entity, and 0.15 g+/−20% of a NAC-entity. In some embodiments, the composition includes 1 g+/−15% of a leucine amino acid entity, 0.5 g+/−15% of an isoleucine amino acid entity, 0.5+/−15% g of a valine amino acid entity, 1.5 g+/−15% of an arginine amino acid entity, 2 g+/−15% of a glutamine amino acid entity, and 0.15 g+/−15% of a NAC-entity. In some embodiments, the composition includes 1 g+/−10% of a leucine amino acid entity, 0.5 g+/−10% of an isoleucine amino acid entity, 0.5+/−10% g of a valine amino acid entity, 1.5 g+/−10% of an arginine amino acid entity, 2 g+/−10% of a glutamine amino acid entity, and 0.15 g+/−10% of a NAC-entity. In some embodiments, the composition includes 1 g+/−5% of a leucine amino acid entity, 0.5 g+/−5% of an isoleucine amino acid entity, 0.5+/−5% g of a valine amino acid entity, 1.5 g+/−5% of an arginine amino acid entity, 2 g+/−5% of a glutamine amino acid entity, and 0.15 g+/−5% of a NAC-entity. In some embodiments, the composition includes 1 g of a leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.15 g of a NAC-entity.

In some embodiments, the composition (e.g., the Active Moiety) includes 1 g+/−20% of a leucine amino acid entity, 0.5 g+/−20% of an isoleucine amino acid entity, 0.5+/−20% g of a valine amino acid entity, 1.5 g+/−20% of an arginine amino acid entity, 2 g+/−20% of a glutamine amino acid entity, and 0.3 g+/−20% of a NAC-entity. In some embodiments, the composition includes 1 g+/−15% of a leucine amino acid entity, 0.5 g+/−15% of an isoleucine amino acid entity, 0.5+/−15% g of a valine amino acid entity, 1.5 g+/−15% of an arginine amino acid entity, 2 g+/−15% of a glutamine amino acid entity, and 0.3 g+/−15% of a NAC-entity. In some embodiments, the composition includes 1 g+/−10% of a leucine amino acid entity, 0.5 g+/−10% of an isoleucine amino acid entity, 0.5+/−10% g of a valine amino acid entity, 1.5 g+/−10% of an arginine amino acid entity, 2 g+/−10% of a glutamine amino acid entity, and 0.3 g+/−10% of a NAC-entity. In some embodiments, the composition includes 1 g+/−5% of a leucine amino acid entity, 0.5 g+/−5% of an isoleucine amino acid entity, 0.5+/−5% g of a valine amino acid entity, 1.5 g+/−5% of an arginine amino acid entity, 2 g+/−5% of a glutamine amino acid entity, and 0.3 g+/−5% of a NAC-entity. In some embodiments, the composition includes 1 g of a leucine amino acid entity, 0.5 g of an isoleucine amino acid entity, 0.5 g of a valine amino acid entity, 1.5 g or 1.81 g of an arginine amino acid entity, 2 g of a glutamine amino acid entity, and 0.3 g of a NAC-entity.

An exemplary composition can include 1 g of a leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.15 g of a NAC-entity (e.g., g/packet as shown in Table 4).

TABLE 4
Exemplary composition including the form L-arginine (R) or L-arginine HCl (R HCl)).

| | wt. ratio | wt. ratio | wt. % | wt. % | g/packet | g/packet | g dose #1 | g dose #1 | | g dose#2 | g dose#2 | |
|---|---|---|---|---|---|---|---|---|---|---|---|---|
| Amino acid | (R) | (R HCl) | (R) | (R HCl) | (R) | (R HCl) | (R) | (R HCl) | | (R) | (R HCl) | |
| Leucine | 1 | 1 | 20.41 | 19.78 | 1.00 g | 1.00 g | 2 | 2 | g | 4 | 4 | g |
| Isoleucine | 0.5 | 0.5 | 10.20 | 9.89 | 0.50 g | 0.50 g | 1 | 1 | g | 2 | 2 | g |
| Valine | 0.5 | 0.5 | 10.20 | 9.89 | 0.50 g | 0.50 g | 1 | 1 | g | 2 | 2 | g |
| Arginine | 0.75 | 0.905 | 15.31 | 17.90 | 0.75 g | 0.905 g | 1.5 | 1.81 | g | 3 | 3.62 | g |
| Glutamine | 2 | 2 | 40.82 | 39.56 | 2.00 g | 2.00 g | 4 | 4 | g | 8 | 8 | g |
| NAC | 0.15 | 0.15 | 3.06 | 2.97 | 0.15 g | 0.15 g | 0.3 | 0.3 | g | 0.6 | 0.6 | g |
| Total AAs | | | | | 4.9 g | 5.06 g | 9.8 g | ~10 | g | 19.6 g | ~20 | g |

In some embodiments, the composition (e.g., the Active Moiety) includes 1 g+/−20% of a leucine amino acid entity, 0.5 g+/−20% of an isoleucine amino acid entity, 0.5+/−20% g of a valine amino acid entity, 0.75 g+/−20% of an arginine amino acid entity, 2 g+/−20% of a glutamine amino acid entity, and 0.15 g+/−20% of a NAC-entity. In some embodiments, the composition includes 1 g+/−15% of a leucine amino acid entity, 0.5 g+/−15% of an isoleucine amino acid entity, 0.5+/−15% g of a valine amino acid entity, 0.75 g+/−15% of an arginine amino acid entity, 2 g+/−15% of a glutamine amino acid entity, and 0.15 g+/−15% of a NAC-entity. In some embodiments, the composition includes 1 g+/−10% of a leucine amino acid entity, 0.5 g+/−10% of an isoleucine amino acid entity, 0.5+/−10% g of a valine amino acid entity, 0.75 g+/−10% of an arginine amino acid entity, 2 g+/−10% of a glutamine amino acid entity, and 0.15 g+/−10% of a NAC-entity. In some embodiments, the composition includes 1 g+/−5% of a leucine amino acid entity, 0.5 g+/−5% of an isoleucine amino acid entity, 0.5+/−5% g of a valine amino acid entity, 0.75 g+/−5% of an arginine amino acid entity, 2 g+/−5% of a glutamine amino acid entity, and 0.15 g+/−5% of a NAC-entity. In some embodiments, the composition includes 1 g of a leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.15 g of a NAC-entity.

In some embodiments, the composition (e.g., the Active Moiety) includes 1 g+/−20% of a leucine amino acid entity, 0.5 g+/−20% of an isoleucine amino acid entity, 0.5+/−20% g of a valine amino acid entity, 0.75 g+/−20% of an arginine amino acid entity, 2 g+/−20% of a glutamine amino acid entity, and 0.3 g+/−20% of a NAC-entity. In some embodiments, the composition includes 1 g+/−15% of a leucine amino acid entity, 0.5 g+/−15% of an isoleucine amino acid entity, 0.5+/−15% g of a valine amino acid entity, 0.75 g+/−15% of an arginine amino acid entity, 2 g+/−15% of a glutamine amino acid entity, and 0.3 g+/−15% of a NAC-entity. In some embodiments, the composition includes 1 g+/−10% of a leucine amino acid entity, 0.5 g+/−10% of an isoleucine amino acid entity, 0.5+/−10% g of a valine amino acid entity, 0.75 g+/−10% of an arginine amino acid entity, 2 g+/−10% of a glutamine amino acid entity, and 0.3 g+/−10% of a NAC-entity. In some embodiments, the composition includes 1 g+/−5% of a leucine amino acid entity, 0.5 g+/−5% of an isoleucine amino acid entity, 0.5+/−5% g of a valine amino acid entity, 0.75 g+/−5% of an arginine amino acid entity, 2 g+/−5% of a glutamine amino acid entity, and 0.3 g+/−5% of a NAC-entity. In some embodiments, the composition includes 1 g of a leucine amino acid entity, 0.5 g of an isoleucine amino acid entity, 0.5 g of a valine amino acid entity, 0.75 g or 0.905 g of an arginine amino acid entity, 2 g of a glutamine amino acid entity, and 0.3 g of a NAC-entity.

An exemplary composition can include 1 g of a leucine amino acid entity, 0.5 g of an isoleucine amino acid entity, 0.25 g of a valine amino acid entity, 0.75 g or 0.905 g of an arginine amino acid entity, 1 g of a glutamine amino acid entity, and 0.225 g of a NAC-entity (e.g., g/packet as shown in Table 5).

TABLE 5
Exemplary composition including the form L-arginine (R) or L-arginine HCl (R HCl)).

| | wt. ratio | wt. ratio | wt. % | wt. % | g/packet | | g/packet | | g dose #1 | g dose #1 | | g dose#2 | g dose#2 | |
|---|---|---|---|---|---|---|---|---|---|---|---|---|---|---|
| Amino acid | (R) | (R HCl) | (R) | (R HCl) | (R) | | (R HCl) | | (R) | (R HCl) | | (R) | (R HCl) | |
| Leucine | 1 | 1 | 26.85 | 25.77 | 1.00 | g | 1.00 | g | 2 | 2 | g | 4 | 4 | g |
| Isoleucine | 0.5 | 0.5 | 13.42 | 12.89 | 0.50 | g | 0.50 | g | 1 | 1 | g | 2 | 2 | g |
| Valine | 0.25 | 0.25 | 6.71 | 6.44 | 0.25 | g | 0.25 | g | 0.5 | 0.50 | g | 1 | 1 | g |
| Arginine | 0.75 | 0.905 | 20.13 | 23.32 | 0.75 | g | 0.905 | g | 1.5 | 1.81 | g | 3 | 3.62 | g |
| Glutamine | 1 | 1 | 26.85 | 25.77 | 1.00 | g | 1.00 | g | 2 | 2 | g | 4 | 4 | g |
| NAC | 0.225 | 0.225 | 6.04 | 5.80 | 0.225 | g | 0.225 | g | 0.45 | 0.45 | g | 0.9 | 0.9 | g |
| Total AAs | | | | | 3.725 | g | 3.88 | g | 7.45 g | 7.76 | g | 14.9 g | 15.52 | g |

In some embodiments, the composition (e.g., the Active Moiety) includes 1 g+/−20% of a leucine amino acid entity, 0.5 g+/−20% of an isoleucine amino acid entity, 0.25+/−20% g of a valine amino acid entity, 0.75 g+/−20% of an arginine amino acid entity, 1 g+/−20% of a glutamine amino acid entity, and 0.225 g+/−20% of a NAC-entity. In some embodiments, the composition includes 1 g+/−15% of a leucine amino acid entity, 0.5 g+/−20% of an isoleucine amino acid entity, 0.25+/−20% g of a valine amino acid entity, 0.75 g+/−15% of an arginine amino acid entity, 1 g+/−15% of a glutamine amino acid entity, and 0.225 g+/−15% of a NAC-entity. In some embodiments, the composition includes 1 g+/−10% of a leucine amino acid entity, 0.5 g+/−20% of an isoleucine amino acid entity, 0.25+/−20% g of a valine amino acid entity, 0.75 g+/−10% of an arginine amino acid entity, 1 g+/−10% of a glutamine amino acid entity, and 0.225 g+/−10% of a NAC-entity. In some embodiments, the composition includes 1 g+/−5% of a leucine amino acid entity, 0.5 g+/−20% of an isoleucine amino acid entity, 0.25+/−20% g of a valine amino acid entity, 0.75 g+/−5% of an arginine amino acid entity, 1 g+/−5% of a glutamine amino acid entity, and 0.225 g+/−5% of a NAC-entity. An exemplary composition can include 1 g of a leucine amino acid entity, 0.5 g of an isoleucine amino acid entity, 0.25 g of a valine amino acid entity, 0.75 g or 0.905 g of an arginine amino acid entity, 1 g of a glutamine amino acid entity, 0.225 g of a NAC-entity, and 1.5 g of the serine amino acid entity (e.g., g/packet as shown in Table 6).

TABLE 6
Exemplary composition including the form L-arginine (R) or L-arginine HCl (R HCl)).

| | wt. ratio | wt. ratio | wt. % | wt. % | g/packet | g/packet | | g dose #1 | g dose #1 | | g dose#2 | g dose#2 | |
|---|---|---|---|---|---|---|---|---|---|---|---|---|---|
| Amino acid | (R) | (R HCl) | (R) | (R HCl) | (R) | (R HCl) | | (R) | (R HCl) | | (R) | (R HCl) | |
| Leucine | 1 | 1 | 19.14 | 18.59 | 1.00 g | 1.00 | g | 2 | 2 | g | 4 | 4 | g |
| Isoleucine | 0.5 | 0.5 | 9.57 | 9.29 | 0.50 g | 0.50 | g | 1 | 1 | g | 2 | 2 | g |
| Valine | 0.25 | 0.25 | 4.78 | 4.65 | 0.25 g | 0.25 | g | 0.5 | 0.50 | g | 1 | 1 | g |
| Arginine | 0.75 | 0.905 | 14.35 | 16.82 | 0.75 g | 0.905 | g | 1.5 | 1.81 | g | 3 | 3.62 | g |
| Glutamine | 1 | 1 | 19.14 | 18.59 | 1.00 g | 1.00 | g | 2 | 2 | g | 4 | 4 | g |
| NAC | 0.225 | 0.225 | 4.31 | 4.18 | 0.225 g | 0.225 | g | 0.45 | 0.45 | g | 0.9 | 0.9 | g |
| Serine | 1.5 | 1.5 | 28.71 | 27.88 | 1.5 | 1.5 | | 3 | 3 | | 6 | 6 | |
| Total AAs | | | | | 5.225 | 5.38 | g | 10.45 | 10.76 | g | 20.9 | 21.52 | g |

In some embodiments, the composition comprises 1 g+/−20% of the leucine amino acid entity, 0.5 g+/−20% of the isoleucine amino acid entity, 0.25 g+/−20% of the valine amino acid entity, 0.75 g+/−20% of the arginine amino acid entity, 1 g+/−20% of the glutamine amino acid entity, 0.225 g+/−20% of the NAC-amino acid entity, and 1.5 g+/−20% of the serine amino acid entity. In some embodiments, the composition comprises 1 g+/−15% of the leucine amino acid entity, 0.5 g+/−15% of the isoleucine amino acid entity, 0.25 g+/−15% of the valine amino acid entity, 0.75 g+/−15% of the arginine amino acid entity, 1 g+/−15% of the glutamine amino acid entity, 0.225 g+/−15% of the NAC-amino acid entity, and 1.5 g+/−15% of the serine amino acid entity. In some embodiments, the composition comprises 1 g+/−10% of the leucine amino acid entity, 0.5 g+/−10% of the isoleucine amino acid entity, 0.25 g+/−10% of the valine amino acid entity, 0.75 g+/−10% of the arginine amino acid entity, 1 g+/−10% of the glutamine amino acid entity, 0.225 g+/−10% of the NAC-amino acid entity, and 1.5 g+/−10% of the serine amino acid entity. In some embodiments, the composition comprises 1 g+/−5% of the leucine amino acid entity, 0.5 g+/−5% of the isoleucine amino acid entity, 0.25 g+/−5% of the valine amino acid entity, 0.75 g+/−5% of the arginine amino acid entity, 1 g+/−5% of the glutamine amino acid entity, 0.225 g+/−5% of the NAC-amino acid entity, and 1.5 g+/−5% of the serine amino acid entity.

An exemplary composition can include 1 g of a leucine amino acid entity, 0.5 g of an isoleucine amino acid entity, 0.25 g of a valine amino acid entity, 0.75 g or 0.905 g of an arginine amino acid entity, 1 g of a glutamine amino acid entity, 0.225 g of a NAC-entity, and 1.667 g of the serine amino acid entity (e.g., g/packet as shown in Table 7).

TABLE 7
Exemplary composition including the form L-arginine (R) or L-arginine HCl (R HCl)).

| | wt. ratio | wt. ratio | wt. % | wt. % | g/packet | | g/packet | | g dose #1 | g dose #1 | | g dose#2 | g dose#2 | |
|---|---|---|---|---|---|---|---|---|---|---|---|---|---|---|
| Amino acid | (R) | (R HCl) | (R) | (R HCl) | (R) | | (R HCl) | | (R) | (R HCl) | | (R) | (R HCl) | |
| Leucine | 1 | 1 | 18.54 | 18.02 | 1.00 | g | 1.00 | g | 2 | 2 | g | 4 | 4 | g |
| Isoleucine | 0.5 | 0.5 | 9.27 | 9.01 | 0.50 | g | 0.50 | g | 1 | 1 | g | 2 | 2 | g |
| V aline | 0.25 | 0.25 | 4.64 | 4.50 | 0.25 | g | 0.25 | g | 0.5 | 0.50 | g | 1 | 1 | g |
| Arginine | 0.75 | 0.905 | 13.91 | 16.31 | 0.75 | g | 0.905 | g | 1.5 | 1.81 | g | 3 | 3.62 | g |
| Glutamine | 1 | 1 | 18.54 | 18.02 | 1.00 | g | 1.00 | g | 2 | 2 | g | 4 | 4 | g |
| NAC | 0.225 | 0.225 | 4.17 | 4.05 | 0.225 | g | 0.225 | g | 0.45 | 0.45 | g | 0.9 | 0.9 | g |
| Serine | 1.667 | 1.667 | 30.92 | 30.09 | 1.67 | g | 1.67 | g | 3.33 g | 3.33 | g | 6.67 g | 6.67 | g |
| Total AAs | | | | | 5.395 | g | 5.55 | g | 10.78 g | 11.09 | g | 21.57 g | 22.19 | g |

In some embodiments, the composition comprises 1 g+/−20% of the leucine amino acid entity, 0.5 g+/−20% of the isoleucine amino acid entity, 0.25 g+/−20% of the valine amino acid entity, 0.75 g+/−20% of the arginine amino acid entity, 1 g+/−20% of the glutamine amino acid entity, 0.225 g+/−20% of the NAC-amino acid entity, and 1.667 g+/−20% of the serine amino acid entity. In some embodiments, the composition comprises 1 g+/−15% of the leucine amino acid entity, 0.5 g+/−15% of the isoleucine amino acid entity, 0.25 g+/−15% of the valine amino acid entity, 0.75 g+/−15% of the arginine amino acid entity, 1 g+/−15% of the glutamine amino acid entity, 0.225 g+/−15% of the NAC-amino acid entity, and 1.667 g+/−15% of the serine amino acid entity. In some embodiments, the composition comprises 1 g+/−10% of the leucine amino acid entity, 0.5 g+/−10% of the isoleucine amino acid entity, 0.25 g+/−10% of the valine amino acid entity, 0.75 g+/−10% of the arginine amino acid entity, 1 g+/−10% of the glutamine amino acid entity, 0.225 g+/−10% of the NAC-amino acid entity, and 1.667 g+/−10% of the serine amino acid entity. In some embodiments, the composition comprises 1 g+/−5% of the leucine amino acid entity, 0.5 g+/−5% of the isoleucine amino acid entity, 0.25 g+/−5% of the valine amino acid entity, 0.75 g+/−5% of the arginine amino acid entity, 1 g+/−5% of the glutamine amino acid entity, 0.225 g+/−5% of the NAC-amino acid entity, and 1.667 g+/−5% of the serine amino acid entity.

An exemplary composition can include 1 g of a leucine amino acid entity, 0.5 g of an isoleucine amino acid entity, 0.5 g of a valine amino acid entity, 1.5 g or 1.81 g of an arginine amino acid entity, 1.33 g of a glutamine amino acid entity, 0.15 g of a NAC-entity, 0.08 g of a histidine amino acid entity, 0.35 g of a lysine amino acid entity, 0.08 g of a phenylalanine amino acid entity, and 0.17 g of a threonine amino acid entity (e.g., g/packet as shown in Table 8).

TABLE 8
Exemplary composition including the form L-
arginine (R) or L-arginine HCl (R HCl)).

| | | | | Total g | | Total g |
|---|---|---|---|---|---|---|
| | weight | | | daily | | daily |
| Amino acid | ratio | g/packet | g/dose 1 | dose 1 | g/dose 2 | dose 2 |
| Leucine | 2.0 | 1.0 | 1.0 | 3 | 4 | 12 |
| Isoleucine | 1.0 | 0.5 | 0.5 | 1.5 | 2 | 6 |
| Valine | 1.0 | 0.5 | 0.5 | 1.5 | 2 | 6 |
| Arginine HCl | 3.62 | 1.81 | 1.81 | 5.43 | 7.24 | 21.72 |
| Glutamine | 2.66 | 1.33 | 1.33 | 3.99 | 5.32 | 15.96 |
| N- | 0.3 | 0.15 | 0.15 | 0.45 | 0.6 | 1.8 |
| acetylcysteine | | | | | | |
| Histidine | 0.16 | 0.08 | 0.08 | 0.24 | 0.32 | 0.96 |
| Lysine | 0.7 | 0.35 | 0.35 | 1.05 | 1.4 | 4.2 |
| Phenylalanine | 0.16 | 0.08 | 0.08 | 0.24 | 0.32 | 0.96 |
| Threonine | 0.34 | 0.17 | 0.17 | 0.51 | 0.68 | 2.04 |
| Total amino | | ~6 g | ~6 g | ~18 g | ~24 g | ~72 g |
| acids | | | | | | |

In some embodiments, the composition comprises 1 g+/−20% of a leucine amino acid entity, 0.5 g+/−20% of an isoleucine amino acid entity, 0.5 g+/−20% of a valine amino acid entity, 1.5 g or 1.81 g+/−20% of an arginine amino acid entity, 1.33 g+/−20% of a glutamine amino acid entity, 0.15 g+/−20% of a NAC-entity, 0.08 g+/−20% of a histidine amino acid entity, 0.35 g+/−20% of a lysine amino acid entity, 0.08 g+/−20% of a phenylalanine amino acid entity, and 0.17 g+/−20% of a threonine amino acid entity. In some embodiments, the composition comprises 1 g+/−15% of a leucine amino acid entity, 0.5 g+/−15% of an isoleucine amino acid entity, 0.5 g+/−15% of a valine amino acid entity, 1.5 g or 1.81 g+/−15% of an arginine amino acid entity, 1.33 g+/−15% of a glutamine amino acid entity, 0.15 g+/−15% of a NAC-entity, 0.08 g+/−15% of a histidine amino acid entity, 0.35 g+/−15% of a lysine amino acid entity, 0.08 g+/−15% of a phenylalanine amino acid entity, and 0.17 g+/−15% of a threonine amino acid entity.

ii. Ratios

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.5+/−15%:1.5+/−15%:2+/−15%:0.15+/−15% or 1+/−15%:0.5+/−15%:0.5+/−15%: 1.81+/−15%:2+/−15%:0.15+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.5+/−10%:1.5+/−10%:2+/−10%:0.15+/−10% or 1+/−10%:0.5+/−10%:0.5+/−10%:1.81+/−10%:2+/−10%:0.15+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.5+/−5%:1.5+/−5%:2+/−5%: 0.15+/−5% or 1+/−5%:0.5+/−5%:0.5+/−5%:1.81+/−5%:2+/−5%:0.15+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:1.5:2:0.15 or 1:0.5:0.5:1.81:2:0.15.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%:0.5+/−20%:0.5+/−20%:1.5+/−20%:2+/−20%:0.3+/−20% or 1+/−20%:0.5+/−20%:0.5+/−20%: 1.81+/−20%:2+/−20%:0.3+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.5+/−15%:1.5+/−15%:2+/−15%:0.3+/−15% or 1+/−15%: 0.5+/−15%:0.5+/−15%:1.81+/−15%:2+/−15%:0.3+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.5+/−10%:1.5+/−10%:2+/−10%:0.3+/−10% or 1+/−10%:0.5+/−10%:0.5+/−10%:1.81+/−10%:2+/−10%:0.3+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.5+/−5%: 1.5+/−5%:2+/−5%:0.3+/−5% or 1+/−5%:0.5+/−5%:0.5+/−5%:1.81+/−5%:2+/−5%: 0.3+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:1.5:2:0.3 or 1:0.5:0.5:1.81:2:0.3.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%:0.5+/−20%:0.5+/−20%:0.75+/−20%:2+/−20%:0.15+/−20% or 1+/−20%:0.5+/−20%:0.5+/−20%: 0.905+/−20%:2+/−20%:0.15+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.5+/−15%:0.75+/−15%:2+/−15%:0.15+/−15% or 1+/−15%:0.5+/−15%:0.5+/−15%:0.905+/−15%:2+/−15%:0.15+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.5+/−10%:0.75+/−10%:2+/−10%:0.15+/−10% or 1+/−10%:0.5+/−10%:0.5+/−10%:0.905+/−10%:2+/−10%:0.15+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.5+/−5%:0.75+/−5%:2+/−5%:0.15+/−5% or 1+/−5%:0.5+/−5%: 0.5+/−5%:0.905+/−5%:2+/−5%:0.15+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:0.75:2:0.15 or 1:0.5:0.5:0.905:2:0.15.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%:0.5+/−20%:0.5+/−20%:0.75+/−20%:2+/−20%:0.3+/−20% or 1+/−20%:0.5+/−20%:0.5+/−20%: 0.905+/−20%:2+/−20%:0.3+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.5+/−15%:0.75+/−15%:2+/−15%:0.3+/−15% or 1+/−15%:0.5+/−15%:0.5+/−15%:0.905+/−15%:2+/−15%:0.3+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.5+/−10%:0.75+/−10%: 2+/−10%:0.3+/−10% or 1+/−10%:0.5+/−10%:0.5+/−10%:0.905+/−10%:2+/−10%:0.3+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%: 0.5+/−5%:0.75+/−5%:2+/−5%:0.3+/−5% or 1+/−5%:0.5+/−5%:0.5+/−5%:0.905+/−5%: 2+/−5%:0.3+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.5:0.75:2:0.3 or 1:0.5:0.5:0.905:2:0.3.

An exemplary composition can include a weight (wt.) ratio of the leucine amino acid entity, the isoleucine amino acid entity, the valine amino acid entity, the arginine amino acid entity, the glutamine amino acid entity, and the NAC-amino acid entity of 1+/−20%:0.5+/−20%:0.25+/−20%:0.75+/−20%:1+/−20%:0.225+/−20% or 1+/−20%:0.5+/−20%:0.25+/−20%:0.905+/−20%:1+/−20%:0.225+/−20%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−15%:0.5+/−15%:0.25+/−15%:0.75+/−15%:1+/−15%:0.225+/−15% or 1+/−15%:0.5+/−15%:0.25+/−15%:0.905+/−15%:1+/−15%:0.225+/−15%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−10%:0.5+/−10%:0.25+/−10%:0.75+/−10%:1+/−10%:0.225+/−10% or 1+/−10%:0.5+/−10%:0.25+/−10%:0.905+/−10%:1+/−10%:0.225+/−10%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1+/−5%:0.5+/−5%:0.25+/−5%:0.75+/−5%:1+/−5%:0.225+/−5% or 1+/−5%:0.5+/−5%: 0.25+/−5%:0.905+/−5%:1+/−5%:0.225+/−5%. In some embodiments, the composition includes a wt. ratio of the leucine amino acid entity, the isoleucine amino acid entity, the valine amino acid entity, the arginine amino acid entity, the glutamine amino acid entity, and the NAC-amino acid entity of 1:0.5:0.25:0.75:1:0.225 or 1:0.5:0.25:0.905:1:0.225.

An exemplary composition comprising amino acid entities can include a weight (wt.) ratio of the leucine amino acid entity, the isoleucine amino acid entity, the valine amino acid entity, the arginine amino acid entity, the glutamine amino acid entity, the NAC-amino acid entity, and the serine amino acid entity of 1+/−20%:0.5+/−20%:0.25+/−20%:0.75+/−20%: 1+/−20%:0.225+/−20%:1.5+/−20% or 1+/−20%:0.5+/−20%:0.25+/−20%:0.905+/−20%: 1+/−20%:0.225+/−20%:1.5+/−20%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−15%:0.5+/−15%:0.25+/−15%:0.75+/−15%: 1+/−15%:0.225+/−15%:1.5+/−15% or 1+/−15%:0.5+/−15%:0.25+/−15%:0.905+/−15%: 1+/−15%:0.225+/−15%:1.5+/−15%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−10%:0.5+/−10%:0.25+/−10%:0.75+/−10%: 1+/−10%:0.225+/−10%:1.5+/−10% or 1+/−10%:0.5+/−10%:0.25+/−10%:0.905+/−10%: 1+/−10%:0.225+/−10%:1.5+/−10%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−5%:0.5+/−5%:0.25+/−5%:0.75+/−5%:1+/−5%:0.225+/−5%:1.5+/−5% or 1+/−5%:0.5+/−5%:0.25+/−5%:0.905+/−5%:1+/−5%: 0.225+/−5%:1.5+/−5%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1:0.5:0.25:0.75:1:0.225:1.5 or 1:0.5:0.25:0.905:1:0.225:1.5.

An exemplary composition can include a weight (wt.) ratio of the leucine amino acid entity, the isoleucine amino acid entity, the valine amino acid entity, the arginine amino acid entity, the glutamine amino acid entity, the NAC-amino acid entity, and the serine amino acid entity of 1+/−20%:0.5+/−20%:0.25+/−20%:0.75+/−20%:1+/−20%:0.225+/−20%: 1.667+/−20% or 1+/−20%:0.5+/−20%:0.25+/−20%:0.905+/−20%:1+/−20%:0.225+/−20%:1.667+/−20%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−15%:0.5+/−15%:0.25+/−15%:0.75+/−15%:1+/−15%:0.225+/−15%:1.667+/−15% or 1+/−15%:0.5+/−15%:0.25+/−15%:0.905+/−15%:1+/−15%: 0.225+/−15%:1.667+/−15%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−10%:0.5+/−10%:0.25+/−10%:0.75+/−10%:1+/−10%: 0.225+/−10%:1.667+/−10% or 1+/−10%:0.5+/−10%:0.25+/−10%:0.905+/−10%:1+/−10%:0.225+/−10%:1.667+/−10%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1+/−5%:0.5+/−5%:0.25+/−5%:0.75+/−5%:1+/−5%: 0.225+/−5%:1.667+/−5% or 1+/−5%:0.5+/−5%:0.25+/−5%:0.905+/−5%:1+/−5%: 0.225+/−5%:1.667+/−5%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, and the serine amino acid entity of 1:0.5:0.25:0.75:1:0.225:1.667 or 1:0.5:0.25:0.905:1:0.225:1.667.

An exemplary composition can include a weight (wt.) ratio of the leucine amino acid entity, the isoleucine amino acid entity, the valine amino acid entity, the arginine amino acid entity, the glutamine amino acid entity, the NAC-amino acid entity, the histidine amino acid entity, the lysine amino acid entity, the phenylalanine amino acid entity, and the threonine amino acid entity of 2+/−20%:1+/−20%:1+/−20%:3.62+/−20%:2.66+/−20%:0.3+/−20%: 0.16+/−20%:0.7+/−20%:0.16+/−20%:0.34+/−20% or 2+/−20%:1+/−20%:1+/−20%:3+/−20%:2.66+/−20%:0.3+/−20%:0.16+/−20%:0.7+/−20%:0.16+/−20%:0.34+/−20%. In some embodiments, the composition includes a wt. ratio of the leucine amino acid entity, the isoleucine amino acid entity, the valine amino acid entity, the arginine amino acid entity, the glutamine amino acid entity, the NAC-amino acid entity, the histidine amino acid entity, the lysine amino acid entity, the phenylalanine amino acid entity, and the threonine amino acid entity of 2+/−15%:1+/−15%:1+/−15%:3.62+/−15%:2.66+/−15%:0.3+/−15%:0.16+/−15%: 0.7+/−15%:0.16+/−15%:0.34+/−15% or 2+/−15%:1+/−15%:1+/−15%:3+/−15%:2.66+/−15%:0.3+/−15%:0.16+/−15%:0.7+/−15%:0.16+/−15%:0.34+/−15%.

In some embodiments, the composition includes 10 wt. %+/−15% to 30 wt. %+/−15% of a leucine amino acid entity, 5 wt. %+/−15% to 15 wt. %+/−15% of a isoleucine amino acid entity, 5 wt. %+/−15% to 15 wt. %+/−15% of a valine amino acid entity, 15 wt. %+/−15% to 40 wt. %+/−15% of an arginine amino acid entity, 20 wt. %+/−15% to 50 wt. %+/−15% of a glutamine amino acid entity, and 1 wt. %+/−15% to 8 wt. %+/−15% of a NAC entity.

In some embodiments, the composition includes 10 wt. %+/−15% to 30 wt. %+/−15% of a leucine amino acid entity. In some embodiments, the composition includes 5 wt. %+/−15% to 15 wt. %+/−15% of a isoleucine amino acid entity. In some embodiments, the composition includes 5 wt. %+/−15% to 15 wt. %+/−15% of a valine amino acid entity. In some embodiments, the composition includes 15 wt. %+/−15% to 40 wt. %+/−15% of an arginine amino acid entity. In some embodiments, the composition includes 20 wt. %+/−15% to 50 wt. %+/−15% of a glutamine amino acid entity. In some embodiments, the composition includes 1 wt. %+/−15% to 8 wt. %+/−15% of a NAC entity.

In some embodiments, the composition includes 16 wt. %+/−15% to 18 wt. %+/−15% of a leucine amino acid entity, 7 wt. %+/−15% to 9 wt. %+/−15% of a isoleucine amino acid entity, 7 wt. %+/−15% to 9 wt. %+/−15% of a valine amino acid entity, 28 wt. %+/−15% to 32 wt. %+/−15% of an arginine amino acid entity, 31 wt. %+/−15% to 34 wt. %+/−15% of a glutamine amino acid entity, and 1 wt. %+/−15% to 5 wt. %+/−15% of a NAC-entity.

In some embodiments, the composition includes 16 wt. %+/−15% to 18 wt. %+/−15% of a leucine amino acid entity. In some embodiments, the composition includes 7 wt. %+/−15% to 9 wt. %+/−15% of a isoleucine amino acid entity. In some embodiments, the composition includes 7 wt. %+/−15% to 9 wt. %+/−15% of a valine amino acid entity. In some embodiments, the composition includes 28 wt. %+/−15% to 32 wt. %+/−15% of an arginine amino acid entity. In some embodiments, the composition includes 31 wt. %+/−15% to 34 wt. %+/−15% of a glutamine amino acid entity. In some embodiments, the composition includes 1 wt. %+/−15% to 5 wt. %+/−15% of a NAC-entity.

In some embodiments, the composition includes 16.8 wt. %+/−15% of a leucine amino acid entity, 8.4 wt. %+/−15% of a isoleucine amino acid entity, 8.4 wt. %+/−15% of a valine amino acid entity, 30.4 wt. %+/−15% of an arginine amino acid entity, 33.6 wt. %+/−15% of a glutamine amino acid entity, and 2.5 wt. %+/−15% of a NAC-entity.

iii. Relationships of Amino Acid Entities

In some embodiments, the composition (e.g., the Active Moiety) has one or more of the following properties:

a) a wt. % of the Q-amino acid entity in the composition is greater than the wt. % of the R-amino acid entity;
b) the wt. % of the Q-amino acid entity in the composition is greater than the wt. % of the L-amino acid entity;
c) the wt. % of the R-amino acid entity in the composition is greater than the wt. % of the L-amino acid entity; or
d) a combination of two or three of (a)-(c).

In some embodiments, the wt. % of the glutamine amino acid entity in the composition is greater than the wt. % of the arginine amino acid entity, e.g., the wt. % of the glutamine amino acid entity in the composition is at least 5% greater than the wt. % of the arginine amino acid entity, e.g., the wt. % of the glutamine amino acid entity is at least 10% or 25% greater than the wt. % of the arginine amino acid entity.

In some embodiments, the wt. % of the glutamine amino acid entity in the composition is greater than the wt. % of the leucine amino acid entity, e.g., the wt. % of the glutamine amino acid entity in the composition is at least 20% greater than the wt. % of the leucine amino acid entity, e.g., the wt. % of the glutamine amino acid entity in the composition is at least 25% 50% greater than the wt. % of the leucine amino acid entity.

In some embodiments, the wt. % of the arginine amino acid entity in the composition is greater than the wt. % of the leucine amino acid entity, e.g., the wt. % of the arginine amino acid entity in the composition is at least 10% greater than the wt. % of the leucine amino acid entity, e.g., the wt. % of the arginine amino acid entity in the composition is at least 15% or 30% greater than the wt. % of the leucine amino acid entity.

In some embodiments, the wt. % of the leucine amino acid entity in the composition is greater than the wt. % of the isoleucine amino acid entity in the composition, e.g., the wt. % of the leucine amino acid entity in the composition is at least 25 wt. % greater than the wt. % of the isoleucine amino acid entity in the composition.

In some embodiments, the wt. % of the leucine amino acid entity in the composition is greater than the wt. % of the valine amino acid entity in the composition, e.g., the wt. % of the leucine amino acid entity in the composition is at least 25 wt. % greater than the wt. % of the valine amino acid entity in the composition.

In some embodiments, the wt. % of the arginine amino acid entity, the glutamine amino acid entity, and the NAC entity is at least:50 wt. % or 70 wt. % of the amino acid entities in the composition, but not more than 90 wt. % of the amino acid entities in the composition.

In some embodiments, the wt. % of the NAC entity is at least:1 wt. % or 2 wt. % of the amino acid entity components or total components in the composition, but not more than 10 wt. % or more of the amino acid entity components or total components in the composition.

In some embodiments, the isoleucine amino acid entity, and the valine amino acid entity in combination is at least:15 wt. %, or 20 wt. % of the amino acid entity components or total components in the composition, but not more than:50 wt. % of the amino acid entity components or total components in the composition;

In some embodiments, the glutamine amino acid entity, and the NAC entity is at least:40 wt. % or 50 wt. % of the amino acid entity components or total components in the composition, but not more than 90 wt. % of the amino acid entity components or total components in the composition.

In some embodiments, the composition (e.g., the Active Moiety) further comprises an serine amino acid entity, e.g., the serine amino acid entity is present at a higher amount than any other amino acid entity component in the composition. In some embodiments, the wt. % of the serine amino acid entity is at least 20 wt. % or more of the amino acid entities or total components in the composition.

iv. Amino Acid Molecules to Exclude or Limit from the Composition

In some embodiments, the composition does not comprise a peptide of more than 20 amino acid residues in length (e.g., protein supplement) chosen from or derived from one, two, three, four, five, or more (e.g., all) of egg white protein, soy protein, casein, hemp protein, pea protein, or brown rice protein, or if the peptide is present, the peptide is present at less than:10 weight (wt.) 5 wt. %, 1 wt. %, 0.1 wt. %, 0.05 wt. %, 0.01 wt. %, of the total wt. of non-amino acid entity protein components or total components in the composition (e.g., in dry form).

In some embodiments, the composition comprises a combination of 3 to 19, 3 to 15, or 3 to 10 different amino acid entities; e.g., the combination comprises at least:42 wt. %, 75 wt. %, or 90 wt. % of the total wt. % of amino acid entity components or total components in the composition (e.g., in dry form).

In some embodiments, dipeptides or salts thereof or tripeptides or salts thereof are present at less than:10 wt. %, 0.5 wt. %, 0.1 wt. %, 0.05 wt. %, 0.01 wt. %, 0.001 wt. %, or less of the total wt. of amino acid entity components or total components in the composition (e.g., in dry form).

In some embodiments, at least 50%, 60%, 70%, or more of the total grams of amino acid entity components in the composition (e.g., in dry form) are from one, two, three, four, five, seven, eight, nine, or more (e.g., all) of (a)-(j).

In some embodiments, at least:50%, 60%, 70%, or more of the calories from amino acid entity components or total components in the composition (e.g., in dry form) are from one, two, three, four, five, seven, eight, nine, or more (e.g., all) of (a)-(j).

In some embodiments, a carbohydrate (e.g., one, two, three, four, five, six, seven, eight, nine, 10, 11, 12, 13, 14, 15, 16, 17, or 18 of dextrose, maltodextrose, sucrose, dextrin, fructose, galactose, glucose, glycogen, high fructose corn syrup, honey, inositol, invert sugar, lactose, levulose, maltose, molasses, sugarcane, or xylose) is absent from the composition, or if present, is present at less than:10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a vitamin (e.g., one, two, three, four, five, six, or seven of vitamin B1, vitamin B2, vitamin B3, vitamin B6, vitamin B12, vitamin C, or vitamin D) is absent from the composition, or if present, is present at less than:10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one or both of nitrate or nitrite are absent from the composition, or if present, are present at less than:10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, 4-hydroxyisoleucine is absent from the composition, or if present, is present at less than:10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, a probiotic (e.g., a Bacillus probiotic) is absent from the composition, or if present, is present at less than:10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, phenylacetate is absent from the composition, or if present, is present at less than:10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, gelatin (e.g., a gelatin capsule) is absent from the composition, or if present, is present at less than:10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

In some embodiments, one, two, or three of S-allyl cysteine, S-allylmercaptocysteine, or fructosyl-arginine is absent from the composition, or if present, is present at less than:10 wt. %, 5 wt. %, 1 wt. %, 0.5 wt. %, 0.1 wt. %, 0.05 wt. %, 0.01 wt. %, 0.001 wt. %, or less, e.g., of the total wt. of the composition (in dry form).

### Uses, e.g., Methods of Treatment

The composition of the invention as described herein (e.g., the Active Moiety) can be administered to improve or reduce insulin resistance, e.g., treat or prevent insulin resistance in a subject. The composition can be administered to improve impaired glucose tolerance, e.g., in a patient with insulin resistance. The composition can be administered according to a dosage regimen described herein to treat a subject with one or both of insulin resistance or impaired glucose tolerance.

The disclosure features a method for improving or reducing insulin resistance or impaired glucose tolerance, comprising administering to a subject in need thereof an effective amount of a composition disclosed herein (e.g., an Active Moiety). The method includes administering the composition described herein to a subject in need thereof, in an amount sufficient to decrease or inhibit insulin resistance or impaired glucose tolerance in the subject.

In some embodiments, the composition described herein (e.g., the Active Moiety) is for use as a medicament in treating (e.g., reversing, reducing, ameliorating, or preventing) one or both of insulin resistance or impaired glucose tolerance in a subject. In some embodiments, the composition described herein (e.g., the Active Moiety) is for use in the manufacture of a medicament for treating (e.g., reversing, reducing, ameliorating, or preventing) one or both of insulin resistance or impaired glucose tolerance in a subject.

In some embodiments, the subject has insulin resistance or has been diagnosed with insulin resistance. In some embodiments the subject has impaired glucose tolerance or has been diagnosed with impaired glucose tolerance (e.g., hyperglycemia).

In some embodiments, the subject is a human. In some embodiments, the subject has not received prior treatment with the composition (e.g., a naïve subject).

In certain embodiments, administration of the composition results in one, two, three, or more (e.g., all) of: increased free fatty acid metabolism, increased lipid metabolism, decreased insulin secretion, or increased glucose tolerance. In certain embodiments, administration of the composition results in increased white adipose tissue browning. In certain embodiments, administration of the composition results in decreased steatosis (e.g., macrovesicular steatosis or microvesicular steatosis). In certain embodiments, administration of the composition enhances fatty acid oxidation, e.g., by reducing one or both of unsaturated fatty acid levels or acylcarnitine levels.

In some embodiments, the subject is overweight or obese. In certain embodiments, the subject may have, or may be at risk of having, a disorder in which obesity or being overweight is a risk factor. In some embodiments, the composition promotes weight loss in the subject. In some embodiments, improving one or both of insulin resistance or glucose tolerance increases survival of the subject.

Exemplary conditions and disorders that can be treated with the composition described herein include, but are not limited to, prediabetes; type 2 diabetes; metabolic syndrome; a cardiovascular condition or disorder, such as hypertension, dyslipidemia, atherosclerosis, or obstructive sleep apnea; an endocrine condition or disorder, such as polycystic ovarian syndrome (PCOS), hyperthyroidism, Cushing's disease, Cushing's syndrome, acromegaly, or pheochromocytoma; a genetic condition or disorder, such as Down's syndrome, Turner's syndrome, Klinefelter's syndrome, thalassaemia, haemochromatosis, lipodystrophy, progeria, Huntington's chorea, Friedrich's ataxia, Laurence-Moon-Biedl syndrome, a glycogen storage disease type I, a glycogen storage disease type III, or an inherited mitochondrial disorder; renal failure; pregnancy; a cancer, such as colon cancer, endometrial cancer, pancreatic cancer, renal-cell cancer, or breast cancer; a dementia, such as Alzheimer's disease or Lewy body dementia; myotonic dystrophy; a syndrome of severe insulin resistance (SSIR); or a genetic disorder of insulin resistance, such as Donohue Syndrome, Rabson-Mendenhall Syndrome, or Type A Insulin Resistance.

In some embodiments, the insulin resistance or glucose tolerance is associated with a liver condition or disorder, e.g., a liver condition or disorder chosen from: non-alcoholic fatty liver (NAFL), non-alcoholic fatty liver disease (NAFLD), non-alcoholic steatohepatitis (NASH), alcoholic fatty liver disease (AFLD), alcoholic steatohepatitis (ASH), or cirrhosis.

In some embodiments, the insulin resistance or glucose tolerance is not associated with a liver condition or disorder, e.g., a liver condition or disorder chosen from: non-alcoholic fatty liver (NAFL), non-alcoholic fatty liver disease (NAFLD), non-alcoholic steatohepatitis (NASH), alcoholic fatty liver disease (AFLD), alcoholic steatohepatitis (ASH), or cirrhosis.

In some embodiments, the insulin resistance or glucose tolerance is not associated with a muscle condition or disorder.

### Dosage Regimens

The composition (e.g., the Active Moiety) can be administered according to a dosage regimen described herein to reduce or treat insulin resistance or improve glucose tolerance. For example, the composition may be administered to the subject for a treatment period of, e.g., two weeks, three weeks, four weeks, five weeks, six weeks, seven weeks, eight weeks, nine weeks, 10 weeks, 11 weeks, 12 weeks, 13 weeks, 14 weeks, 15 weeks, 16 weeks, or longer at a dose of 2 g+/−20% g daily to 90 g+/−20% g daily (e.g., 72 g+/−20% total amino acid entities daily).

In some embodiments, the composition can be provided to a subject (e.g., a subject with insulin resistance) in either a single or multiple dosage regimen. In some embodiments, a dose is administered twice daily, three times daily, four times daily, five times daily, six times daily, seven times daily, or more. In certain embodiments, the composition is administered one, two, or three times daily. In some embodiments, the composition is administered for at least 2 days, 3 days, 4 days, 5 days, 6 days, 7 days, or 2 weeks. In some embodiments, the composition is administered chronically (e.g., more than 30 days, e.g., 31 days, 40 days, 50 days, 60 days, 3 months, 6 months, 9 months, one year, two years, or three years).

In some embodiments, the composition is administered prior to a meal. In other embodiments, the composition is administered concurrent with a meal. In other embodiments, the composition is administered following a meal.

The composition can be administered every 2 hours, every 3 hours, every 4 hours, every 5 hours, every 6 hours, every 7 hours, every 8 hours, every 9 hours, or every 10 hours to improve or reduce insulin resistance in a subject (e.g., a subject having a insulin resistance).

In some embodiments, the composition comprises four stick packs, e.g., each stick pack comprising 25%+/−15% of the quantity of each amino acid entity included in the composition described herein. In certain embodiments, four stick packs are administered three times daily. In some embodiments, the composition comprises three stick packs, e.g., each stick pack comprising 33.3%+/−15% of the quantity of each amino acid entity included in the composition described herein. In certain embodiments, three stick packs are administered three times daily.

In some embodiments, the composition is administered at a dose of about 2 g+/−20% to 50 g+/−20% total amino acid entities, e.g., once per day, twice per day, three times per day, four times per day, five times per day, or six times per day (e.g., three times per day). In certain embodiments, the composition is administered at a dose of 2 g+/−20% to 10 g+/−20% total amino acid entities three times daily, e.g., 8 g+/−20% or 10 g+/−20% total amino acid entities three times daily. In certain embodiments, the composition is administered at a dose of 10 g+/−20% to 20 g+/−20% total amino acid entities three times daily, e.g., 11 g+/−20%, 12 g+/−20%, 15 g+/−20%, 16 g+/−20%, or 20 g+/−20% total amino acid entities three times daily. In certain embodiments, the composition is administered at a dose of 20 g+/−20% to 30 g+/−20% total amino acid entities three times daily, e.g., 21 g+/−20%, 22 g+/−20%, 23 g+/−20%, or 24 g+/−20% total amino acid entities three times daily.

### Production of Active Moiety and Pharmaceutical Compositions

The present disclosure features a method of manufacturing or making a composition (e.g., an Active Moiety) of the foregoing invention. Amino acid entities used to make the compositions may be agglomerated, and/or instantized to aid in dispersal and/or solubilization.

The compositions may be made using amino acid entities from the following sources, or other sources may used: e.g., FUSI-BCAA™ Instantized Blend (L-Leucine, L-Isoleucine and L-Valine in 2:1:1 weight ratio), instantized L-Leucine, and other acids may be obtained from Ajinomoto Co., Inc. Pharma. grade amino acid entity raw materials may be used in the manufacture of pharmaceutical amino acid entity products. Food (or supplement) grade amino acid entity raw materials may be used in the manufacture of dietary amino acid entity products.

To produce the compositions of the instant disclosure, the following general steps may be used: the starting materials (individual amino acid entities and excipients) may be blended in a blending unit, followed by verification of blend uniformity and amino acid entity content, and filling of the blended powder into stick packs or other unit dosage form. The content of stick packs or other unit dosage forms may be dispersed in water at time of use for oral administration.

Food supplement and medical nutrition compositions of the invention will be in a form suitable for oral administration.

When combining raw materials, e.g., pharmaceutical grade amino acid entities and/or excipients, into a composition, contaminants may be present in the composition. A composition meets a standard for level of contamination when the composition does not substantially comprise (e.g., comprises less than 10, 9, 8, 7, 6, 5, 4, 3, 2, 1, 0.1, 0.01, or 0.001% (w/w)) a contaminant. In some embodiments, a composition described in a method herein does not comprise a contaminant. Contaminants include any substance that is not deliberately present in the composition (for example, pharmaceutical grade amino acid entities and excipients, e.g., oral administration components, may be deliberately present) or any substance that has a negative effect on a product quality parameter of the composition (e.g., side effects in a subject, decreased potency, decreased stability/shelf life, discoloration, odor, bad taste, bad texture/mouthfeel, or increased segregation of components of the composition). In some embodiments, contaminants include microbes, endotoxins, metals, or a combination thereof. In some embodiments, the level of contamination, e.g., by metals, lecithin, choline, endotoxin, microbes, or other contaminants (e.g., contaminants from raw materials) of each portion of a composition is below the level permitted in food.

### Excipients

The amino acid compositions of the present disclosure may be compounded or formulated with one or more excipients. Non-limiting examples of suitable excipients include a tastant, a flavorant, a buffering agent, a preservative, a stabilizer, a binder, a compaction agent, a lubricant, a dispersion enhancer, a disintegration agent, a flavoring agent, a sweetener, and a coloring agent.

In some embodiments, the excipient comprises a buffering agent. Non-limiting examples of suitable buffering agents include citric acid, sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the composition comprises a dispersion enhancer as an excipient. Non-limiting examples of suitable dispersants include starch, alginic acid, polyvinylpyrrolidones, guar gum, kaolin, xanthan gum, bentonite, purified wood cellulose, sodium starch glycolate, isoamorphous silicate, and microcrystalline cellulose as high HLB emulsifier surfactants.

In some embodiments, the composition comprises a disintegrant as an excipient. In some embodiments, the disintegrant is a non-effervescent disintegrant. Non-limiting examples of suitable non-effervescent disintegrants include starches such as corn starch, potato starch, pregelatinized and modified starches thereof, sweeteners, clays, such as bentonite, microcrystalline cellulose, alginates, sodium starch glycolate, gums such as agar, guar, locust bean, karaya, pecitin, and tragacanth. In some embodiments, the disintegrant is an effervescent disintegrant. Non-limiting examples of suitable effervescent disintegrants include sodium bicarbonate in combination with citric acid, and sodium bicarbonate in combination with tartaric acid.

In some embodiments, the excipient comprises a flavoring agent. Flavoring agents can be chosen from synthetic flavor oils and flavoring aromatics; natural oils; extracts from plants, leaves, flowers, and fruits; and combinations thereof. In some embodiments, the flavoring agent is selected from cinnamon oils; oil of wintergreen; peppermint oils; clover oil; hay oil; anise oil; Eucalyptus; vanilla; citrus oil such as lemon oil, orange oil, grape and grapefruit oil; and fruit essences including apple, peach, pear, strawberry, raspberry, cherry, plum, pineapple, and apricot.

In some embodiments, the excipient comprises a sweetener. Non-limiting examples of suitable sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, xylitol, and the like. Also contemplated are hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

In some embodiments, the composition comprises a coloring agent. Non-limiting examples of suitable color agents include food, drug and cosmetic colors (FD&C), drug and cosmetic colors (D&C), and external drug and cosmetic colors (Ext. D&C). The coloring agents can be used as dyes or their corresponding lakes.

Particular excipients may include one or more of: citric acid, lecithin, (e.g. Alcolec F100), sweeteners (e.g. sucralose, sucralose micronized NF, acesulfame potassium (e.g. Ace-K)), a dispersion enhancer (e.g. xanthan gum (e.g. Ticaxan Rapid-3)), flavorings (e.g. vanilla custard #4306, Nat Orange WONF #1326, lime 865.0032U, and lemon 862.2169U), a bitterness masking agent (e.g. 936.2160U), and natural or artificial colorings (e.g. FD&C Yellow 6). Exemplary ingredient contents for each stick pack are shown in Table 9.

TABLE 9
Ingredient contents in each stick pack.

| | | | SOURCE; |
|---|---|---|---|
| INGREDIENT | GRADE | FUNCTION | COMMENT |
| Amino | USP | Active | Various sources; |
| Acids | | Pharmaceutical | Non-instantized |
| | | Ingredient | form (MFG |
| | | (API) | scale) |
| Citric | USP | pH, Flavor | Spectrum Chems; |
| Acid | | | f(volume) ≤ 1.0% w/v |
| Acesulfame K | NF | Sweetness | Spectrum Chems; |
| | | (rapid onset) | Target 1 Sweetener |
| Sucralose | NF | Sweetness | Spectrum Chems; |
| | | (slow onset) | WHO ADI ≤ |
| | | | 15 mg/kg |
| Lecithin | FCC | Wetting | American Lecithin |
| (Alecolec F100) | | Agent | Company |
| Xanthan Gum | FCC | Stabilizer/ | TIC Gums; |
| | | Thickener | f(volume) ≤ 0.5% w/v |
| Vanilla | GRAS | Taste/Aroma | David Michael; |
| Custard (Art) | | | Mask sulfur |
| Orange (Natural | GRAS | 1° flavor | David Michael; Citrus |
| and WONF) | | | profile matches low pH |
| Lime (Natural | GRAS | 2° flavor | FONA; Single flavor |
| and WONF) | | | supplier |
| Lemon (Natural | GRAS | 2° flavor | FONA; Single flavor |
| and artificial) | | | supplier |
| Taste | GRAS | Bitterness | FONA; Useful at low |
| Modifier | | masking | volume |
| FD&C Yellow | USP | Color | Sensient; Match |
| No.6 | | | flavor profile |

In another embodiment, excipients are limited to citric acid, a sweetener (e.g., sucralose), xanthan gum, an aroma agent (e.g., vanilla custard #4036), a flavoring agent (e.g., Nat orange WONF #1362), and a coloring agent (e.g., FD&C Yellow 6), e.g., the excipient specifically excludes lecithin (Table 10).

TABLE 10
Exemplary contents in each stick pack.

| INGREDIENT | GRADE | FUNCTION |
|---|---|---|
| Amino | USP | Active Pharmaceutical |
| Acids | | Ingredient (API) |
| Citric Acid | USP | pH, Flavor |
| Sucralose | NF | Sweetness (slow onset) |
| Xanthan Gum | FCC | Stabilizer/Thickener |
| Vanilla Custard (Art) | GRAS | Aroma |
| Orange (Nat + WONF) | GRAS | 1° flavor |
| FD&C Yellow No. 6 | USP | Color |

### Dietary Compositions

The composition (e.g., Active Moiety) including amino acid entities can be formulated and used as a dietary composition, e.g., chosen from a medical food, a functional food, or a supplement. In such an embodiment, the raw materials and final product should meet the standards of a food product.

The composition of any of the aspects and embodiments disclosed herein can be for use as a dietary composition, e.g., chosen from a medical food, a functional food, or a supplement. In some embodiments, the dietary composition is for use in a method, comprising administering the composition to a subject. The composition can be for use in a dietary composition for the purpose of improving or reducing insulin resistance.

In some embodiments, the dietary composition is chosen from a medical food, a functional food, or a supplement. In some embodiments, the composition is in the form of a nutritional supplement, a dietary formulation, a functional food, a medical food, a food, or a beverage comprising a composition described herein. In some embodiments, the nutritional supplement, the dietary formulation, the functional food, the medical food, the food, or the beverage comprising a composition described herein for use in the management of insulin resistance or glucose tolerance (e.g., in a subject with insulin resistance).

The present disclosure features a method of improving insulin resistance or glucose tolerance comprising administering to a subject an effective amount of a dietary composition described herein.

The present disclosure features a method of providing nutritional support or supplementation to a subject with insulin resistance (e.g., a subject with a condition or disorder described herein), comprising administering to the subject an effective amount of a composition described herein.

The present disclosure features a method of providing nutritional support or supplementation that aids in the management of insulin resistance, comprising administering to a subject in need thereof an effective amount of a composition described herein.

In some embodiments, the subject has or has been diagnosed with insulin resistance. In other embodiments, the subject does not have insulin resistance.

Additionally, the compositions can be used in methods of dietary management of a subject (e.g., a subject without insulin resistance).

In some embodiments, the subject has prediabetes, Type 2 diabetes, or metabolic syndrome (Syndrome X). In some embodiments, the subject is overweight or obese.

In some embodiments, the subject has a cardiovascular condition or disorder. In certain embodiments, the subject has hypertension, dyslipidemia, atherosclerosis, or obstructive sleep apnea.

In some embodiments, the subject has an endocrine disorder. In certain embodiments, the endocrine disorder is chosen from: polycystic ovarian syndrome (PCOS), hyperthyroidism, Cushing's disease, Cushing's syndrome, acromegaly, or pheochromocytoma.

In some embodiments, the subject is pregnant.

In some embodiments, the subject has renal failure.

In some embodiments, the subject has a genetic condition or disorder. In certain embodiments, the subject has a genetic condition or disorder chosen from: Down's syndrome, Turner's syndrome, Klinefelter's syndrome, thalassaemia, haemochromatosis, lipodystrophy, progeria, Huntington's chorea, myotonic dystrophy, Friedrich's ataxia, Laurence-Moon-Biedl syndrome, glycogen storage diseases type I & III, or an inherited mitochondrial disorder.

In some embodiments, the subject has a cancer. In certain embodiments, the cancer is chosen from: colon, endometrial cancer, pancreatic cancer, renal-cell cancer, or breast cancer.

In some embodiments, the subject has dementia. In certain embodiments, the dementia is chosen from: Alzheimer's disease or Lewy body dementia.

In some embodiments, the subject has a syndrome of severe insulin resistance (SSIR). In certain embodiments, the SSIR is associated with lipodystrophy.

In some embodiments, the subject has a genetic disorder of insulin resistance. In certain embodiments, the genetic disorder of insulin resistance is chosen from: Donohue Syndrome, Rabson-Mendenhall Syndrome, or Type A Insulin Resistance.

### Biomarkers

Any of the methods disclosed herein can include evaluating or monitoring the effectiveness of administering a composition of the invention as described herein (e.g., the Active Moiety) to a subject with insulin resistance (e.g., a subject with a insulin resistance). The method includes acquiring a value of effectiveness to the composition, such that the value is indicative of the effectiveness of the therapy.

In some embodiments, the subject exhibits increased levels of proC3, e.g., relative to a healthy subject without insulin resistance. In some embodiments, the subject exhibits increased levels of ALT, e.g., relative to a healthy subject without insulin resistance. In some embodiments, the subject exhibits increased levels of AST, e.g., relative to a healthy subject without insulin resistance. In some embodiments, the subject exhibits increased levels of TIMP (e.g., TIMP1 or TIMP2), e.g., relative to a healthy subject without insulin resistance. In some embodiments, the subject exhibits increased levels of Co11a1, e.g., relative to a healthy subject without insulin resistance. In some embodiments, the subject exhibits increased levels of Acta2, e.g., relative to a healthy subject without insulin resistance. In some embodiments, the subject exhibits increased levels of hydroxyproline, e.g., relative to a healthy subject without insulin resistance.

In some embodiments, administration of the composition (e.g., the Active Moiety) at a dosage regimine described herein to the subject reduces the level or activity of one, two, three, four, five, six, seven, eight, nine, ten, 11, 12, 13, or more (e.g., all) of the following: (a) N-terminal fragment of type III collagen (proC3); (b) a tissue inhibitor of metalloproteinase (TIMP) protein; e.g., TIMP1 or TIMP2; (c) Co11a1; (d) Acta2; (e) ALT; (f) AST; (g) hydroxyproline; (h) TGF-b; (i) MCP-1; (j) MIP-1; (k) collagen, e.g., type I and III collagen; (1) α-smooth muscle actin (αSMA); (m) PIIINP; or (o) triglycerides.

### EXAMPLES

The Examples below are set forth to aid in the understanding of the inventions, but is not intended to, and should not be construed to, limit its scope in any way.

### Example 1. Therapeutic Treatment with Amino Acid Composition A-1 Improves Oral Glucose Tolerance in a Pre-Clinical Animal Model

Amino Acid Composition A-1 and metformin were tested for their ability to affect glucose tolerance in a genetically obese B6.Cg-Lep^{ob}/J (ob/ob) mouse model (Maida A, et al., 2010, PMID:20972533).

Model Description

B6.Cg-Lep^{ob}/J (ob/ob) mice harbor a spontaneous mutation of leptin (Lep) gene. ob/ob mice exhibit hyperphagia, obesity, and metabolic syndrome/T2DM-like symptoms, e.g. hyperglycemia, hyperinsulinemia, and insulin resistance. ob/ob mice have impaired intestinal barrier function, gut microbial translocation, and an inflammatory, fibrogenic phenotype of hepatic stellate cells (Brun P et al., 2004, PMID:17023554). ob/ob mice develop skeletal muscle hypoplasia in quadriceps femoris, similar to the effect of aging in humans (Hamrick M W et al., 2004, PMID:15003785). ob/ob mice exhibit intolerance to glucose and insulin. Metformin lowers plasma glucose (Cool B, et al., Cell Metab 2006, PMID:16753576), liver triglyceride, and reverses NAFLD in ob/ob mice (Lin H Z et al., 2000, PMID:10973319; Cool B, et al., Cell Metab 2006, PMID:16753576). A single dose of metformin treatment reduces blood glucose and improves glucose tolerance (OGTT) in C57/BL6.

Experimental Design

Eight-week-old male ob/ob mice were subjected to treatment of test articles (Amino Acid Composition A-1 and metformin) followed by oral glucose tolerance test (OGTT) on Day 3. Mice were randomized by body weight and unfasted blood glucose on Day −1. Body weight was recorded daily in the morning before AM dosing on Day 1, Day 2, and Day 3. Test articles were dosed by oral gavage at 10 ml/kg. Dosage of a test article was calculated based on daily body weight. Treatment schedule and dose are listed in the following section (Table 34). AM doses were administered at 0700, and PM doses were administered at 1800. Oral glucose tolerance test (OGTT) was performed after 6-hour fasting on Day 3.

TABLE 34
Treatment schedule

| Group | Test article | # | Dosing Schedule |
|---|---|---|---|
| Group | Vehicle | N = 5 | Vehicle dosed on Day 1 and Day 2 at 0700 and 1800, and |
| 1 | | | Day 3 at 0700 and 30 min before OGTT for a total of 6 |
| | | | doses. |
| Group | Metformin | N = 5 | Metformin hydrochloride (450 mg/kg, QD PO at the |
| 2 | | | beginning of dark cycle) dosed on Day −1, Day 1, and Day 2 |
| | | | at 1800, and at 30 min before OGTT on Day 3 for a total of 4 |
| | | | doses. |
| Group | Amino Acid | N = 5 | Amino Acid Composition A-l (1500 mg/kg, BID PO at |
| 3 | Composition | | 0700 and 1800) dosed on Day 1 and Day 2 at 0700 and 1800, |
| | A-1 | | and Day 3 at 0700 and 30 min before OGTT for a total of 6 |
| | | | doses. |
| Group | Amino Acid | N = 5 | Amino Acid Composition A-l (3000 mg/kg, BID PO at |
| 4 | Composition | | 0700 and 1800) dosed on Day 1 and Day 2 at 0700 and 1800, |
| | A-1 | | and Day 3 at 0700 and 30 min before OGTT for a total of 6 |
| | | | doses. |

Baseline Glucose and Biochemistry (Insulin, Triglyceride and Cholesterol)

Mice were fasted for 6 hours prior to OGTT test. Food was removed at 0700 hours on Day 3; water was provided during fasting. Blood samples were collected from tail snip or facial puncture at −30 min (relative to OGTT) into K2EDTA tubes for baseline glucose and blood biochemistry (insulin, triglyceride, and cholesterol). Blood glucose was measured by a glucometer (SDI StatStrip Xpress or equivalent). Plasma was collected in K2EDTA and saved at −80° C.

Oral Glucose Tolerance Test (OGTT)

Mice were bled for baseline glucose and plasma at −30 min. Test articles were then dosed by oral gavage at −30 min. Glucose was administered per os (P.O.) at a dosage of 2.0 g/kg body weight. Blood glucose levels were measured at 0 min immediately prior to glucose injection and then at 15, 30, 60, 120 and 240 minutes thereafter (shown as 0.25, 0.5, 1, 2, and 4 hours in Table 35 below).

Results are shown in Table 35. Data are mean±standard deviation (stdev). (p values by Dunnett's multiple comparisons: **p<0.005 compared to vehicle control; ***p<0.001 compared to vehicle control; ****p<0.0005 compared to vehicle control.)

### Results

TABLE 35
OGTT results: Mean Blood glucose levels (mg/dl) and standard deviations (stdev)

| | | | Amino Acid | | Amino Acid | | | |
|---|---|---|---|---|---|---|---|---|
| | | | Comp A-1, | | Comp A-1, | | | |
| | Vehicle | | 1500 mg/kg | | 3000 mg/kg | | Metformin | |
| | mean | | mean | | mean | | mean | |
| | blood | | blood | | blood | | blood | |
| | glucose | | glucose | | glucose | | glucose | |
| Timepoint | level | | level | | level | | level | |
| (hours) | (mg/dl) | stdev | (mg/dl) | stdev | (mg/dl) | stdev | (mg/dl) | stdev |
| −1 | 241.8 | 108.3 | 245.6 | 89.4 | 229.6 | 78.2 | 196.4 | 59.8 |
| 0 | 282.6 | 47.0 | 374.6 | 97.6 | 303.0 | 77.1 | 199.4 | 62.6 |
| 0.25 | 655.0 | 107.2 | 575.6 | 73.8 | 456.2** | 36.6 | 353.6**** | 73.6 |
| 0.5 | 640.6 | 92.6 | 555.2 | 84.0 | 513.0 | 47.9 | 390.2*** | 99.5 |
| 1 | 378.0 | 111.1 | 386.6 | 27.5 | 316.4 | 86.1 | 317.6 | 116.9 |
| 2 | 236.6 | 54.8 | 243.5 | 18.4 | 230.0 | 101.1 | 158.2 | 44.0 |
| 4 | 197.8 | 53.3 | 214.8 | 56.8 | 179.8 | 81.3 | 109.4 | 29.0 |

### SUMMARY

Treatment with Amino Acid Composition A-1 resulted in improvement of oral glucose tolerance, as indicated by improved blood glucose clearance upon oral glucose loading. In addition, 3-day treatment with Amino Acid Composition A-1 did not alter baseline blood glucose in ob/ob mice (Table 36).

TABLE 36
OGTT results: Blood glucose levels (mg/dl) raw data

| Timepoint | | | | | |
|---|---|---|---|---|---|
| (hours) | Vehicle | | | | |
| −1 | 211 | 141 | 211 | 219 | 427 |
| 0 | 239 | 256 | 273 | 284 | 361 |
| 0.25 | 741 | 676 | 514 | 578 | 766 |
| 0.5 | 551 | 621 | 604 | 630 | 797 |
| 1 | 305 | 317 | 327 | 369 | 572 |
| 2 | 182 | 243 | 230 | 203 | 325 |
| 4 | 146 | 203 | 167 | 188 | 285 |

| Timepoint | | | | | |
|---|---|---|---|---|---|
| (hours) | Amino Acid Comp A-1, 1500 mg/kg | | | | |
| −1 | 224 | 220 | 190 | 191 | 403 |
| 0 | 331 | 548 | 347 | 316 | 331 |
| 0.25 | 526 | 702 | 580 | 531 | 539 |
| 0.5 | 532 | 419 | 621 | 587 | 617 |
| 1 | 431 | 365 | 367 | 395 | 375 |
| 2 | 246 | | 243 | 220 | 265 |
| 4 | 192 | | 193 | 175 | 299 |
| Timepoint | | | | | |
| (hours) | Amino Acid Comp A-1, 3000 mg/kg | | | | |
| −1 | 137 | 294 | 179 | 214 | 324 |
| 0 | 242 | 359 | 203 | 329 | 382 |
| 0.25 | 412 | 490 | 438 | 443 | 498 |
| 0.5 | 513 | 482 | 467 | 512 | 591 |
| 1 | 240 | 351 | 220 | 342 | 429 |
| 2 | 161 | 334 | 148 | 160 | 347 |
| 4 | 120 | 235 | 110 | 139 | 295 |

| Timepoint | | | | | |
|---|---|---|---|---|---|
| (hours) | Metformin | | | | |
| −1 | 265 | 248 | 183 | 120 | 166 |
| 0 | 274 | 234 | 220 | 129 | 140 |
| 0.25 | 387 | 397 | 427 | 310 | 247 |
| 0.5 | 462 | 439 | 482 | 307 | 261 |
| 1 | 365 | 399 | 431 | 239 | 154 |
| 2 | 154 | 183 | 219 | 124 | 111 |
| 4 | 118 | 93 | 155 | 101 | 80 |

### Example 2. Therapeutic Treatment of NAFLD, NASH, and HCC with Amino Acid Composition A-1 in a Pre-Clinical Animal Model

Amino Acid Composition A-1 and Obeticholic acid (6α-ethyl-chenodeoxycholic acid; “OCA”) were tested for their ability to treat NASH in the STAM™ model (Stelic Institute & Co., Tokyo, Japan; Saito K. et al., 2015 Sci Rep 5: 12466). Two additional groups of normal C57BL/6 mice fed standard chow and vehicle treated STAM™ mice were included as controls. All animals receiving treatment or vehicle were treated starting at 6 weeks until 9 weeks of age. Compounds were administered via oral gavage, with a dose volume of 10 ml/kg. Amino Acid Composition A-1 was administered twice daily at a dose of 1500 mg/kg, and OCA was administered once daily at a dose of 30 mg/kg.

STAM™ Mouse Model Description

STAM™ is a model for non-alcoholic steatohepatitis (NASH) and hepatocellular carcinoma (HCC), developed by SMC Laboratories, Inc. and created by the combination of chemical and dietary interventions using C57BL/6 mice (Saito K. et al., 2015 Sci Rep 5: 12466). Mice are treated with a low dose of streptozotocin at birth and fed a high fat diet starting at 4 weeks. Evidence of fatty liver is present by 5 weeks, followed by NASH by 7 weeks and fibrosis by 9 weeks.

Induction of NASH

NASH was induced in 53 male mice by a single subcutaneous injection of 200 μg streptozotocin (STZ, Sigma-Aldrich, USA) solution 2 days after birth and feeding with high fat diet (HFD, 57 kcal % fat, Cat #HFD32, CLEA Japan, Japan) after 4 weeks of age.

Route of Drug Administration, Preparation of Dosing Solutions and Treatment Doses

Amino Acid Composition A-1, OCA and Vehicle (described below) were administered by oral route in a volume of 10 mL/kg. Amino Acid Composition A-1 was solubilized in deionized water to 150 mg/ml (10×). OCA (Advanced ChemBlocks Inc.) was resuspended in 0.5% methycellulose in water to 3 mg/ml (10×). Amino Acid Composition A-1 was administered at a dose of 1500 mg/kg twice daily (9 am and 7 pm). OCA was administered at a dose of 30 mg/kg once daily (9 am).

Histological Analyses

Liver samples from mice in Group 2 (Vehicle), 3 (Amino Acid Composition A-1) and 4 (OCA) were used for the following assays. For HE staining, sections were cut from paraffin blocks of liver tissue prefixed in Bouin's solution and stained with Lillie-Mayer's Hematoxylin (Muto Pure Chemicals Co., Ltd., Japan) and eosin solution (Wako Pure Chemical Industries). NAFLD Activity score (NAS) was calculated according to the criteria of Kleiner (Kleiner D. E. et al., Hepatology, 2005; 41:1313).

Experimental Design

Study Groups

Group 1: STZ: Ten neonatal STZ-primed mice were fed with a normal diet ad libitum without any treatment until 9 weeks of age.

Group 2: Vehicle: Ten NASH mice were orally administered vehicle (10% phosphate buffered saline, pH 7.2) in a volume of 10 mL/kg twice daily (9 am and 7 pm) from 6 to 9 weeks of age.

Group 3: Amino Acid Composition A-1: Ten NASH mice were orally administered water for irrigation supplemented with Amino Acid Composition A-1 at a dose of 1500 mg/kg twice daily (9 am and 7 pm) from 6 to 9 weeks of age.

Group 4: OCA: Ten NASH mice were orally administered 0.5% methylcellulose supplemented with OCA at a dose of 30 mg/kg once daily (9 am) from 6 to 9 weeks of age.

Group 5: Normal: Ten normal mice were fed with a normal diet ad libitum without any treatment until 9 weeks of age.

Group 6: HFD: Ten normal mice were fed with a high fat diet ad libitum without any treatment until 9 weeks of age.

### Histological Results: HE Staining, NAFLD Activity Score and α-Smooth Muscle Actin Staining

Non-Alcoholic Fatty Liver Disease Activity Score Results

The non-alcoholic fatty liver disease (NAFLD) activity score was assessed via histological analysis and grading of H&E stained liver sections from each animal. This score is the sum of three individual scores that grade the degree of steatosis (0-3), inflammation (0-2), and hepatocyte ballooning (0-2). All tissues were graded using the scoring criteria of Kleiner et al. (Kleiner et al. Hepatology. 2005; 41(6):1313-21). Results are shown in Table 37. Data are mean±standard deviation (stdev). Normal C57BL/6 mice fed standard chow had a mean score of 0+/−0. Vehicle treated STAM™ mice had a mean score of 4.7+/−0.67. Amino Acid Composition A-1 treated mice had a mean score of 3.1+/−0.74. OCA treated mice had a mean score of 2.9+/−0.74. Both Amino Acid Composition A-1 and OCA were statistically different from vehicle for NAFLD Activity Score when compared using Dunnett's multiple comparisons test (Amino Acid Composition A-1 p=0.0001, OCA p=0.0001).

Similarly, Amino Acid Composition A-1 treated mice showed a mean ballooning score of 0.4+/−0.52, compared to a mean ballooning score for vehicle treated STAM™ mice of 1.6+/−0.52, and a mean ballooning score for OCA treated mice of 0.3+/−0.48. Both Amino Acid Composition A-1 and OCA were statistically different from vehicle for ballooning score when compared using Dunnett's multiple comparisons test (Amino Acid Composition A-1 p=0.0001, OCA p=0.0001). Raw data are shown in Tables 37-40.

TABLE 37
NAFLD Activity Score
NAFLD Activity Score (NAS)

| | Normal | Vehicle- | Amino Acid | OCA |
|---|---|---|---|---|
| | C57BL/6 | treated | Composition A-1 | treated |
| Condition | mice | STAM mice | treated STAM mice | STAM mice |
| Mean | 0 | 4.7 | 3.1 | 2.9 |
| stdev | 0 | 0.67 | 0.74 | 0.74 |

TABLE 38
NAFLD Activity: Steatosis Score

| | Steatosis | | | |
|---|---|---|---|---|
| | Normal | Vehicle- | Amino Acid | OCA |
| | C57BL/6 | treated | Composition A-1 | treated |
| | mice | STAM mice | treated STAM mice | STAM mice |
| Mean | 0 | 1 | 0.9 | 0.8 |
| stdev | 0 | 0.00 | 0.32 | 0.42 |

TABLE 39
NAFLD Activity: Inflammation Score

| | Inflammation | | | |
|---|---|---|---|---|
| | Normal | Vehicle- | Amino Acid | OCA |
| | C57BL/6 | treated | Composition A-1 | treated |
| | mice | STAM mice | treated STAM mice | STAM mice |
| Mean | 0 | 2.1 | 1.8 | 1.8 |
| stdev | 0 | 0.32 | 0.63 | 0.79 |

TABLE 40
NAFLD Activity: Ballooning Score

| | Ballooning | | | |
|---|---|---|---|---|
| | Normal | Vehicle- | Amino Acid | OCA |
| | C57BL/6 | treated | Composition A-1 | treated |
| | mice | STAM mice | treated STAM mice | STAM mice |
| Mean | 0 | 1.6 | 0.4 | 0.3 |
| stdev | 0 | 0.52 | 0.52 | 0.48 |

Fibrosis: Sirius Red Staining Results

Fibrosis was assessed by analysis of Sirius red positively stained cell area from stained liver sections from each animal. Images were quantified using the percent of positively stained area was used as a measure of fibrosis. Results of this analysis are shown in Table 39. Data are mean±standard deviation (stdev). Normal C57BL/6 mice fed standard chow had a mean positive area of 0.286+/−0.09. Vehicle treated STAM™ mice had a mean positive area of 1.1+/−0.26. Amino Acid Composition A-1 treated mice had a mean positive area of 0.828+/−0.33. OCA treated mice had a mean score of 0.776+/−0.25. Amino Acid Composition A-1 and OCA were statistically different from vehicle when compared using Dunnett's multiple comparisons test (Amino Acid Composition A-1 p=0.00494, OCA p<0.016). Raw data are shown in Table 41.

TABLE 41
Fibrosis (mean positively stained area, Sirius red)

| | Normal | Vehicle- | Amino Acid | OCA |
|---|---|---|---|---|
| | C57BL/6 | treated | Composition A-1 | treated |
| Condition | mice | STAM mice | treated STAM mice | STAM mice |
| Mean | 0.286 | 1.1 | 0.828 | 0.776 |
| stdev | 0.09 | 0.26 | 0.33 | 0.25 |

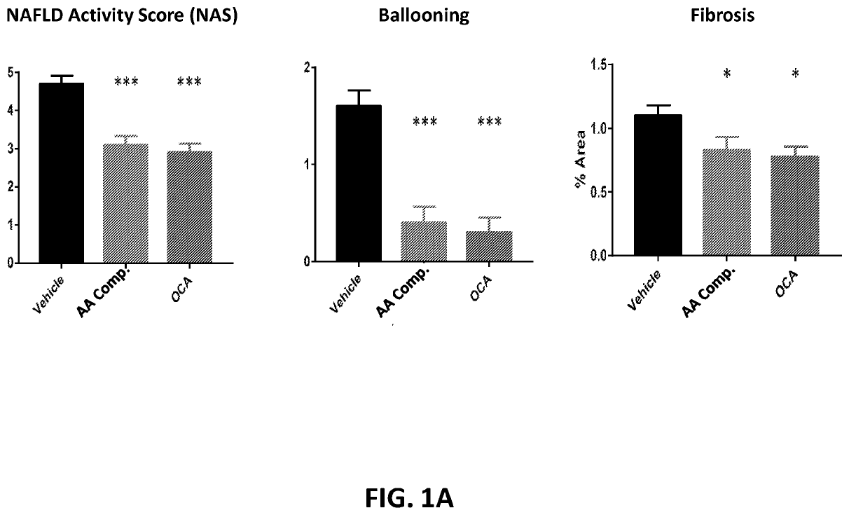

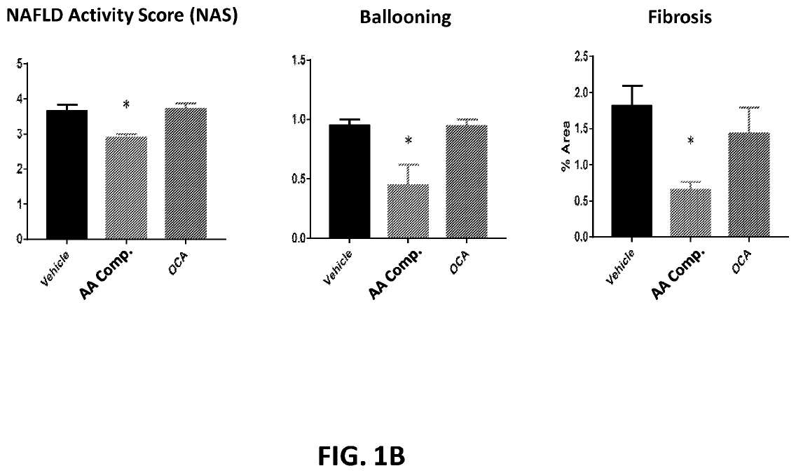

Similarly to the statistically significant improvement in the NAFLD activity score, ballooning, and fibrosis in the STAM mouse model after treatment with Amino Acid Composition A-1 (FIG. 1A), a statistically significant improvement in the NAFLD activity score, ballooning, and fibrosis was determined in the high-fat, high fructose and cholesterol diet (HFFC) mouse model after treatment with Amino Acid Composition A-1 (FIG. 1B).

α-Smooth Muscle Actin (α-SMA) Staining Results

Liver sections of all mice were stained for the marker α-smooth muscle actin (αSMA) to identify activated hepatic stellate cells. Images were quantified using the percent of positively stained area was used as a measure of stellate cell activation. Results are shown in Table 42. Data are mean±standard deviation (stdev); p values are compared to vehicle-treated STAM mice control; by one-tailed T test.

Normal C57BL/6 mice fed standard chow had a mean positive area of 0.682+/−0.26. Vehicle treated STAM™ mice had a mean positive area of 2.128+/−0.50. Amino Acid Composition A-1 treated mice had a mean positive area of 1.657+/−0.84. OCA treated mice had a mean score of 1.562+/−0.31.

TABLE 42
Activated hepatic stellate cells (mean positively
stained area, α-smooth muscle actin)

| | Normal | Vehicle- | Amino Acid | OCA |
|---|---|---|---|---|
| | C57BL/6 | treated | Composition A-1 | treated |
| Condition | mice | STAM mice | treated STAM mice | STAM mice |
| Mean | 0.682 | 2.128 | 1.657 | 1.562 |
| stdev | 0.26 | 0.50 | 0.84 | 0.31 |
| | | | p = 0.073 | p < 0.05 |

### SUMMARY

Treatment with Amino Acid Composition A-1 significantly reduced NASH severity to levels equivalent to Farnesoid X Receptor (FXR) inhibition by OCA (which is currently under clinical investigation by Intercept Pharmaceuticals, Inc. for treatment of NASH), as indicated by significant reduction in NAFLD Activity Score (NAS) (mean NAS:3.1+/−0.74 for Amino Acid Composition A-1 vs. vehicle treated STAM™ mice mean score of 4.7+/−0.67, compared to OCA treated mice mean score of 2.9+/−0.74), and development of fibrosis as indicated by the downregulation of hepatic stellate cell activation (mean αSMA positively stained area:1.657+/−0.84 for Amino Acid Composition A-1 vs. vehicle treated STAM™ mice mean area of 2.128+/−0.50, compared to OCA treated mice mean area of 1.562+/−0.31).

TABLE 43
NAFLD Activity Score: raw data

| Normal | Vehicle- | Amino Acid | OCA |
|---|---|---|---|
| C57BL/6 | treated | Composition A-1 | treated |
| mice | STAM mice | treated STAM mice | STAM mice |
| 0 | 6 | 3 | 4 |
| 0 | 5 | 4 | 2 |
| 0 | 5 | 4 | 2 |
| 0 | 4 | 3 | 4 |
| 0 | 5 | 2 | 3 |
| 0 | 5 | 2 | 3 |
| 0 | 4 | 3 | 2 |
| 0 | 4 | 3 | 3 |
| 0 | 4 | 3 | 3 |
| 0 | 5 | 4 | 3 |

TABLE 44
NAFLD Activity: Steatosis Score: raw data
Steatosis

| Normal | Vehicle- | Amino Acid | OCA |
|---|---|---|---|
| C57BL/6 | treated | Composition A-1 | treated |
| mice | STAM mice | treated STAM mice | STAM mice |
| 0 | 1 | 1 | 1 |
| 0 | 1 | 1 | 1 |
| 0 | 1 | 1 | 1 |
| 0 | 1 | 1 | 1 |
| 0 | 1 | 0 | 1 |
| 0 | 1 | 1 | 0 |
| 0 | 1 | 1 | 1 |
| 0 | 1 | 1 | 0 |
| 0 | 1 | 1 | 1 |
| 0 | 1 | 1 | 1 |

TABLE 45
NAFLD Activity: Inflammation Score: raw data
Inflammation

| Normal | Vehicle- | Amino Acid | OCA |
|---|---|---|---|
| C57BL/6 | treated | Composition A-1 | treated |
| mice | STAM mice | treated STAM mice | STAM mice |
| 0 | 3 | 1 | 2 |
| 0 | 2 | 2 | 1 |
| 0 | 2 | 2 | 1 |
| 0 | 2 | 2 | 2 |
| 0 | 2 | 1 | 2 |
| 0 | 2 | 1 | 3 |
| 0 | 2 | 2 | 1 |
| 0 | 2 | 2 | 3 |
| 0 | 2 | 2 | 2 |
| 0 | 2 | 3 | 1 |

TABLE 46
NAFLD Activity: Ballooning Score: raw data
Ballooning

| Normal | Vehicle- | Amino Acid | OCA |
|---|---|---|---|
| C57BL/6 | treated | Composition A-1 | treated |
| mice | STAM mice | treated STAM mice | STAM mice |
| 0 | 2 | 1 | 1 |
| 0 | 2 | 1 | 0 |
| 0 | 2 | 1 | 0 |
| 0 | 1 | 0 | 1 |
| 0 | 2 | 1 | 0 |
| 0 | 2 | 0 | 0 |
| 0 | 1 | 0 | 0 |
| 0 | 1 | 0 | 0 |
| 0 | 1 | 0 | 0 |
| 0 | 2 | 0 | 1 |

TABLE 47
Fibrosis (mean positively stained area, Sirius red): raw data

| Normal | Vehicle- | Amino Acid | OCA |
|---|---|---|---|
| C57BL/6 | treated | Composition A-1 | treated |
| mice | STAM mice | treated STAM mice | STAM mice |
| 0.26 | 0.79 | 1.07 | 0.36 |
| 0.35 | 1.43 | 0.58 | 0.56 |
| 0.19 | 1.44 | 0.48 | 1.1 |
| 0.31 | 1.36 | 0.58 | 1.19 |
| 0.19 | 1.04 | 1.07 | 0.89 |
| 0.36 | 0.75 | 0.34 | 0.91 |
| 0.24 | 1.07 | 0.86 | 0.66 |
| 0.37 | 1.13 | 1.43 | 0.72 |
| 0.18 | 0.83 | 0.96 | 0.68 |
| 0.41 | 1.16 | 0.91 | 0.69 |

TABLE 48
Activated hepatic stellate cells (mean positively
stained area, α-smooth muscle actin): raw data

| Normal | Vehicle- | Amino Acid | OCA |
|---|---|---|---|
| C57BL/6 | treated | Composition A-1 | treated |
| mice | STAM mice | treated STAM mice | STAM mice |
| 0.47 | 2.16 | 0.81 | 1.46 |
| 0.59 | 2.77 | 1.35 | 1.51 |
| 1.13 | 2.21 | 1.3 | 1.49 |
| 0.52 | 1.5 | 3.03 | 1.17 |
| 0.75 | 2.87 | 2.04 | 1.49 |
| 0.46 | 1.93 | 0.97 | 1.5 |
| 0.37 | 1.6 | 3.08 | 1.13 |
| 0.85 | 1.46 | 1.91 | 2.03 |
| 0.62 | 2.36 | 1.15 | 1.87 |
| 1.06 | 2.42 | 0.93 | 1.97 |

### Example 3. Treatment with an Amino Acid Composition Ameliorates NASH Progression in Two Rodent Models by Impacting Lipid Metabolism, Inflammation, and Fibrosis

The amino acid composition is formulated to simultaneously target multiple mechanisms of disease pathology to safely and effectively treat NASH (Table 52). As described herein, the efficacy of the amino acid composition was studied in two established mouse models of NASH to determine the effect of the amino acid composition on signs and symptoms associated with NASH and related disorders.

TABLE 52
Exemplary amino acid components of the amino acid composition.

| Amino acid | wt. ratio | wt. % | g/packet | g dose #1 | | g dose #2 | |
|---|---|---|---|---|---|---|---|
| Leucine | 1 | 16.78 | 1.00 g | 2 | g | 4 | g |
| Isoleucine | 0.5 | 8.39 | 0.50 g | 1 | g | 2 | g |
| Valine | 0.5 | 8.39 | 0.50 g | 1 | g | 2 | g |
| Arginine HCl | 1.81 | 30.37 | 1.81 g | 3.62 | g | 7.24 | g |
| Glutamine | 2 | 33.56 | 2.00 g | 4 | g | 8 | g |
| N-acetylcysteine | 0.15 | 2.52 | 0.15 g | 0.3 | g | 0.6 | g |
| Total amino acids | | | 5.96 g | ~12 | g | ~24 | g |

Animal Studies

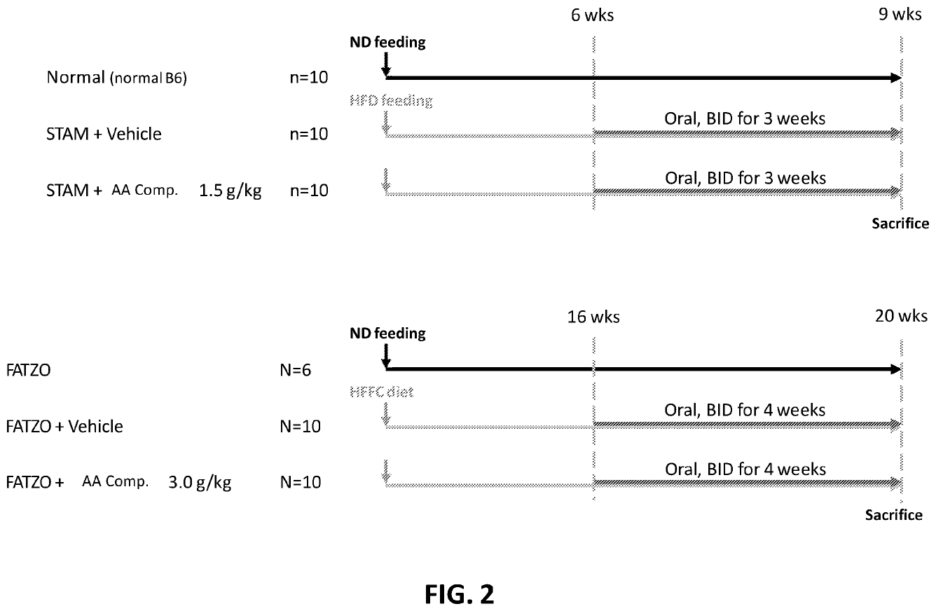

The STAM™ mouse is a model for non-alcoholic steatohepatitis (NASH) and hepatocellular carcinoma (HCC), developed by SMC Laboratories, Inc. Evidence of fatty liver is present by 5 weeks of age, followed by NASH by 7 weeks of age, and fibrosis by 9 weeks of age. Male STAM mice were generated in C57BL/6 mice, which received a low dose streptozotocin 2 days after birth and were fed a high fat diet (57% kcal fat, HFD32, CLEA Japan, Inc.) starting at 4 weeks old (Saito K. et al., 2015 Sci Rep 5: 12466; hereby incorporated by reference in its entirety). The amino acid composition was administered to STAM mice at a dose of 1.6 m/kg twice daily for 3 weeks starting at 6 weeks of age. One group of vehicle treated STAM mice was included as a control. Unfasted mice were euthanized at 9 weeks old. Plasma and liver samples were harvested for further analysis (FIG. 2).

The FATZO™ mouse is an inbred, polygenic model of obesity, metabolic syndrome, and NASH, developed by Crown Bioscience, Inc (Peterson R G. Et al., 2017 PLoS One; hereby incorporated by reference in its entirety). Male FATZO mice were fed a high fat, fructose, and cholesterol (HFFC) diet (40% kcal fat, D12079B, Research Diets, Inc. and 5% fructose in drinking water) starting at 6 weeks old to induce NAFLD and NASH. Evidence of fatty liver is present by 4 weeks post induction, followed by NASH by 16 weeks post induction and fibrosis by 20 weeks of induction. The designed amino acid composition was administered at a dose of 3.0 g/kg twice daily for 4 weeks starting at 16 weeks post induction (FIG. 2). One group of vehicle treated FATZO mice was included as control. Unfasted mice were euthanized at 20 weeks post-induction. Plasma and liver samples were harvested for further analysis.

Histological Analysis

The Aperio ScanScope CS whole slide digital imaging system (Vista, Calif.) was used for imaging in H&E, Picric Sirius Red, SMA, F4/80. Images were captured from whole slides.

The livers were evaluated by veterinary pathologists blind to sample ID using the NASH Clinical Research Network (CRN) liver histological scoring system (Kleiner D E, et al., 2015, hereby incorporated by reference in its entirety). The NASH CRN Scoring System assesses progression of steatosis, lobular inflammation, hepatocyte ballooning, degeneration, and fibrosis. One cross section of liver for each case was analyzed with the NASH score system. Steatosis, lobular inflammation, and fibrosis progression was assessed on a 0-3 scale. Ballooning degeneration was assessed on a 0-2 scale.

The Positive Pixel Count algorithm of the Aperio Automatic Image Quantitation was used to quantify the percentage of a specific stain present in a scanned slide image. A range of color (range of hues and saturation) and three intensity ranges (weak, positive, and strong) were masked and evaluated. The algorithm counted the number and intensity-sum in each intensity range, along with three additional quantities: average intensity, ratio of strong/total number, and average intensity of weak positive pixels.

A specific positive pixel algorithm was used for imaging the Sirius Red and Oil Red 0 liver sections. The positive pixel algorithm was modified to distinguish between the orange and blue colors. Alterations from the normal “hue value” (0.1 to 0.96) and “color saturation” (0.04 to 0.29), were made for the Sirius Red evaluation. Vasculature and artifacts were excluded from analysis.

Liver Triglyceride and Cholesterol Measurement

Liver total lipid-extracts were obtained by Folch's method (Folch J. et al., J. Biol. Chem. 1957; 226: 497; hereby incorporated by reference in its entirety). Liver samples were homogenized in chloroform-methanol (2:1, v/v) and incubated overnight at room temperature. After washing with chloroform-methanol-water (8:4:3, v/v/v), the extracts were evaporated to dryness, and dissolved in isopropanol. Liver triglyceride and cholesterol contents were measured by the Triglyceride E-test and Cholesterol E-test, respectively.

Gene Expression Analysis

Liver RNA samples were converted into cDNA libraries using the Illumina TruSeq Stranded mRNA sample preparation kit (Illumina #RS-122-2103). Transcriptome were analyzed at Q2 Solutions (Morrisville, N.C.). RNA Seq data were normalized and analyzed using Ingenuity Pathway Analysis (QIAGEN Bioinformatics). Mouse liver gene expression at the pathway level was focused on because it is translatable to human NAFLD (Teufel A, et al., Gastroenterology, 2016, hereby incorporated by reference in its entirety).

Metabolite Analysis

Metabolic profiling based on both capillary electrophoresis time-of-flight mass spectrometry (CE-TOFMS) and LC-TOFMS platforms was performed at Human Metabolome Technologies (Yamagata, Japan). Metabolites in the samples were identified by comparing the migration time and m/z ratio with authentic standards and quantified by comparing their peak areas with those of authentic standards.

Liver Cytokine/Chemokine Measurement

The levels of IL-1b, MCP-1, and MIP-1 protein in liver were quantified using the multiplex ELISA Assay (Meso Scale Discovery, Rockville, Md.).

The Amino Acid Composition Improves Ballooning and Fibrosis in Both STAM and FATZO Mice

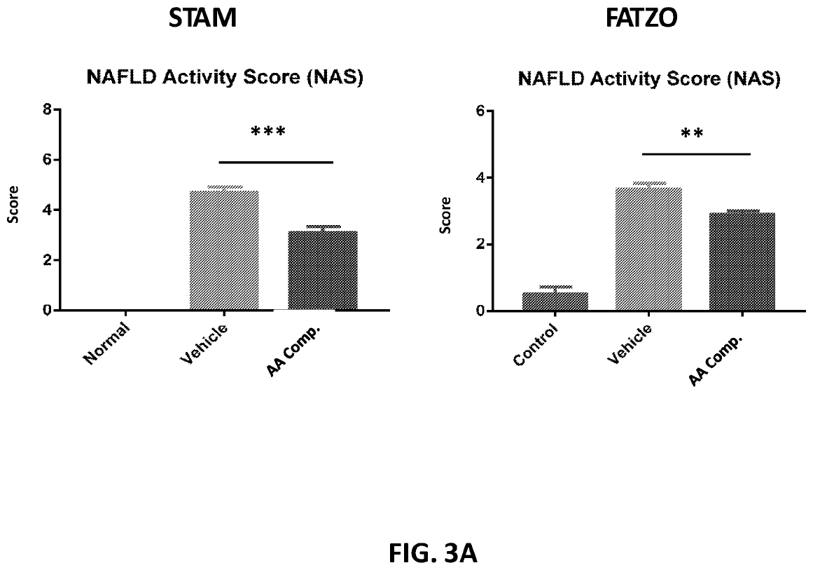

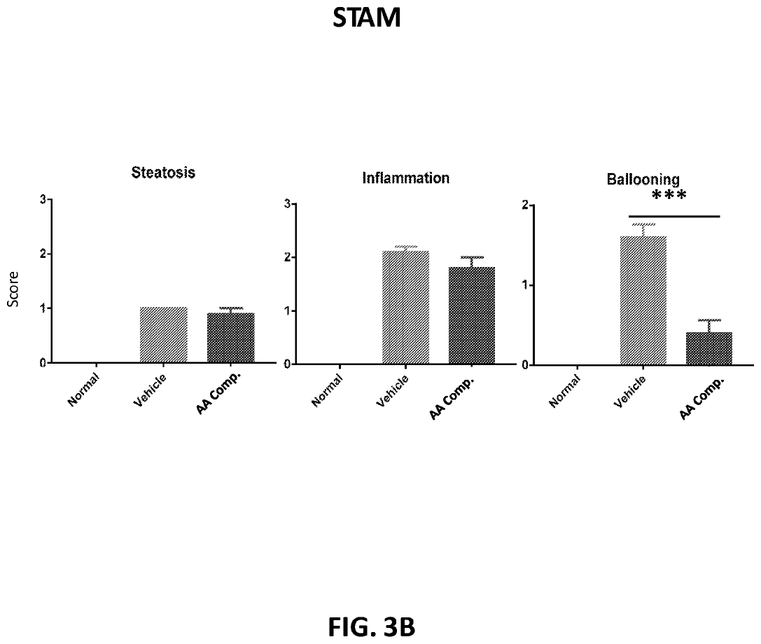

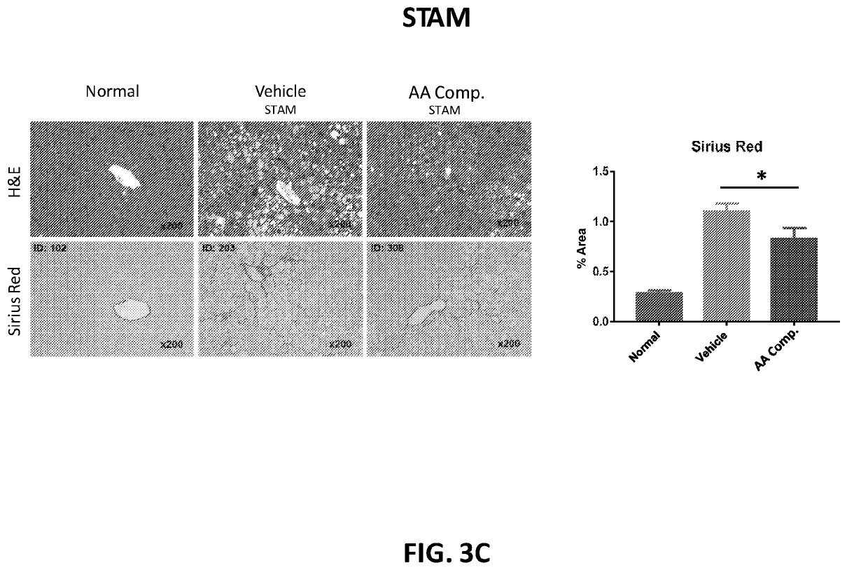

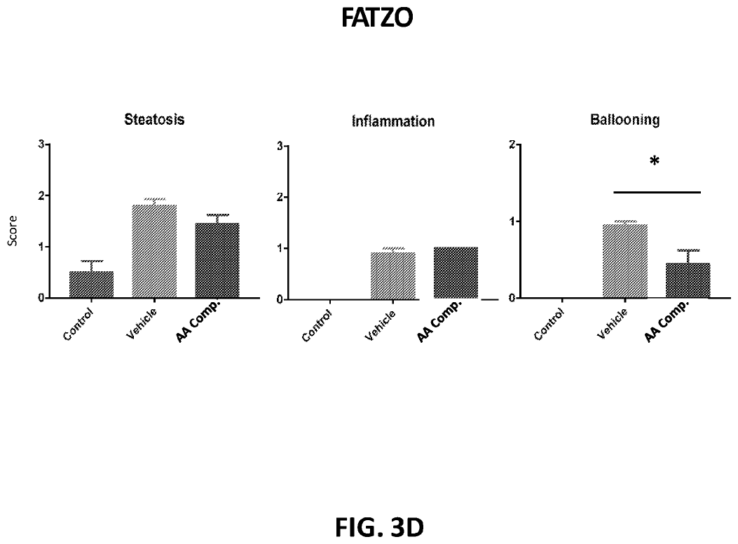

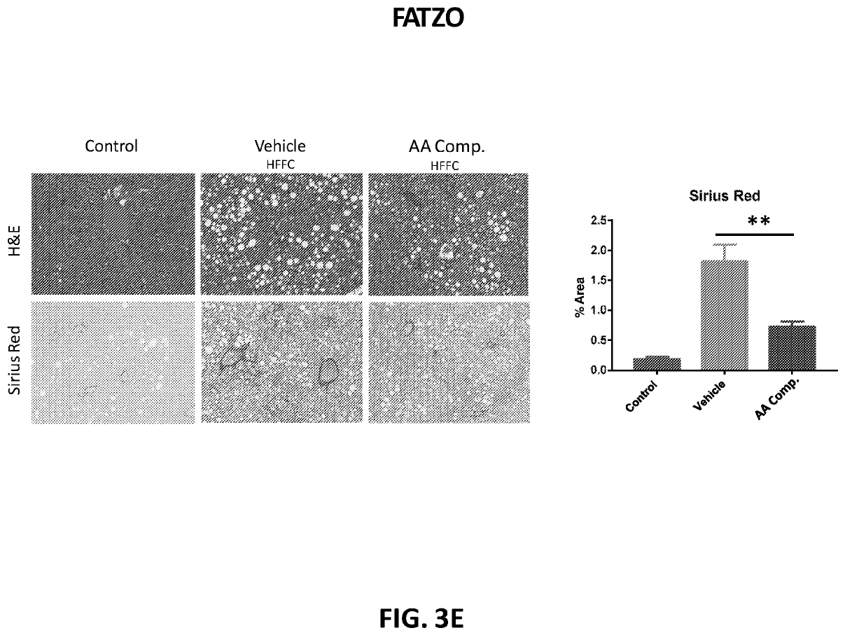

Treatment with the amino acid composition significantly reduced NAFLD activity scores (NAS) in both STAM and FATZO mice (FIG. 3A). Treatment with the amino acid composition also significantly decreased hepatocyte ballooning in STAM mice (FIG. 3B). Scores of steatosis and inflammation were not changed according to histological measures by treatment of STAM mice with the amino acid composition. The Sirius Red-positive, fibrosis area was significantly lowered by treating the STAM mice with the amino acid composition, while the Oil Red 0 area was not changed by treating the STAM mice with the amino acid composition (FIG. 3C). Liver triglyceride and cholesterol levels were not changed.

Treatment with the amino acid composition also significantly decreased hepatocyte ballooning in FATZO mice (FIG. 3D). Scores of steatosis and inflammation as well as liver triglyceride and cholesterol levels were not changed in the FATZO mice treated with the amino acid composition treatment. The Sirius Red-positive, fibrosis area was significantly lowered by treatment of the FATZO mice with the amino acid composition, while the Oil Red 0 area was not changed by treatment of the FATZO mice with the amino acid composition treatment (FIG. 3E).

The Amino Acid Composition Enhances Fatty Acid Oxidation

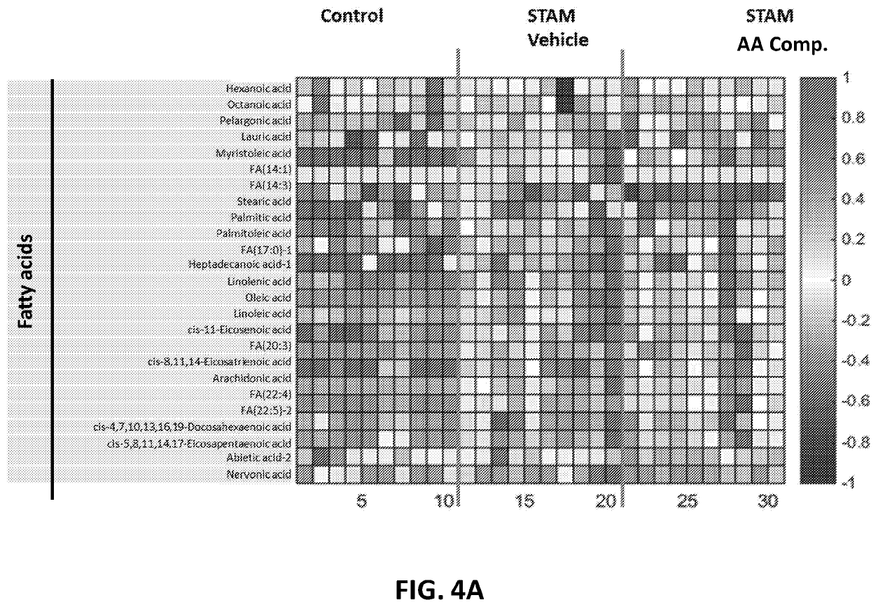

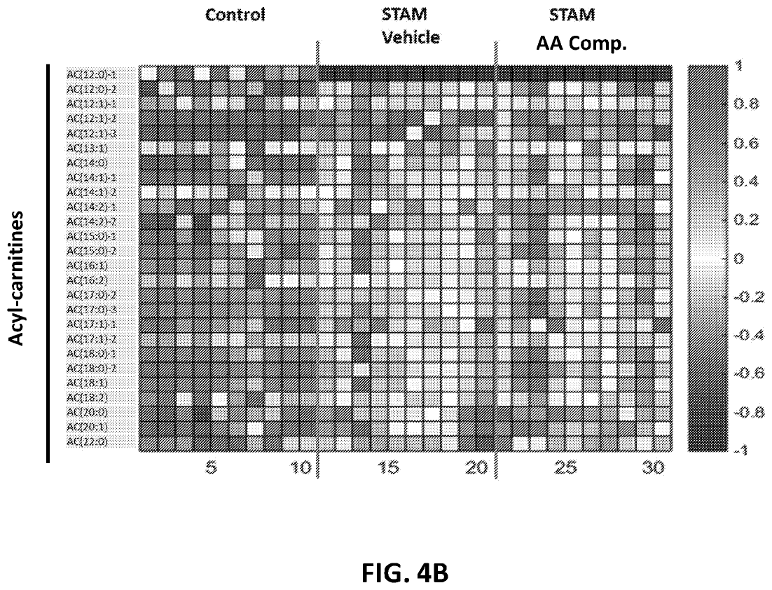

NAFLD is characterized by hepatic lipid accumulation. Liver triglyceride is attributable to a precise balance between acquisition by de novo lipogenesis and uptake of non-esterified fatty acids from the plasma, versus disposal by fatty acid oxidation and by the secretion of triglyceride-rich lipoproteins (Kawano Y, Cohen D E, J Gastroenterol. 2013, hereby incorporated by reference in its entirety). Compared to control mice, STAM mice had higher liver unsaturated fatty acids, which were reduced by treatment with the amino acid composition (FIG. 4A and Table 53). Liver acylcarnitines in STAM mice were increased by treatment with the amino acid composition, suggesting enhanced fatty acid beta-oxidation (FIG. 4B and Table 53).

TABLE 53
P-values and fold changes for liver acylcarnitine and unsaturated fatty acids following
treatment of STAM mice with the amino acid composition (treated) compared to control.

| | KEGG | HMDB | Control | Control | Treated | Treated |
|---|---|---|---|---|---|---|
| Lipid | ID | ID | p-val | fold change | p-val | fold change |
| AC(13:1) | No ID | No ID | | −1.61 | 7.94E−02 | 1.32 |
| FA(14:3) | No ID | No ID | 5.17E−03 | 1.69 | 4.83E−01 | −1.24 |
| FA(20:3) | No ID | No ID | 6.97E−06 | 18.29 | 1.35E−01 | −2.17 |
| FA(22:4) | No ID | No ID | 7.12E−07 | 34.79 | 3.15E−01 | −1.79 |
| FA(22:5)-1 | No ID | No ID | 2.39E−02 | 3.31 | 1.19E−01 | −1.80 |
| FA(22:5)-2 | No ID | No ID | 2.11E−04 | 3.03 | 1.69E−02 | −1.92 |
| Linoleic acid | C01595 | HMDB00673 | 9.90E−04 | 3.18 | 1.68E−02 | −1.77 |
| Linolenic acid | C06427 | HMDB01388 | 3.57E−05 | 35.88 | 4.08E−02 | −1.99 |
| Oleic acid | C00712 | HMDB00207 | 1.95E−04 | 18.05 | 3.67E−02 | −1.88 |
| Palmitoleic acid | C08362 | HMDB03229 | 9.84E−05 | 2.69 | 2.70E−02 | −1.48 |

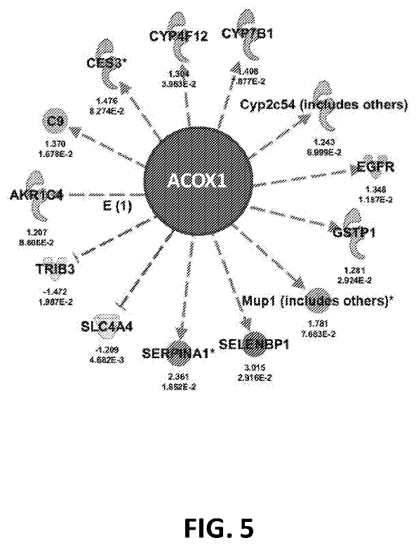

Differential gene expression patterns in the liver impacted by treatment with the amino acid composition were interpreted in the context of the upstream regulator systems biology knowledgebase framework developed by Ingenuity Pathway Analysis. Computed z-scores indicated that the gene expression patterns are consistent with activation of ACOX1, which encodes peroxisomal fatty acid oxidation, as an upstream regulator (FIG. 5 and Table 54).

TABLE 54
P-values and fold changes for gene expression associated with the ACOX1 pathway following
treatment of STAM mice with the amino acid composition (treated) compared to control.

| | | | | Control | Control | Treated | Treated |
|---|---|---|---|---|---|---|---|
| gene | IPA_upstream_regulator | ACOX1_path | IPA_gene_name | fold change | p-val | fold change | p-val |
| Akr1c6 | ACOX1 | ACOX1 | AKR1C4 | −1.68 | 1.88E−06 | 1.207 | 8.606E−02 |
| C9 | ACOX1 | ACOX1 | | −3.10 | 7.81E−07 | 1.370 | 1.678E−02 |
| Ces3a | ACOX1 | ACOX1 | | −2.10 | 2.69E−06 | 1.379 | 3.900E−02 |
| Ces3b | ACOX1 | ACOX1 | | −3.16 | 1.05E−07 | 1.476 | 8.274E−02 |
| Cyp2c50 | ACOX1 | ACOX1 | Cyp2c54 | −1.72 | 1.24E−04 | 1.243 | 6.999E−02 |
| Cyp4a12a | ACOX1 | ACOX1 | | −1.59 | 4.60E−03 | 1.293 | 8.589E−02 |
| Cyp7b1 | ACOX1 | ACOX1 | | −4.45 | 4.29E−04 | 1.408 | 7.877E−02 |
| Egfr | NFKB; ACOX1 | ACOX1 | | −1.98 | 1.31E−04 | 1.348 | 1.187E−02 |
| Gstp1 | ACOX1 | ACOX1 | | −2.31 | 2.56E−06 | 1.281 | 2.924E−02 |
| Mup1 | ACOX1 | ACOX1 | | −7.69 | 1.47E−03 | 1.781 | 7.683E−02 |
| Mup11 | ACOX1 | ACOX1 | | −2.47 | 9.01E−03 | 1.703 | 5.779E−02 |
| Mup14 | ACOX1 | ACOX1 | | −2.05 | 1.27E−02 | 1.395 | 4.890E−02 |
| Mup16 | ACOX1 | ACOX1 | | −6.27 | 4.38E−03 | 1.465 | 7.558E−02 |
| Mup6 | ACOX1 | ACOX1 | | −1.73 | 2.27E−02 | 1.330 | 5.784E−02 |
| Selenbp2 | TGFB; IL10; | ACOX1 | Selenbp1 | −15.77 | 3.73E−05 | 3.015 | 2.916E−02 |
| | ACOX1 | | | | | | |
| Serpina1c | TGFB; ACOX1 | ACOX1 | | −2.25 | 7.22E−09 | 1.290 | 5.612E−02 |
| Serpina1e | TGFB; ACOX1 | ACOX1 | | −43.20 | 3.93E−08 | 2.361 | 1.852E−02 |
| Slc4a4 | ACOX1 | ACOX1 | | 1.55 | 7.00E−06 | −1.209 | 4.682E−03 |
| Trib3 | IL2; NFKB; | ACOX1 | | 2.40 | 3.14E−04 | −1.472 | 1.987E−02 |
| | ACOX1 | | | | | | |

The Amino Acid Composition Tempers Inflammation Pathways

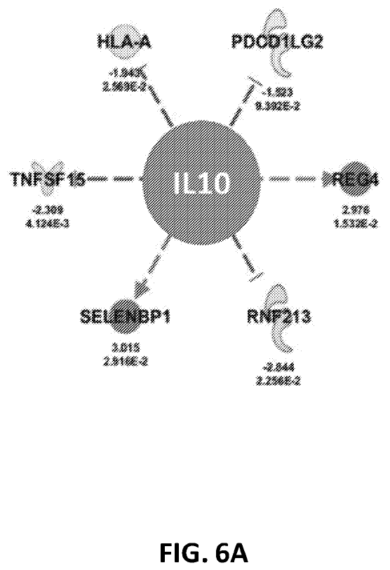

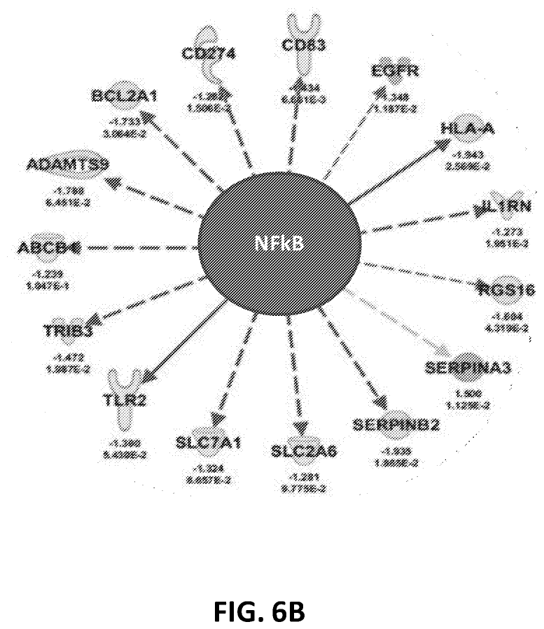

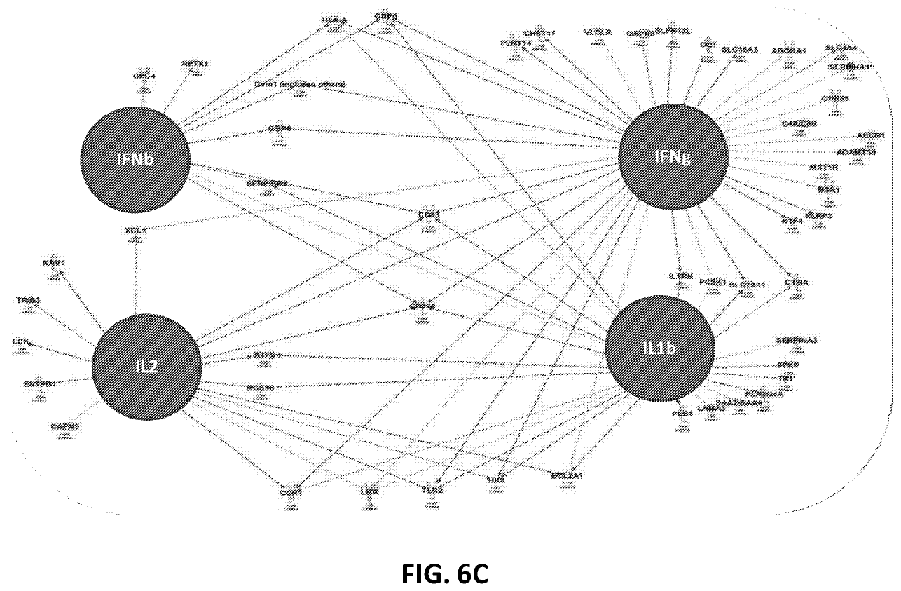

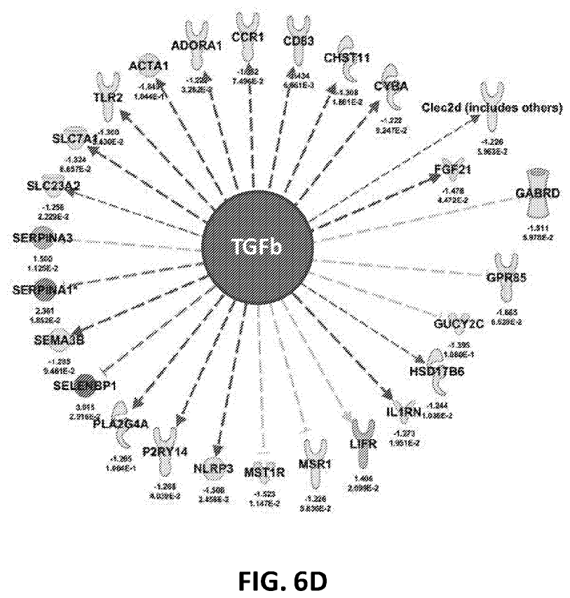

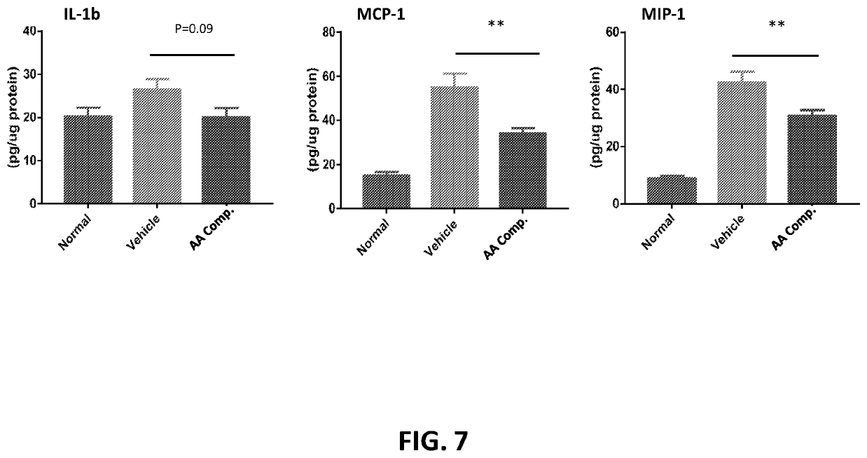

Inflammation is a “second-hit” of NASH. The differential gene expression patterns in the liver as a result of treatment with the amino acid composition yielded z-scores within IPA analysis associated with upstream regulator activation of anti-inflammatory IL-10 (FIG. 6A) and inhibition of pro-inflammatory NF-kB (FIG. 6B and Table 55), interferons, IL-1b, and IL-2 (FIG. 6C and Table 55). At the protein level, treatment with the amino acid composition significantly down-regulated hepatic MCP-1 and MIP-1, which are the ligands of C—C chemokine receptor types 2 (CCR2) and 5 (CCR5), respectively (FIG. 7). Thus, treatment with the amino acid composition tempered the immune system toward an anti-inflammatory state, which may dampen NASH progression.

TABLE 55
P-values and fold changes for gene expression associated with the ACOX1 pathway following
treatment of STAM mice with the amino acid composition (treated) compared to control.

| | IPA | | | | | | | Control | | Treated | |
|---|---|---|---|---|---|---|---|---|---|---|---|
| | upstream | IPA gene | | | IL2 | | | fold | Control | fold | Treated |
| gene | regulator | name | IL10_path | IL1b_path | path | NFKB_path | TGFB_path | change | p-val | change | p-val |
| Abcb1a | NFKB; | | IL10 | | | NFKB | | 3.02 | 1.55E−06 | −1.239 | 1.047E−01 |
| | IL10 | | | | | | | | | | |
| Abcb1b | NFKB; | | IL10 | | | NFKB | | −2.24 | 4.90E−03 | 1.272 | 9.745E−02 |
| | IL10 | | | | | | | | | | |
| Acta1 | TGFB | | | | | | TGFB | 7.96 | 5.87E−03 | −1.849 | 1.044E−01 |
| Adora1 | TGFB | | | | | | TGFB | 1.94 | 1.99E−05 | −1.222 | 3.262E−02 |
| AK007436 | NFKB | ADAMTS9 | | | | NFKB | | 2.32 | 6.38E−03 | −1.788 | 6.451E−02 |
| AK043676 | IL1b | PFKP | | IL1b | | | | 1.68 | 3.12E−03 | −1.462 | 1.586E−02 |
| AK154184 | IL1b; | CYBA | | IL1b | | | TGFB | 1.99 | 1.02E−03 | −1.222 | 9.247E−02 |
| | TGFB | | | | | | | | | | |
| AK158038 | IL2 | NAV1 | | | IL2 | | | 1.62 | 2.63E−02 | −1.659 | 1.587E−02 |
| Atf5 | IL1b; | | | IL1b | IL2 | | | 1.71 | 3.00E−04 | −1.223 | 9.622E−02 |
| | IL2 | | | | | | | | | | |
| Bcl2a1d | IL1b; | | | IL1b | IL2 | NFKB | | 4.00 | 1.74E−04 | −1.733 | 3.064E−02 |
| | IL2; | | | | | | | | | | |
| | NFKB | | | | | | | | | | |
| Capn5 | IL2 | | | | IL2 | | | 1.51 | 2.05E−05 | −1.235 | 5.908E−03 |
| Ccr1 | IL1b; | | | IL1b | IL2 | | TGFB | 2.82 | 1.28E−03 | −1.352 | 7.496E−02 |
| | IL2; | | | | | | | | | | |
| | TGFB | | | | | | | | | | |
| Cd274 | IL1b; | | | IL1b | IL2 | NFKB | | 2.37 | 1.71E−07 | −1.282 | 1.506E−02 |
| | IL2; | | | | | | | | | | |
| | NFKB | | | | | | | | | | |
| Cd83 | IL1b; | | | IL1b | IL2 | NFKB | TGFB | 2.41 | 5.36E−07 | −1.434 | 6.661E−03 |
| | IL2; | | | | | | | | | | |
| | NFKB; | | | | | | | | | | |
| | TGFB | | | | | | | | | | |
| Chst11 | TGFB | | | | | | TGFB | 2.91 | 7.87E−06 | −1.308 | 1.881E−02 |
| Clec2i | TGFB | | | | | | TGFB | 1.80 | 1.01E−03 | −1.226 | 5.963E−02 |
| Egfr | NFKB | | | | | NFKB | | −1.98 | 1.31E−04 | 1.348 | 1.187E−02 |
| Entpd1 | IL2 | | | | IL2 | | | 1.87 | 1.84E−04 | −1.218 | 8.413E−02 |
| Fgf21 | TGFB | | | | | | TGFB | 49.56 | 1.03E−03 | −1.478 | 4.472E−02 |
| Gabrd | TGFB | | | | | | TGFB | 5.82 | 1.48E−04 | −1.511 | 5.978E−02 |
| Gbp4 | IL1b; | Gbp6 | IL10 | IL1b | | | | 1.60 | 1.01E−03 | −1.234 | 5.234E−02 |
| | IL10 | | | | | | | | | | |
| Gbp5 | IL10 | | IL10 | | | | | 1.81 | 7.82E−05 | −1.217 | 6.120E−02 |
| Gm8909 | IL1b; | HLA-A | IL10 | IL1b | | NFKB | | 4.03 | 8.19E−04 | −1.943 | 2.569E−02 |
| | NFKB; | | | | | | | | | | |
| | IL10 | | | | | | | | | | |
| Gpr85 | TGFB | | | | | | TGFB | 2.46 | 1.77E−02 | −1.685 | 6.628E−02 |
| Gucy2c | TGFB | | | | | | TGFB | 2.35 | 8.97E−03 | −1.395 | 1.080E−01 |
| Hk2 | IL1b; | | | IL1b | IL2 | | | 2.00 | 2.01E−04 | −1.301 | 9.834E−02 |
| | IL2 | | | | | | | | | | |
| Hsd17b6 | TGFB | | | | | | TGFB | 2.89 | 4.20E−05 | −1.244 | 1.036E−02 |
| Il1rn | IL1b; | | IL10 | IL1b | | NFKB | TGFB | 4.43 | 5.94E−09 | −1.273 | 1.951E−02 |
| | NFKB; | | | | | | | | | | |
| | TGFB; | | | | | | | | | | |
| | IL10 | | | | | | | | | | |
| Lama3 | IL1b | | | IL1b | | | | −3.09 | 1.90E−05 | 1.650 | 5.542E−02 |
| Lck | IL2 | | | | IL2 | | | 1.92 | 8.87E−04 | −1.246 | 3.027E−02 |
| Lifr | IL1b; | | | IL1b | IL2 | | TGFB | −4.43 | 2.43E−05 | 1.406 | 2.099E−02 |
| | IL2; | | | | | | | | | | |
| | TGFB | | | | | | | | | | |
| Msr1 | TGFB | | | | | | TGFB | 1.58 | 1.13E−03 | −1.226 | 5.836E−02 |
| Mst1r | TGFB | | | | | | TGFB | 2.08 | 4.17E−03 | −1.523 | 1.147E−02 |
| Nlrp3 | TGFB | | | | | | TGFB | 1.93 | 1.30E−03 | −1.506 | 2.456E−02 |
| P2ry14 | TGFB | | | | | | TGFB | 3.29 | 4.67E−05 | −1.268 | 4.039E−02 |
| Pcsk1 | IL1b | | | IL1b | | | | 2.07 | 9.00E−03 | −2.070 | 8.150E−02 |
| Pla2g4a | IL1b; | | | IL1b | | | TGFB | 2.10 | 7.94E−05 | −1.265 | 1.064E−01 |
| | TGFB | | | | | | | | | | |
| Plb1 | IL1b | | | IL1b | | | | 2.01 | 2.09E−02 | −1.696 | 2.324E−02 |
| Rgs16 | IL1b; | | | IL1b | IL2 | NFKB | | 7.68 | 2.47E−05 | −1.604 | 4.319E−02 |
| | IL2; | | | | | | | | | | |
| | NFKB | | | | | | | | | | |
| Saa4 | IL1b | | | IL1b | | | | −1.72 | 4.76E−02 | 1.283 | 9.771E−02 |
| Selenbp2 | TGFB; | Selenbp1 | IL10 | | | | TGFB | −15.77 | 3.73E−05 | 3.015 | 2.916E−02 |
| | IL10 | | | | | | | | | | |
| Sema3b | TGFB | | | | | | TGFB | 4.12 | 4.78E−05 | −1.285 | 9.461E−02 |
| Serpina1c | TGFB | | | | | | TGFB | −2.25 | 7.22E−09 | 1.290 | 5.612E−02 |
| Serpina1e | TGFB | | | | | | TGFB | −43.20 | 3.93E−08 | 2.361 | 1.852E−02 |
| Serpina3k | IL1b; | | | IL1b | | NFKB | TGFB | −2.95 | 1.85E−08 | 1.500 | 1.125E−02 |
| | NFKB; | | | | | | | | | | |
| | TGFB | | | | | | | | | | |
| Serpinb2 | IL1b; | | | IL1b | | NFKB | | 1.94 | 4.98E−02 | −1.935 | 1.865E−02 |
| | NFKB | | | | | | | | | | |
| Slc23a2 | TGFB | | | | | | TGFB | 2.00 | 1.02E−05 | −1.258 | 2.229E−02 |
| Slc2a6 | NFKB; | | IL10 | | | NFKB | | 1.79 | 2.68E−02 | −1.281 | 9.775E−02 |
| | IL10 | | | | | | | | | | |
| Slc7a1 | NFKB; | | | | | NFKB | TGFB | 1.64 | 3.96E−03 | −1.324 | 8.657E−02 |
| | TGFB | | | | | | | | | | |
| Slc7a11 | IL1b; | | IL10 | IL1b | | | | 65.45 | 1.35E−03 | −1.869 | 1.068E−01 |
| | IL10 | | | | | | | | | | |
| Tk1 | IL1b | | | IL1b | | | | −2.40 | 4.99E−06 | 1.295 | 4.036E−02 |
| Tlr11 | IL10 | | IL10 | | | | | 1.66 | 6.11E−03 | −1.368 | 6.005E−02 |
| Tlr2 | IL1b; | | IL10 | IL1b | IL2 | NFKB | TGFB | 2.12 | 5.32E−05 | −1.300 | 5.430E−02 |
| | IL2; | | | | | | | | | | |
| | NFKB; | | | | | | | | | | |
| | TGFB; | | | | | | | | | | |
| | IL10 | | | | | | | | | | |
| Trib3 | IL2; | | | | IL2 | NFKB | | 2.40 | 3.14E−04 | −1.472 | 1.987E−02 |
| | NFKB | | | | | | | | | | |
| Xcl1 | IL2 | | | | IL2 | | | 2.52 | 1.22E−03 | −1.796 | 6.279E−02 |

The Amino Acid Composition Prevents Fibrogenesis Pathways

Fibrosis is at the nexus of several biologic processes, such as metabolic dysregulation, inflammation, and cell death. Lipid accumulation in hepatocytes and chronic inflammation induce fibrogenic activation of hepatic stellate cells (Wobser H, et al., Cell Res. 2009, which is hereby incorporated by reference in its entirety). The liver gene expression pattern resulting from treatment with the amino acid composition was consistent with the suppression of the fibrogenic TGF-b signaling pathway (FIG. 6D).

Increasing evidence implicates that CCR2/CCR5 and their ligands, including MCP-1/MIP-1, promote macrophage recruitment and hepatic stellate cell activation which contribute to fibrosis following liver tissue damage (Lefebvre E, et al., PLoS One 2016, which is hereby incorporated by reference in its entirety). The amino acid composition displayed a potent antifibrotic activity in the STAM model of NASH via reducing hepatic TGF-b signaling and MCP-1 and MIP-1 proteins (FIG. 7).

### CONCLUSION

The amino acid composition demonstrated consistent disease modifying activity in both STAM and FATZO mouse models of NASH including improvement in NAS and amelioration of ballooning and fibrosis. The activity of the amino acid composition appears to be driven, at least in part, via increase in fatty acid oxidation, reduction in levels of key cytokines and transcription pathways associated with liver inflammation and fibrosis.

### Example 4. Hepatocyte Model for Steatosis and Inflammation

Hepatocyte lipotoxicity appears to be a central driver of hepatic cellular injury via oxidative stress and endoplasmic reticulum (ER) stress. The ability of amino acids to influence steatosis (lipid accumulation) and inflammation in hepatocytes was assessed using human primary hepatocytes (Lonza, TRL).

### Cell Seeding and Maintenance

Primary hepatocytes lot nos. from two healthy human donors were seeded on day 0 at density of 6e04 cells in 96 well optical microplates (Thermofisher) in hepatocyte plating media (William's E medium (Gibco) supplemented with 10% heat-inactivated FBS (Atlanta Bio), 2 mM Glutamax (Gibco), and 0.2% Primocin (InVivoGen) and incubated for 6 hours at 37° C., 5% CO2. After 6 hours, cells were washed twice and incubated overnight at 37° C., 5% CO2 with Hepatocytes defined medium (Corning) supplemented with 2 mM Glutamax (Gibco), and 1× Penicillin/Streptomycin. On day 1, cells were washed twice and incubated for 24 h in the hepatocyte culture media in the same conditions described above.

### Amino Acids Pre-Treatment

On day 2, cells were washed twice with DPBS 1× (Gibco) and maintained in amino acid-free WEM (US Biologicals) containing a defined custom amino acid concentration based on the mean physiological concentrations in blood. The values are published in the Human Metabolome Database (Wishart D S, Tzur D, Knox C, et al., HMDB: the Human Metabolome Database. Nucleic Acids Res. 2007 January; 35(Database issue):D521-6. 17202168; which is hereby incorporated by reference in its entirety). This custom media is supplemented with 11 mM Glucose, 0.272 mM Sodium Pyruvate, and a dose curve of defined amino acid compositions (i.e., vehicle, LIVRQ+N-acetylcysteine, LIVRQ, RQ+N-acetylcysteine, N-acetylcysteine alone, LIV, or individually with L-Leucine, L-Isoleucine, L-Valine, L-Arginine, L-Glutamine, and L-Cysteine) at various ranges of concentrations. Cells were maintained in this defined media for 24 hours at 37° C., 5% CO2.

Co-Treatment with Free Fatty Acids and Different Amino Acids Combination

After pre-treatment, cells were exposed to free fatty acids (FFA) at 250 uM with a ratio of 2:1 (Oleate:Palmitate) supplemented with TNF-α (Thermofisher) at 1 ng/ml or vehicle. Cells were incubated with the FFAs mixture and the different amino acids combinations for 24 hours at 37° C., 5% CO2. After 24 hours incubation, media was removed for cytokine analysis and replaced by fresh media containing the same stimulus conditions and amino acid concentrations. Cells were incubated for an additional 48 hours for a total of 72 hours of FFA and TNFα stimulation.

Cytokine Analysis after 24 h by ELISA

Human CCL2 (MCP-1) was measured by ELISA (Human CCK2/MCP-1 DuoSet ELISA, R&D Systems) at 1/5 or 1/10 dilution in 1× Reagent Diluent (Reagent Ancillary Kit 2, R&D Systems). Data were normalized to the specific per well cell density determined by nuclei count stained by Hoechst 3342 (Life technologies) in the fluorescence microscopy described below.

Intracellular Lipid Accumulation Analysis after 72 h by Fluorescence Microscopy

After 72 hours, cells were washed twice in 100 ul PBS 1× (Gibco), fixed with 4% Paraformaldehyde, and washed twice with PBS 1× (100 ul). After fixation, lipids were stained with HCS LipidTOX Red Neutral (Thermofisher Scientific) diluted 1000× and nuclei were stained with Hoechst 3342 (Life Technologies) diluted to 4 ug/ml. The LipidTOX™ neutral lipid stain has an extremely high affinity for neutral lipid droplets that was detected by fluorescence microscopy using a high content imager (Molecular Devices).

### Results

### Lipid Accumulation and Steatosis Phenotypes

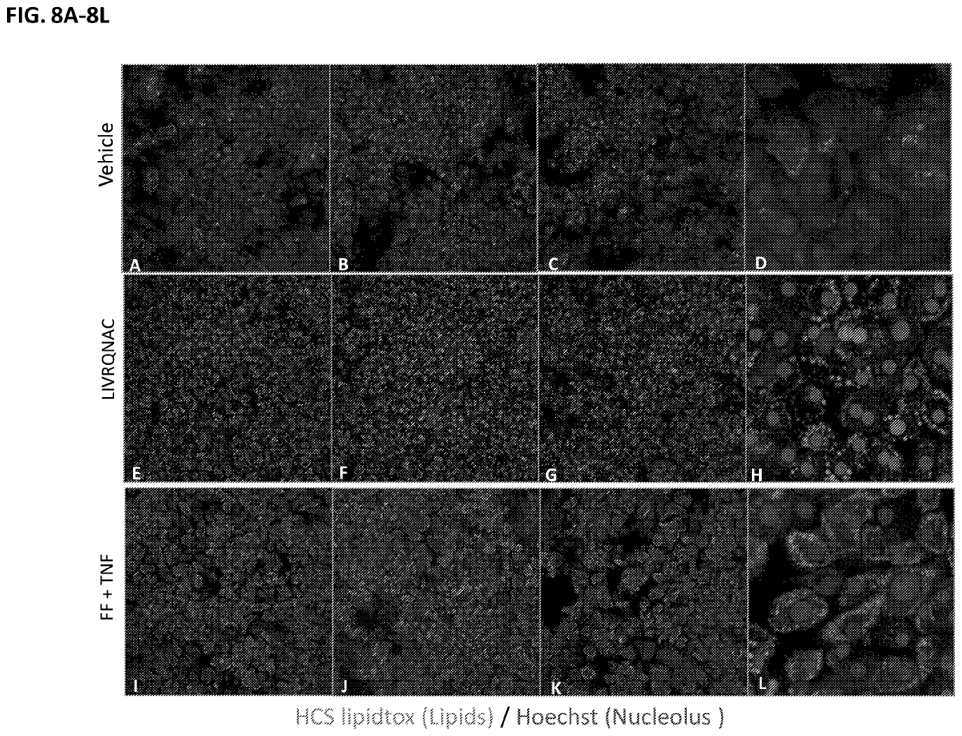

Primary human hepatocytes from healthy donors were found to have low levels of lipid accumulation (FIG. 8A-8D). Treatment of the cells with free fatty acids (FF)+TNFα induced lipid accumulation (FIG. 8I-8L) with a macro-steatosis phenotype. Treatment with LIVRQNAC changed the hepatocyte phenotypes from macro-steatosis to micro-steatosis (FIG. 8E-8H).

### MCP1/CCL2 Secretion

Tables 56-59 show the baseline subtracted secretion of MCP1/CCL2 in primary human hepatocytes cells from two healthy donors (donor 1 for Tables 56 and 57, and donor 2 for Tables 58 and 59). LIVRQNAC, LIVRQNAC+G, LIVRQNAC+S, LIVRQ and RQNAC significantly decreased MCP1/CCL2 secretion in both donors. The combination LIV, however, significantly increased MCP1/CCL2 secretion only in one of the donors. The addition of arginine (R) and glutamine (Q) to a combination of LIV decreased the secretion of MCP1/CCL2 in both donors compared to LIV alone. Individually, N-acetyl cysteine and glutamine are shown to significantly decrease MCP1/CCL2 secretion, while arginine increased MCP1 secretion. Isoleucine, Leucine and Valine did not have an effect on MCP1/CCL2 secretion.

TABLE 56
Changes in MCP1 expression for donor 1 upon
administration of amino acid compositions

| | | MCP1 expression relative to Control - Donor 1 | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −24.1616 | 0.032252 | 3 | 0.0001 | **** |
| LIVRQNAC | 30 | −22.2916 | 2.119583 | 3 | 0.0001 | **** |
| LIVRQNAC | 20 | −18.4363 | 0.850597 | 3 | 0.0005 | *** |
| LIVRQNAC | 10 | −14.3383 | 1.854977 | 3 | 0.0074 | ** |
| LIVRQNAC | 1 | 0 | 1.048045 | 3 | | |
| LIVRQNAC + G | 40 | −22.0824 | 0.873105 | 3 | 0.0001 | **** |
| LIVRQNAC + G | 30 | −19.2605 | 1.611788 | 3 | 0.0003 | *** |
| LIVRQNAC + G | 20 | −17.5807 | 2.893835 | 3 | 0.0009 | *** |
| LIVRQNAC + G | 10 | −13.7521 | 3.068991 | 3 | 0.0106 | * |
| LIVRQNAC + G | 1 | 0 | 1.682719 | 3 | | |
| LIVRQNAC + S | 40 | −32.4703 | 0.340537 | 3 | 0.0001 | **** |
| LIVRQNAC + S | 30 | −30.768 | 1.339048 | 3 | 0.0001 | **** |
| LIVRQNAC + S | 20 | −25.5964 | 1.854519 | 3 | 0.0001 | **** |
| LIVRQNAC + S | 10 | −17.8326 | 1.974033 | 3 | 0.0008 | *** |
| LIVRQNAC + S | 1 | 2.37E−15 | 18.41384 | 3 | | |
| LIV | 40 | 15.52052 | 6.323205 | 3 | 0.0094 | ** |
| LIV | 30 | 12.3111 | 10.02706 | 3 | 0.0475 | * |
| LIV | 20 | 12.6686 | 4.109608 | 3 | 0.0401 | * |
| LIV | 10 | −5.18869 | 1.579468 | 3 | 0.6477 | ns |
| LIV | 1 | −1.2E−15 | 8.178943 | 3 | | |
| LIVRQ | 40 | −25.9576 | 0.484283 | 3 | 0.0028 | ** |
| LIVRQ | 30 | −23.6562 | 2.599721 | 3 | 0.0099 | ** |
| LIVRQ | 20 | −13.4723 | 3.427666 | 3 | 0.6401 | ns |
| LIVRQ | 10 | −9.22141 | 7.599407 | 3 | 0.9986 | ns |
| LIVRQ | 1 | −8.23198 | 5.80889 | 3 | | |
| RQNAC | 40 | −21.4681 | 2.903892 | 3 | 0.0003 | *** |
| RQNAC | 30 | −17.1873 | 5.202568 | 3 | 0.0038 | ** |
| RQNAC | 20 | −12.1782 | 2.907484 | 3 | 0.0506 | ns |
| RQNAC | 10 | −8.89378 | 4.748653 | 3 | 0.206 | ns |
| RQNAC | 1 | 1.18E−15 | 10.02527 | 3 | | |
| N-Acetyl | 40 | −17.6065 | 1.211739 | 3 | 0.0009 | *** |
| Cysteine | | | | | | |
| N-Acetyl | 20 | −10.8919 | 2.27818 | 3 | 0.0545 | ns |
| Cysteine | | | | | | |
| N-Acetyl | 10 | −2.49755 | 8.795693 | 3 | 0.9424 | ns |
| Cysteine | | | | | | |
| N-Acetyl | 5 | −0.76286 | 7.457085 | 3 | 0.9991 | ns |
| Cysteine | | | | | | |
| N-Acetyl | 0 | 0 | 6.716428 | 3 | | |
| Cysteine | | | | | | |

TABLE 57
Changes in MCP1 expression for donor 1 upon administration
of single amino acid compositions

| | | MCP1 expression relative to Control- Donor 1 | | | | |
|---|---|---|---|---|---|---|
| Amino Acid | Conc. | | Std. | Number | | |
| Supplement | (μM) | Mean | Deviation | of values | P-value* | Significance |
| Valine | 23420 | 14.16805 | 19.23365 | 3 | 0.6777 | ns |
| Valine | 11710 | 77.73396 | 137.82 | 3 | 0.9998 | ns |
| Valine | 4684 | 23.6867 | 46.48697 | 3 | 0.2502 | ns |
| Valine | 234 | −2.4E−15 | 13.86902 | 3 | | |
| Arginine | 5440 | 10.9386 | 4.79774 | 3 | 0.0057 | ** |
| Arginine | 2720 | 6.526801 | 4.266971 | 3 | 0.1517 | ns |
| Arginine | 1088 | 5.114414 | 4.685563 | 3 | 0.3321 | ns |
| Arginine | 109 | 2.37E−15 | 0.666016 | 3 | | |
| Glutamine | 22484 | −21.8392 | 1.113443 | 3 | 0.0004 | *** |
| Glutamine | 11242 | −9.00139 | 1.68951 | 3 | 0.2459 | ns |
| Glutamine | 3747 | −0.89805 | 6.374471 | 3 | 0.9991 | ns |
| Glutamine | 749 | 0 | 9.549143 | 3 | | |
| Isoleucine | 6639 | −0.205 | 2.292188 | 3 | 0.9998 | ns |
| Isoleucine | 3320 | −2.41722 | 2.382379 | 3 | 0.4907 | ns |
| Isoleucine | 1328 | −0.30729 | 2.409691 | 3 | 0.9992 | ns |
| Isoleucine | 66 | −1.2E−15 | 3.163838 | 3 | | |
| Leucine | 15270 | −1.36762 | 3.37035 | 3 | 0.8675 | ns |
| Leucine | 7635 | 1.895506 | 3.757642 | 3 | 0.6872 | ns |
| Leucine | 3054 | 3.340489 | 3.016641 | 3 | 0.2201 | ns |
| Leucine | 153 | 5.92E−16 | 3.132507 | 3 | | |
| N-Acetyl | 10000 | −17.6065 | 1.211739 | 3 | 0.0009 | *** |
| Cysteine | | | | | | |
| N-Acetyl | 5000 | −10.8919 | 2.27818 | 3 | 0.0545 | ns |
| Cysteine | | | | | | |
| N-Acetyl | 2500 | −2.49755 | 8.795693 | 3 | 0.9424 | ns |
| Cysteine | | | | | | |
| N-Acetyl | 1000 | −0.76286 | 7.457085 | 3 | 0.9991 | ns |
| Cysteine | | | | | | |
| N-Acetyl | 0 | 0 | 6.716428 | 3 | | |
| Cysteine | | | | | | |

TABLE 58
Changes in MCP1 expression for donor 2 upon
administration of amino acid compositions

| | | MCP1 expression relative to Control - Donor 2 | | | | |
|---|---|---|---|---|---|---|
| Amino Acid | Conc. | | Std. | Number | | |
| Supplement | (X) | Mean | Deviation | of values | P-value* | Significance |
| LIVRQNAC | 40 | −24.5376 | 1.632923 | 3 | 0.0001 | **** |
| LIVRQNAC | 30 | −13.6824 | 2.562571 | 3 | 0.0001 | **** |
| LIVRQNAC | 20 | −8.42053 | 1.545343 | 3 | 0.0001 | **** |
| LIVRQNAC | 10 | 2.126223 | 0.453924 | 3 | 0.0007 | *** |
| LIVRQNAC | 1 | −4.7E−15 | 0.412226 | 3 | | |
| LIVRQNAC + G | 40 | −35.3651 | 2.08381 | 3 | 0.0007 | *** |
| LIVRQNAC + G | 30 | −30.3247 | 5.225183 | 3 | 0.001 | *** |
| LIVRQNAC + G | 20 | −17.0719 | 4.522244 | 3 | 0.0119 | * |
| LIVRQNAC + G | 10 | −14.2586 | 2.767898 | 3 | 0.049 | * |
| LIVRQNAC + G | 1 | −7.1E−15 | 7.613666 | 3 | | |
| LIVRQNAC + S | 40 | −35.8381 | 1.404782 | 3 | 0.0001 | **** |
| LIVRQNAC + S | 30 | −30.9946 | 2.372062 | 3 | 0.0001 | **** |
| LIVRQNAC + S | 20 | −16.8831 | 3.223007 | 3 | 0.0004 | *** |
| LIVRQNAC + S | 10 | −5.60595 | 10.2119 | 3 | 0.1887 | |
| LIVRQNAC + S | 1 | 2.37E−15 | 4.4168 | 3 | | |
| LIV | 40 | −46.7898 | 8.664441 | 3 | 0.3692 | ns |
| LIV | 30 | −34.5953 | 16.84743 | 3 | 0.6246 | ns |
| LIV | 20 | −28.0851 | 31.84348 | 3 | 0.7684 | ns |
| LIV | 10 | −11.0006 | 72.74556 | 3 | 0.9889 | ns |
| LIV | 1 | 9.47E−15 | 60.93638 | 3 | | |
| LIVRQ | 40 | −129.802 | 7.067989 | 3 | 0.0008 | *** |
| LIVRQ | 30 | −110.034 | 4.53852 | 3 | 0.0042 | ** |
| LIVRQ | 20 | −33.3611 | 31.87706 | 3 | 0.6524 | |
| LIVRQ | 10 | −3.30904 | 71.03267 | 3 | 0.9999 | |
| LIVRQ | 1 | −4.7E−15 | 46.12987 | 3 | | |
| RQNAC | 40 | −133.48 | 1.908424 | 3 | 0.0006 | *** |
| RQNAC | 30 | −123.712 | 1.043889 | 3 | 0.0013 | ** |
| RQNAC | 20 | −109.575 | 5.533323 | 3 | 0.0044 | ** |
| RQNAC | 10 | −55.8583 | 22.72309 | 3 | 0.2273 | |
| RQNAC | 1 | 1.42E−14 | 43.79031 | 3 | | |
| N-Acetyl | 10000 | −28.4419 | 1.694 | 3 | 0.0001 | *** |
| Cysteine | | | | | | |
| N-Acetyl | 5000 | −10.5725 | 4.362178 | 3 | 0.0012 | ** |
| Cysteine | | | | | | |
| N-Acetyl | 2500 | −4.0591 | 5.600773 | 3 | 0.0572 | ns |
| Cysteine | | | | | | |
| N-Acetyl | 1000 | 1.602474 | 3.423109 | 3 | 0.0001 | **** |
| Cysteine | | | | | | |
| N-Acetyl | 0 | 0 | 2.068861 | 3 | | |
| Cysteine | | | | | | |

TABLE 59
Changes in MCP1 expression for donor 2 upon administration
of single amino acid compositions

| | | MCP1 expression relative to Control- Donor 2 | | | | |
|---|---|---|---|---|---|---|
| Amino Acid | Conc. | | Std. | Number | | |
| Supplement | (μM) | Mean | Deviation | of values | P-value* | Significance |
| Valine | 23420 | −30.7921 | 22.55378 | 3 | 0.6118 | ns |
| Valine | 11710 | 38.24762 | 28.44112 | 3 | 0.4268 | ns |
| Valine | 4684 | 10.79011 | 51.87642 | 3 | 0.9835 | ns |
| Valine | 234 | −1.4E−14 | 30.91388 | 3 | | |
| Arginine | 5440 | 8.493664 | 22.98385 | 3 | 0.9913 | ns |
| Arginine | 2720 | 24.06261 | 63.49489 | 3 | 0.7429 | ns |
| Arginine | 1088 | 24.95224 | 52.94171 | 3 | 0.7192 | ns |
| Arginine | 109 | −4.7E−15 | 11.27976 | 3 | | |
| Glutamine | 22484 | −138.873 | 10.74317 | 3 | 0.0001 | **** |
| Glutamine | 11242 | −90.6558 | 15.43989 | 3 | 0.0037 | ** |
| Glutamine | 3747 | −45.0574 | 41.63249 | 3 | 0.2474 | ns |
| Glutamine | 749 | 2.84E−14 | 59.86955 | 3 | 0.7631 | |
| Isoleucine | 6639 | 18.62132 | 26.01824 | 3 | 0.5663 | ns |
| Isoleucine | 3320 | −5.64461 | 7.719105 | 3 | 0.9882 | ns |
| Isoleucine | 1328 | 26.62309 | 5.65413 | 3 | 0.2613 | ns |
| Isoleucine | 66 | 0 | 4.245462 | 3 | | |
| Leucine | 15270 | −26.6436 | 10.08177 | 3 | 0.2607 | ns |
| Leucine | 7635 | −2.98815 | 21.00205 | 3 | 0.9989 | ns |
| Leucine | 3054 | 16.11014 | 8.662188 | 3 | 0.68 | ns |
| Leucine | 153 | −4.7E−15 | 7.63396 | 3 | | |
| N-Acetyl | 10000 | −28.4419 | 1.694 | 3 | 0.0001 | *** |
| Cysteine | | | | | | |
| N-Acetyl | 5000 | −10.5725 | 4.362178 | 3 | 0.0012 | ** |
| Cysteine | | | | | | |
| N-Acetyl | 2500 | −4.0591 | 5.600773 | 3 | 0.0572 | ns |
| Cysteine | | | | | | |
| N-Acetyl | 1000 | 1.602474 | 3.423109 | 3 | 0.0001 | **** |
| Cysteine | | | | | | |
| N-Acetyl | 0 | 0 | 2.068861 | 3 | | |
| Cysteine | | | | | | |

### Example 5. Treatment of NASH in a Mouse Model with an Amino Acid Composition Induction of NASH in Mice

In one example, the effects of LIVRQNAC and related amino acid compositions in the obesity, metabolism-driven non-alcoholic steatohepatitis (NASH) in FATZO mouse model was examined.

### Induction of NASH in Mice

NASH was induced in 60 male FATZO mice by a western diet (Research Diet #D12079B; fat 40% kcal, protein 17% kcal, carbohydrate 43% kcal) supplemented with 5% fructose in the drinking water (WDF) during a 16 week induction phase. Diets and water were available ad libitum. Littermate control male FATZO mice fed with a control diet (n=6, Purina #5008; fat 17% kcal, protein 27% kcal, carbohydrate 56% kcal) and sterile water were set up for control purpose. Mice were housed in plastic cages with microisolator. Sterilized bedding was replaced once a week. Mice were housed three per cage and maintained on a twelve hour light cycle throughout study duration. Room temperature was monitored daily and maintained at 22−25° C. Body weight was recorded every week during the induction phase.

### Study Design

Following 16 weeks diet induction, 6 mice remained on control diet (group 1, Control) while 60 induced mice were randomized on body weight and plasma glucose (fed) for assignment to the following treatments. FATZO mice were administered with test articles starting at 16 weeks post western diet NASH induction for 4 weeks. Test articles were administered by oral gavage. Animals were euthanized at 20 weeks post western diet NASH induction, and tissues were harvested for analysis.

| Group | (n) | Treatment (oral) | Diet |
|---|---|---|---|
| 1 | 6 | Vehicle | 5008 WDF |
| 2 | 10 | Vehicle | D12079B + 5% Fructose |
| 3 | 10 | LIVRQNAC (1500 mg/kg) | D12079B + 5% Fructose |
| 4 | 10 | LIVRQNAC (3000 mg/kg) | D12079B + 5% Fructose |
| 5 | 10 | LIVRQNAC + G (3885 mg/kg) | D12079B + 5% Fructose |
| 6 | 10 | LRQNAC (2469 mg/kg) | D12079B + 5% Fructose |
| 7 | 10 | Obeticholic acid (OCA) | D12079B + 5% Fructose |
| | | 30 mg/kg/day | |

### Test Articles

LIVRQNAC, LIVRQNAC+G, LRQNAC, and OCA (Advanced ChemBlocks, Inc.), incipient, and water for irrigation were provided by Axcella Health, Inc. 0.5% Methylcellulosewas provided by CrownBio, Inc. Dosing solutions were prepared according to Appendix 1. TA compounds (amino acid compositions) were amino acid blends formulated fresh daily in water for irrigation (Baxter #27F7114) and the excipients 0.125% Xanthan Gum, 1.5 mM Sodium Lauryl Sulfate and 0.28% Lecithin. Obeticholic acid (OCA) was suspended in 0.5% methylcellulose in water for irrigation. All test articles were stored refrigerated. TA compounds were provided in frozen powder form by the sponsor. Dosing was continued for 4 weeks.

Leucine dosages of LIVRQNAC+G and LRQNAC were matched to that of LIVRQNAC.

| | | | Supplier | |
|---|---|---|---|---|
| | | | Part | Lot |
| Ingredient | Grade | Supplier | Number | Number |
| Fusi-BCAA | Instantized | Ajinomoto | 33555 | OH704 |
| Unflavored (2:1:1 | (0.3-0.9% | (AjiPure) | | |
| L-Leu:L-Ile:L- | Lecithin) | | | |
| Val) | | | | |
| L-Arginine HCl | USP | Sigma | A4599 | CDB0352V |
| | | (Ajinomoto) | | |
| L-Arginine HCl | USP | Sigma | A4599 | CDB0352V |
| | | (Ajinomoto) | | |
| L-Glutamine | USP | Ajinomoto | 32824 | R014A003 |
| Glycine | USP | Ajinomoto | 30359 | R015T008 |
| Acetylcysteine | USP | Spectrum | AC126 | 1FI0576 |
| (NAC) | | Chemical | | |

| | LIVRQNAC | LIVRQNAC + G | LRQNAC |
|---|---|---|---|
| | Daily | Daily | Daily |
| Ingredient | Dose (g) | Dose (g) | Dose (g) |
| Fusi-BCAA Unflavored | 24.0 | 24.0 | |
| (2:1:1 L-Leu:L-Ile:L-Val) | | | |
| Fusil (L-Leucine) | | | 12.0 |
| L-Arginine HCl | 18.0 | 18.0 | 18.0 |
| L-Glutamine | 24.0 | 24.0 | 24.0 |
| Glycine | | 20.0 | |
| Acetylcysteine (NAC) | 1.8 | 1.8 | 1.8 |
| AMINO ACIDS= | 67.8 | 87.8 | 55.8 |

LIVRQNAC, LIVRQNAC+G, LRQNAC, OCA and Vehicle were administered by oral gavage at a volume of 10 mL/kg throughout the study. Dosages were calculated by daily body weight. LIVRQNAC, LIVRQNAC+G, LRQNAC, and Vehicle were administered twice per day (BID), while OCA was administered once a day (QD) in the morning. Mice receiving OCA once per day (QD), and one vehicle QD. Doses were administered by oral gavage at 0700 and 1800 by oral gavage for 4 weeks.

### Body Weight and Blood Glucose

The viability, clinical signs and behavior were monitored daily. Body weight was recorded daily during the dosing period. Blood samples were collected weekly in the AM (0700) via tail clip for glucose measurement (StatStrip glucometer).

### Necropsy and Sample Harvest

Animals were anesthetized with CO2 inhalation and exsanguinated via cardiac puncture for euthanasia. Terminal blood samples (K2EDTA) were obtained by cardiac puncture in anesthetized animals at termination. Samples were provided frozen to Axcella Health. Organ weights (total liver, gonadal fat pads) were recorded. Pancreas, and small intestine and gonadal fat pads were fixed in 10% Buffered Formalin and prepared as directed in protocol. A section of small intestine, gonadal fat pad and liver were also snap frozen in liquid nitrogen and shipped to the sponsor.

### Histological Analyses

The liver tissues were fixed in Bouin's solution at 4° C. for 24 hours followed by baths of standard concentrations of alcohol then xylene to prepare the tissues for paraffin embedding. After being embedded in paraffin and cooled, five-micron sections were cut and stained for routine H&E and Picric Sirius Red. A section of both right and left lobes of the livers were frozen in OCT for analysis of lipid content with Oil-Red-) staining. The Aperio whole slide digital imaging system (Scan Scope C S, Vista, Calif.) was used for imaging. All slides were imaged at 20×. The scan time ranged from 1.5 minutes to a maximum time of 2.25 minutes. The whole images were housed and stored in their Spectrum software system and images were shot from the whole slides.

The livers were evaluated using the NASH liver criteria for scoring. In this mouse study, one cross section of liver for each case was analyzed with the NASH score system. According to the published NASH CRN Scoring System, this scoring system comprises of NAFLD Activity Score (NAS), fibrosis stage and identification of NASH by pattern recognition. The NAS can range from 0 to 8 and is calculated by the sum of scores of steatosis (0-3), lobular inflammation (0-3) and hepatocyte ballooning (0-2) from H&E stained sections. Fibrosis was scored (0-4) from picrosirius red stained slides. The NASH system is used for human liver 18 gauge biopsies. Steatosis, lobular inflammation, hepatocyte. balloon degeneration, fibrosis, NAS and the presence of NASH by pattern recognition were systematically assessed. In this study we evaluated one total cross section of liver per mouse in this study. This is about 15 times the size of an 18 gauge human liver biopsy. The pathology score was determined as 0, +1, +2, or +3. The lesions were scored on location (periportal, centrilobular, and mid zonal) and fat accumulation (focal, periportal, and/or centrilobular). The other part of the score was distribution of the lesions: focal, multifocal and/or diffuse. Also, mild, moderate and severity of the lesions. These parameters made up the total NASH score.

All immunohistochemical staining steps were performed using the Dako FLEX SYSTEM on an automated immunostainer; incubations were done at room temperature and Tris buffered saline plus 0.05% Tween 20, pH 7.4 (TBS—Dako Corp.) was used for all washes and diluents. Thorough washing was performed after each incubation. Primary antibodies included anti-mouse SMA, F4/80, Mac-2, and Picric Sirius Red. Control sections were treated with an isotype control using the same concentration as primary antibodies to verify the staining specificity.

White adipose tissue (WAT) adipocyte size was analyzed from the H&E stained sections. Using the Aperio Image Scope application, 3 localized regions (edge of tissue, tissue not surrounding vascular area, tissue surrounding vascular area) of each tissue specimen were assessed by measuring the area of 10 largest adipocytes of the region. Within each tissue, 10 hot spots of each regions were quantified (um²) and averaged.

Pancreatic beta-islet cells were identified by immunohistochemical staining.

### Image Analysis

Aperio Automatic Image Quantitation was employed to quantify positive pixels of immunohistochemical staining, Oil-Red 0, and Sirius Red staining. The Positive Pixel Count algorithm was used to quantify the percentage of a specific stain present in a scanned slide image. A range of color (range of hues and saturation) and three intensity ranges (weak, positive, and strong) were masked and evaluated. The algorithm counted the number and intensity-sum in each intensity range, along with three additional quantities: average intensity, ratio of strong/total number, and average intensity of weak positive pixels. The positive pixel algorithm was modified to distinguish between the orange and blue colors. Alterations from the normal “hue value” (0.1 to 0.96) and “color saturation” (0.04 to 0.29), were made for the Sirius Red evaluation. Vasculature and artifacts were excluded from analysis.

### Liver Gene Expression Analysis

Liver gene expression of MCP-1 and MIP-1a was measured by quantitative PCR.

### Liver Cytokine and Chemokine Measurement

Liver IL-1b, MCP-1, and MIP-1 protein levels were quantified using the multiplex ELISA Assay (Meso Scale Discovery, Rockville, Md.).

### Statistical Analysis

Statistical analyses of liver histological scores were performed using Bonferroni Multiple Comparison Test on GraphPad Prism 6 (GraphPad Software Inc., USA). P values <0.05 were considered statistically significant. Results were expressed as mean±SEM. Comparisons were made between Group 2 (Vehicle) and the following groups; Group 3 (LIVRQNAC 1,500 mg/kg), Group 4 (LIVRQNAC 3,000 mg/kg), Group 5 (LIVRQNAC+G, 3,885 mg/kg), and (LRQNAC, 2,469 mg/kg).

### Body and Liver Weight

Feeding the western diet supplemented with fructose (WDF) for 16 weeks elicited significant effects on body weight compared to control fed animals. Prior to administration of test agent, animals fed the WDF were significantly heavier (47.6±0.45 vs. 43.9±1.03 g; p<0.01) compared to animals fed the control diet.

Body weight decreased compared to baseline values in all treatment groups; there were no significant differences in weight loss compared to vehicle (−7.6±0.9, −6.9±1.3, −6.8±1.4, −5.7±1.2, −6.4±1.0, −4.7±1.6 and −3.9±1.5% for control, vehicle, LIVRQNAC (1500 mg/kg), LIVRQNAC (3000 mg/kg), LIVRQNAC+G, LRQNAC, and OCA, respectively; p<0.4992).

Liver weight (% body weight) was significantly higher in vehicle treated animals fed WDF compared to control diet (7.22±0.3 vs. 5.05±0.24%; p<0.0001); however, in animals fed WDF, no significant effects compared to vehicle were noted in any treatment group (7.22±03, 7.14±0.3, 7.19±0.26, 6.69±0.18, 7.02±0.5 and 6.81±0.2 for vehicle, LIVRQNAC (1500 mg/kg), LIVRQNAC (3000 mg/kg), LIVRQNAC+G, LRQNAC, and OCA, respectively; p<0.7450).

### Blood Glucose

Feeding the western diet supplemented with fructose (WDF) for 16 weeks elicited significant effects on glycemia compared to control fed animals. Prior to administration of test agent, animals fed the WDF had significantly lower glucose (160.0±3.01 vs. 218.3±28.6 mg/dL; p<0.0001) compared to animals fed the control diet.

Blood glucose, although higher in control animals at baseline, remained relatively stable during 4 weeks of compound administration. When averaged over the dosing period, there were no significant differences in average blood glucose compared to vehicle for any treatment group (166.0±9.7, 157.1±4.6, 154.6±2.3, 159.4±3.8, 155.5±3.8, 153.6±3.0 and 169.7±6.3 mg/dL for control, vehicle, LIVRQNAC (1500 mg/kg), LIVRQNAC (3000 mg/kg), LIVRQNAC+G, LRQNAC, and OCA, respectively; p<0.1587).

### Liver Triglyceride and Cholesterol

Liver triglyceride and cholesterol content were similarly elevated after WDF feeding compared to vehicle treated animals fed control diet (liver triglyceride p<0.0040; liver cholesterol: p<0.0001). Among animals fed WDF, there were no significant differences in liver triglyceride (p<0.1206) when compared to vehicle for any treatment group. While OCA reduced liver cholesterol content compared to vehicle by 32% (p<0.05), no amino acid composition treatment group affected liver cholesterol as compared to WDF feeding vehicle group.

| | | LIVRQNAC | LIVRQNAC | | | |
|---|---|---|---|---|---|---|
| Liver | Vehicle | 1.5 g/kg | 3.0 g/kg | LIVRQNAC + G | LRQNAC | OCA |
| Triglyceride | 31.49 ± 5.85 | 47.63 ± 1.19 | 47.94 ± 1.37 | 50.57 ± 1.58 | 49.47 ± 1.4 | 49.81 ± 1.63 |
| Cholesterol | 8.37 ± 0.065 | 7.74 ± 0.318 | 7.48 ± 0.697 | 6.42 ± 0.648 | 7.84 ± 0.104 | 5.63 ± 0.495 |

### Liver Histology

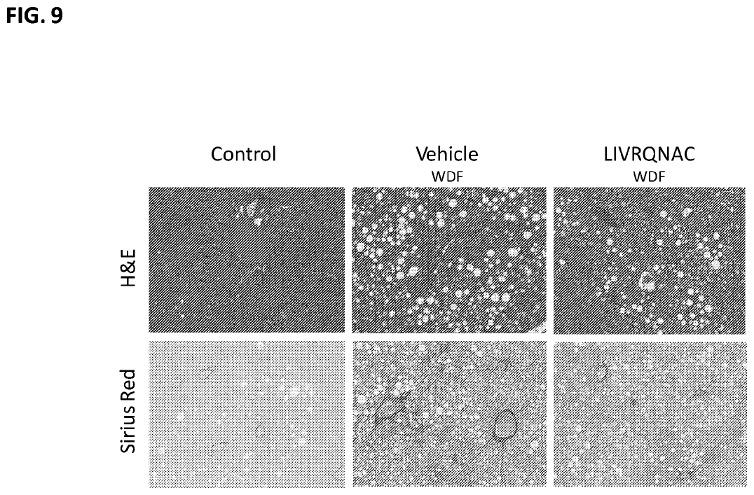

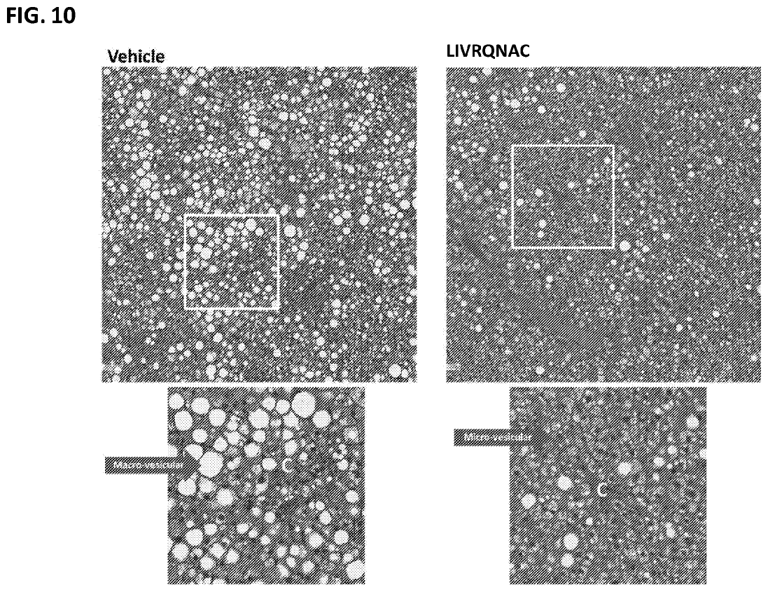

FATZO mice fed with the control diet developed mild steatosis and no inflammation, ballooning, or fibrosis (FIG. 9). FATZO mice fed with the WDF and treated with vehicle developed significant steatosis, mild inflammation, ballooning, and fibrosis. In contrast to predominantly macrovesicular steatosis in the vehicle groups, a mixture of predominantly microvesicular and diminished macrovesicular steatosis was observed in LIVRQNAC, LIVRQNAC+G and LRQNAC groups, as shown in FIG. 10.

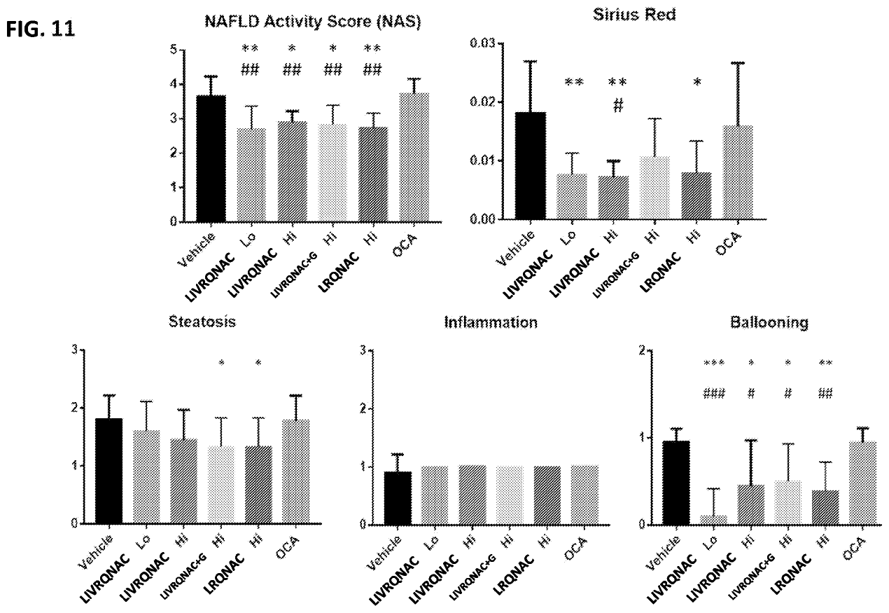

The NAFLD activity score is calculated from histological scoring of steatosis (0-3), inflammation (0-3), and ballooning (0-2) in fixed liver tissues. In WDF fed animals, all amino acid composition treatments produced a significant reduction in the NAS compared to the vehicle treatment group (FIG. 11). LIVRQNAC and amino acid composition treatments reduced liver steatosis as compared to vehicle, although only LIVRQNAC+G and LRQNAC reached statistical significance (p<0.05), while LIVRQNAC did not (LIVRQNAC 3.0 g/kg, p=0.12). All amino acid composition treatments significantly attenuated hepatocyte ballooning, the biomarker of lipotoxicity and cell death. Amino acid composition treatments did not significantly alter liver inflammation. In conclusion, amino acid composition-associated improvement of liver pathology is mainly attributed to attenuation of hepatocyte ballooning.

There was no significant effect of OCA on the NAS score and NAS components compared to vehicle.

| Liver | | LIVRQNAC | LIVRQNAC | | | |
|---|---|---|---|---|---|---|
| Pathology | Vehicle | 1.5 g/kg | 3.0 g/kg | LIVRQNAC + G | LRQNAC | OCA |
| NAS | 3.65 ± 0.183 | 2.70 ± 0.213 | 2.89 ± 0.111 | 2.83 ± 0.186 | 2.72 ± 0.147 | 3.72 ± 0.147 |
| Steatosis | 1.8 ± 0.133 | 1.6 ± 0.163 | 1.44 ± 0.176 | 1.33 ± 0.167 | 1.33 ± 0.167 | 1.78 ± 0.147 |
| Inflammation | 0.9 ± 0.1 | 1.0 ± 0.0 | 1.0 ± 0.0 | 1.0 ± 0.0 | 1.0 ± 0.0 | 1.0 ± 0.0 |
| Ballooning | 0.95 ± 0.05 | 0.1 ± 0.1 | 0.44 ± 0.176 | 0.50 ± 0.144 | 0.39 ± 0.111 | 0.94 ± 0.056 |

Livers from vehicle treated animals demonstrated a mild fibrosis; score of 0.8±0.1. Only livers from animals treated with LIVRQNAC (1500 mg/kg) demonstrated a significant reduction in fibrosis when compared to the vehicle treated group, (0.2±0.1 versus 0.8±0.1, p<0.01), but not with LIVRQNAC (3000 mg/kg), LIVRQNAC+G or LRQNAC. Sirius Red collagen staining demonstrated that all amino acid composition treatments had significantly lower collagen deposition compared to vehicle (LIVRQNAC 1500 mg/kg, p<0.01; LIVRQNAC 3000 mg/kg, p<0.01; LIVRQNAC+G, p=0.09; LRQNAC, p<0.05). OCA did not affect liver fibrosis score or Sirius Red collagen staining area.

| | | LIVRQNAC | LIVRQNAC | | | |
|---|---|---|---|---|---|---|
| Fibrosis | Vehicle | 1.5 g/kg | 3.0 g/kg | LIVRQNAC + G | LRQNAC | OCA |
| Fibrosis | 0.8 ± 0.133 | 0.2 ± 0.133 | 0.44 ± 0.176 | 0.44 ± 0.176 | 0.33 ± 0.167 | 0.67 ± 0.167 |
| Sirius | 1.82 ± 0.279 | 0.77 ± 0.116 | 0.72 ± 0.092 | 0.107 ± 0.218 | 0.79 ± 0.183 | 1.59 ± 0.36 |
| Red | | | | | | |

Consistent with liver triglyceride levels, amino acid composition treatments did not alter liver Oil Red 0 staining area compared to vehicle group. OCA reduced Oil Red 0 staining area (p<0.05).

| | | LIVRQNAC | LIVRQNAC | | | |
|---|---|---|---|---|---|---|
| Oil Red O | Vehicle | 1.5 g/kg | 3.0 g/kg | LIVRQNAC + G | LRQNAC | OCA |
| Oil Red O | 0.32 ± 0.019 | 0.28 ± 0.022 | 0.30 ± 0.022 | 0.26 ± 0.023 | 0.29 ± 0.018 | 0.24 ± 0.021 |
| Triglyceride | 31.49 ± 5.85 | 47.63 ± 1.19 | 47.94 ± 1.37 | 50.57 ± 1.58 | 49.47 ± 1.4 | 49.81 ± 1.63 |

### Liver Gene Expression

MCP-1 (CCL2) and MIP-1a (CCL3) are proinflammatory chemokines that mediate liver inflammation via macrophage and neutrophil recruitment. MCP-1 and MIP-1a are the ligands of CCR2 and CCR5, respectively, which serve the promising therapeutic targets to treat liver fibrosis in NASH. MCP-1 and MIP-1a RNA expression levels in the liver were significantly upregulated in the WDF fed mice as compared to control diet-fed mice, as shown in Tables 74 and 75.

TABLE 74
Fold change in MCP-1 mRNA levels after administration
of amino acid compositions

| | | | LIVRQNAC | LIVRQNAC + G | LRQNAC |
|---|---|---|---|---|---|
| | Ctrl | | (3000 | (3885 | (2469 |
| MCP-1 | diet | Vehicle | mg/kg) | mg/kg) | mg/kg) |
| Mean | 0.1457 | 1.079 | 1.396 | 0.6102 | 0.8777 |
| SEM | 0.0291 | 0.1956 | 0.3414 | 0.09597 | 0.2315 |

TABLE 75
Fold change in MIP-1a mRNA levels after
administration of amino acid compositions

| | | | LIVRQNAC | LIVRQNAC + G | LRQNAC |
|---|---|---|---|---|---|
| | Ctrl | | (3000 | (3885 | (2469 |
| MIP-1a | diet | Vehicle | mg/kg) | mg/kg) | mg/kg) |
| Mean | 0.08328 | 1.194 | 1.67 | 0.814 | 1.514 |
| SEM | 0.02141 | 0.25 | 0.3366 | 0.1029 | 0.525 |

LIVRQNAC and LRQNAC treatments did not significantly alter liver MCP-1 and MIP-1a RNA expression as compared to vehicle group. LIVRQNAC+G treatment resulted in slightly lower liver MCP-1 RNA expression as compared to vehicle group (p=0.054) and LIVRQNAC group (p<0.05). Similarly, LIVRQNAC+G treatment resulted in slightly lower liver MCP-1 RNA expression as compared to vehicle group although the difference was not significant (p=0.19) and LIVRQNAC group (p<0.05).

### Liver Chemokines and Cytokines

Consistent with RNA data, liver MCP-1 and MIP-1a protein levels were elevated in the WDF fed mice as compared to control diet-fed mice, as shown in Tables 76 and 77.

TABLE 76
Mean liver MCP-1 protein levels after administration
of amino acid compositions

| | | | LIVRQNAC | LIVRQNAC + G | LRQNAC |
|---|---|---|---|---|---|
| | Ctrl | | (3000 | (3885 | (2469 |
| MCP-1 | diet | Vehicle | mg/kg) | mg/kg) | mg/kg) |
| Mean | 41.47 | 278.3 | 392 | 221.1 | 247.1 |
| SEM | 7.463 | 61.41 | 83.97 | 36.6 | 75.16 |

TABLE 77
Mean liver MIP-1a protein levels after administration
of amino acid compositions

| | | | LIVRQNAC | LIVRQNAC + G | LRQNAC |
|---|---|---|---|---|---|
| | Ctrl | | (3000 | (3885 | (2469 |
| MIP-1a | diet | Vehicle | mg/kg) | mg/kg) | mg/kg) |
| Mean | 23.16 | 191.8 | 282.9 | 142.8 | 141.1 |
| SEM | 5.429 | 30.03 | 58.88 | 17.04 | 24.36 |

Liver MCP-1 and MIP-1a protein levels were also positively correlated with RNA expression levels, as shown in Tables 78 and 79.

TABLE 78
Correlations between MCP-1 protein and RNA levels
after administration of amino acid compositions

| | | |
|---|---|---|
| Ctrl diet | y = 0.0022x + 0.0542 | R2 = 0.3202 |
| Vehicle | y = 0.0029x + 0.3316 | R2 = 0.7986 |
| LIVRQNAC (3000 mg/kg) | y = 0.0036x + 0.0144 | R2 = 0.7831 |
| LIVRQNAC + G (3885 mg/kg) | y = 0.0018x + 0.2542 | R2 = 0.3988 |
| LRQNAC (2469 mg/kg) | y = 0.0027x + 0.2969 | R2 = 0.6857 |

TABLE 79
Correlations between MIP-1a protein and RNA levels
after administration of amino acid compositions

| | | |
|---|---|---|
| Ctrl diet | y = 0.001x + 0.0593 | R2 = .069 |
| Vehicle | y = 0.0057x + 0.191 | R2 = 0.4202 |
| LIVRQNAC (3000 mg/kg) | y = 0.0051x + 0.2334 | R2 = 0.7887 |
| LIVRQNAC + G (3885 mg/kg) | y = 0.0045x + 0.1817 | R2 = 0.4403 |
| LRQNAC (2469 mg/kg) | y = 0.0064x + 0.1814 | R2 = 0.4875 |

LIVRQNAC and LRQNAC treatments did not significantly alter liver MCP-1 and MIP-1a protein levels as compared to vehicle group. LIVRQNAC+G treatment slightly lowered liver MCP-1 (p=0.095) and MIP-1a (p<0.05) protein levels as compared to LIVRQNAC group. Additionally, liver MCP-1 and MIP-1a protein levels positively correlated, as shown in Table 80.

TABLE 80
Correlations between MCP-1 and MIP-1a protein levels
after administration of amino acid compositions

| | | |
|---|---|---|
| Ctrl diet | y = 0.6803x − 5.0537 | R2 = 0.8744 |
| Vehicle | y = 0.389x + 83.574 | R2 = 0.6325 |
| LIVRQNAC (3000 mg/kg) | y = 0.6615x + 23.609 | R2 = 0.8903 |
| LIVRQNAC + G (3885 mg/kg) | y = 0.4437x + 44.728 | R2 = 0.9082 |
| LRQNAC (2469 mg/kg) | y = 0.3108x + 75.901 | R2 = 0.9241 |

Proinflammatory cytokines IL-1b, IL-6, TNFa, and CXCL1 protein levels in liver were elevated in the WDF fed mice as compared to control diet-fed mice, as shown in Tables 81-84.

TABLE 81
Mean liver IL-1b protein levels after administration
of amino acid compositions

| | | | LIVRQNAC | LIVRQNAC + G | LRQNAC |
|---|---|---|---|---|---|
| | Ctrl | | (3000 | (3885 | (2469 |
| IL-1b | diet | Vehicle | mg/kg) | mg/kg) | mg/kg) |
| Mean | 10.53 | 22.31 | 22.11 | 14.42 | 28.85 |
| SEM | 1.248 | 6.063 | 5.739 | 3.299 | 10.41 |

TABLE 82
Mean liver IL-6 protein levels after administration
of amino acid compositions

| | | | LIVRQNAC | LIVRQNAC + G | LRQNAC |
|---|---|---|---|---|---|
| | Ctrl | | (3000 | (3885 | (2469 |
| IL-6 | diet | Vehicle | mg/kg) | mg/kg) | mg/kg) |
| Mean | 5.838 | 8.452 | 7.298 | 5.77 | 6.71 |
| SEM | 0.3536 | 2.723 | 2.043 | 1.06 | 1.625 |

TABLE 83
Mean liver CXCL1 protein levels after administration
of amino acid compositions

| | | | LIVRQNAC | LIVRQNAC + G | LRQNAC |
|---|---|---|---|---|---|
| | Ctrl | | (3000 | (3885 | (2469 |
| CXCL1 | diet | Vehicle | mg/kg) | mg/kg) | mg/kg) |
| Mean | 41.42 | 105.9 | 119.6 | 77.77 | 84.9 |
| SEM | 2.487 | 13.26 | 26.15 | 8.023 | 10.49 |

TABLE 84
Mean liver TNFα protein levels after
administration of amino acid compositions

| | | | LIVRQNAC | LIVRQNAC + G | LRQNAC |
|---|---|---|---|---|---|
| | Ctrl | | (3000 | (3885 | (2469 |
| TNFa | diet | Vehicle | mg/kg) | mg/kg) | mg/kg) |
| Mean | 1.703 | 3.71 | 4.574 | 2.974 | 4.119 |
| SEM | 0.5641 | 0.4647 | 0.5654 | 0.1513 | 0.8341 |

LIVRQNAC, LIVRQNAC+G, and LRQNAC treatments did not significantly alter IL-1b, IL-6, TNFa, and CXCL1 protein levels as compared to vehicle. Liver TNFα levels were lower by LIVRQNAC+G treatment as compared to LIVRQNAC.

### SUMMARY

Based on clinical observations, WDF-fed FATZO mice gained more body weight that those fed with a control diet. Fed blood glucose levels were comparable between WDF-fed and control diet-fed mice despite of the difference in body weight change. All treatments were well tolerated in FATZO mice. Both WDF-fed and control diet-fed mice lose body weight during the treatment period, which may be due to the stress associated with administration of test articles or vehicle via oral gavage twice a day.

NAS was significantly attenuated in all amino acid composition treatment groups as compared to vehicle, predominantly attributing to ballooning score. Hepatocyte ballooning was significantly reduced in all the amino acid composition treatment groups. Steatosis was significantly reduced in LIVRQNAC+G and LRQNAC treatment groups. LIVRQNAC also lowered steatosis, although the difference was not significant Inflammation was not affected by amino acid composition treatments. Despite the histological improvement in steatosis score in LIVRQNAC+G and LRQNAC treatment groups, liver triglyceride, cholesterol, and Oil-Red 0 staining remained unchanged by amino acid composition treatments. Consistent with the histological and biochemical data, de novo lipogenesis enzymes FASN and ACACA RNA levels were not affected by amino acid composition treatment.

Although liver triglyceride levels were not affected by amino acid composition treatments, the characteristics of hepatocyte steatosis were differed by amino acid composition treatments. Liver of the WDF-fed mice (vehicle group) demonstrated predominantly macrovesicular steatosis. In contrast, macrovesicular steatosis was diminished, and a mixture of microvesicular and macrovesicular steatosis in all amino acid composition treatment groups. The biological meaning and mechanism of amino acid compositions on macro- to microvesicular steatosis phenotypes merit further investigation.

Liver fibrosis score in FATZO model of NAFLD was significantly attenuated by LIVRQNAC treatment at low dose but not at high dose. LIVRQNAC+G and LRQNAC had no effect on fibrosis. Nonetheless, Sirius Red collagen staining demonstrated that LIVRQNAC, LIVRQNAC+G and LRQNAC significantly reduced collagen deposition in the liver.

Consistent with liver inflammation scores, liver RNA and protein levels of the proinflammatory chemokine MCP-1 and MIP-1a and cytokines IL-1b, IL-6, TNFa, and CXCL1 were not significantly affected by amino acid composition treatment. It is of interest to note that LIVRQNAC+G (equivalent to LIVRQNAC plus Glycine) treatment had lower liver MCP-1, MIP-1a, and TNFα as compared to LIVRQNAC.

Increased liver oxidative stress associated with inflammation is observed during NAFLD and NASH. Glutathione (GSH) is a pivotal endogenous anti-oxidant which can counteract reactive oxygen species. Glycine and its direct metabolic precursor, serine, are substrates for GSH biosynthesis. Thus, serine and/or glycine supplementation helps replenish GSH and ameliorates NAFLD and NASH. LIVRQNACG treatment had lower inflammation chemokines and cytokines in the liver, supporting that supplementation of glycine or serine is beneficial in NAFLD and NASH.

In conclusion, all three amino acid compositions (LIVRQNAC, LIVRQNAC+G and LRQNAC) tested in FATZO mice attenuate NAFLD activity scores, hepatocyte ballooning, and fibrosis. These amino acid compositions can be used to treat NASH. Glycine-containing amino acid compositions can further reduce liver inflammation which results in reduced liver fibrosis.

### Example 6. Treatment of Subjects with an Amino Acid Composition

The study described herein features the administration of a composition including amino acids to subjects with type 2 diabetes mellitus (T2DM) and nonalcoholic fatty liver disease (NAFLD). The goal of this pre-IND and IRB approved study was to determine the safety and tolerability of an amino acid composition as well as its impact on the structure and function of human physiology by looking at various markers of insulin sensitivity, glucose and lipid metabolism, after 6 weeks and 12 weeks of administration. The composition included about 1 g of L-leucine, about 0.5 g of L-isoleucine, about 0.5 g of L-valine, about 1.5 g of L-arginine (or 1.81 g of L-arginine HCl), about 2.0 g of L-glutamine, and about 0.15 g of N-acetylcysteine per stick packet, for administration in four stick packs three times per day (e.g., a total of about 72 g per day, or about 24 g three times per day).

In this study, subjects received the amino acid composition three times daily for 12 weeks. Amino acids were provided in powder form to be dissolved in 12 oz. of water. Participants were given the amino acid composition for the 12 week study period.

The primary outcome measure of this study was safety and tolerability. The secondary outcome measures were to examine the impact on human physiology through biomarkers that pertain to metabolism, inflmmation and fibrosis. Assessments were performed at baseline (day 1), at week 6, and at week 12 of the study.

Key criteria for selecting subjects included the following: Men or women aged 18 to 70 years, inclusive; Willing and able to provide written informed consent; History of T2DM or Hemoglobin A1c (HbA1c)≥6.5% and <10% at Screening; Documentation of fatty liver disease by one of the following criteria: a. Prior history of steatosis confirmed within 3 months of Screening by at least one of the following methods: Liver fat by MRI with a PDFF≥8%; Fibroscan with Control Attenuation Parameter ≥300 dB/m; Liver biopsy indicating non-NASH NAFLD steatosis >Grade I. If the patient does not have this documented prior history of steatosis within 3 months of Screening (as noted in 4a), then a liver fat score of ≥10% must be documented at the time of Screening using the following formula:

Predicted percent liver fat=10\hat{ }(−0.805+(0.282*metabolic syndrome [yes=1/no=0])+(0.078*type 2 diabetes [yes=2/no=0])+(0.525*log 10(insulin mU/L))+(0.521*log 10(AST U/L))−(0.454*log 10(AST/ALT))34

Note: insulin, ALT and AST should be measured in a fasted serum sample. Subjects must be on stable exercise, diet and lifestyle routine within 3 months prior to Screening, with no major body weight fluctuations, i.e. subjects should be within ±3% of their body weight over the last 3 months at the time of Screening. Body mass index (BMI)≥32 kg/m2 at Screening. For sites whose MRI equipment cannot accommodate a patient with a BMI of ≥45 kg/m2, an upper limit between 40 to 45 kg/m2 may be applied. Subjects must be on a stable dose of glucose-lowering medication (which can include metformin, sulfonylureas, dipeptidyl peptidase-4 [DPP-4] inhibitors, sodium-glucose co-transporter 2 [SGLT2] inhibitors, or long-acting basal insulin) for at least 3 months before Screening and plan to remain on the same medication without anticipated dose adjustments of their medications for the duration of the study. See Section 8 below for a full list of excluded diabetes related medications. Subjects may be included in the study if they are concurrently treated with anti-hypertensive medications (e.g., beta blockers, hydrochlorothiazide, ACE inhibitors, angiotensin receptor blockers), medications for dyslipidemia (e.g., statins, fibrates), and medication for hypothyroidism (e.g., levothyroxine), so long as they have been on stable doses and regimen of these medications for at least 3 months before Screening and plan to remain on the same medication without anticipated dose adjustments of their medications for the duration of the study. Subjects may be on vitamin supplements (e.g. multivitamins; vitamin E<400 IU/day). However, they must be on stable doses and regimen of these vitamin supplements for at least 3 months before Screening without anticipated dose adjustments for the duration of the study. Female subjects of childbearing potential must have a negative serum pregnancy test at Screening and must agree and use a highly effective method of contraception during heterosexual intercourse during the entire study period and for 30 days following the last dose of study treatment. Childbearing potential refers to those female subjects who have not had a hysterectomy, bilateral oophorectomy, or medically-documented ovarian failure, or women <50 years of age with amenorrhea of any duration.

### Insulin Sensitivity, Glucose and Lipid Metabolism

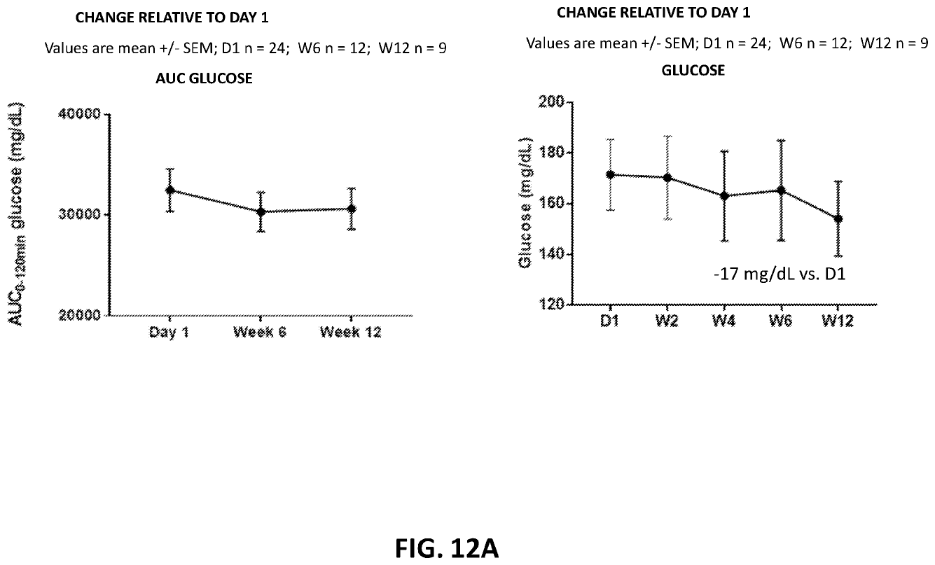

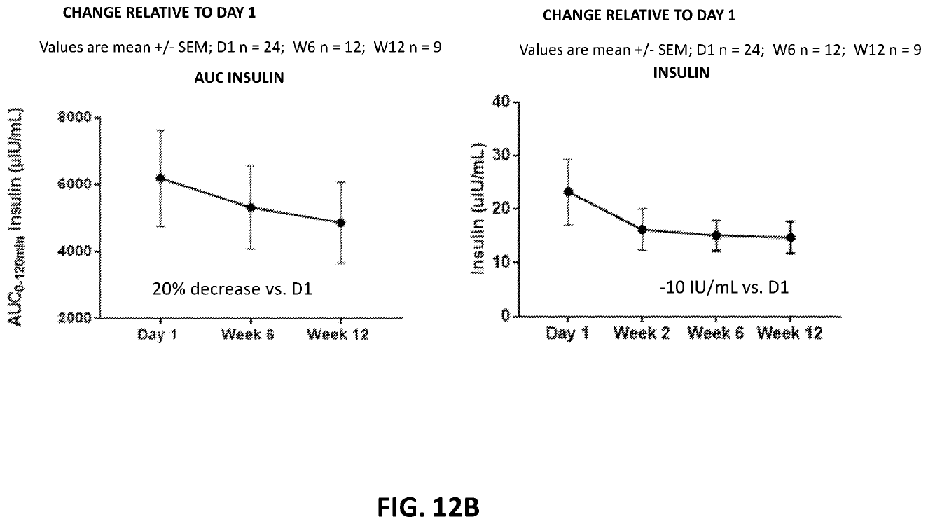

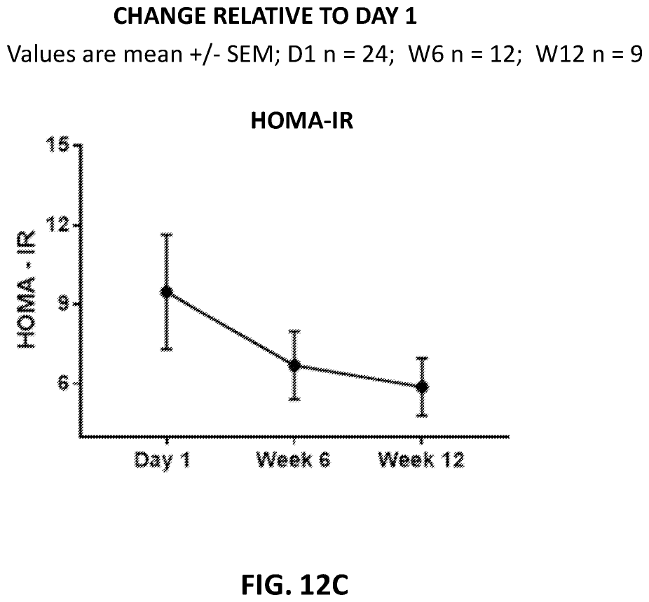

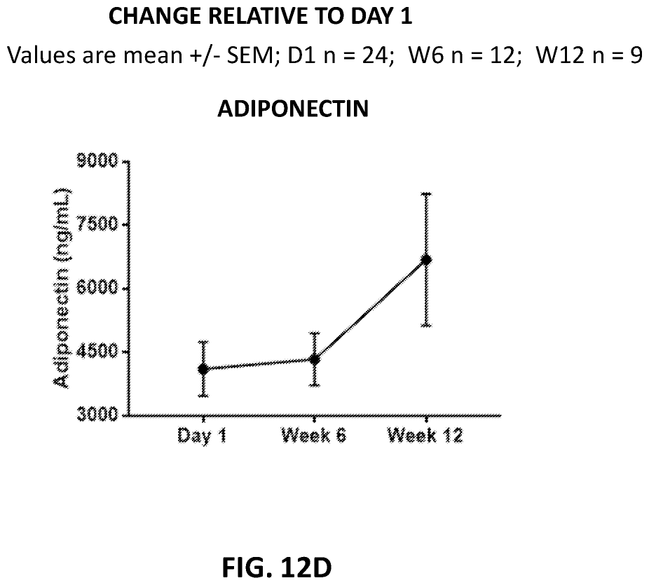

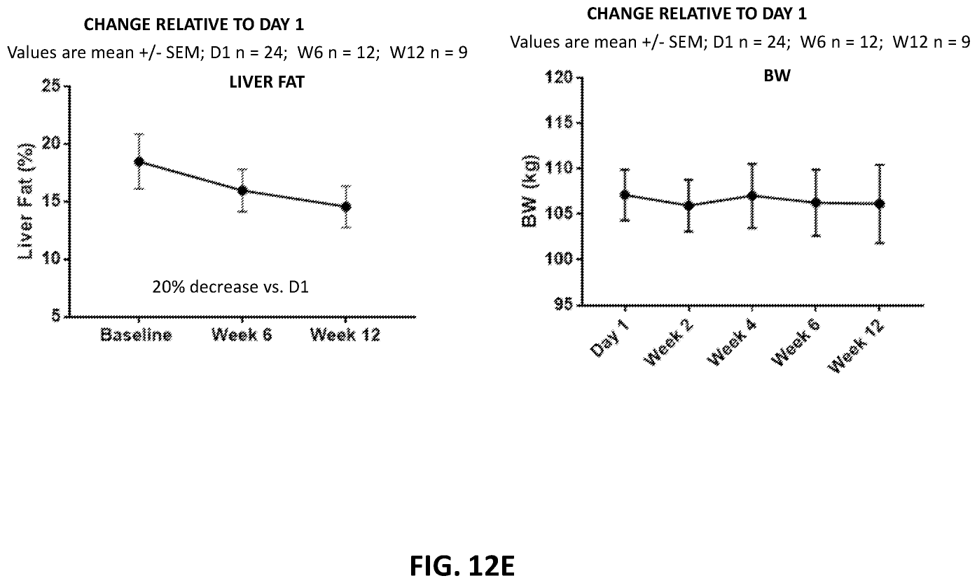

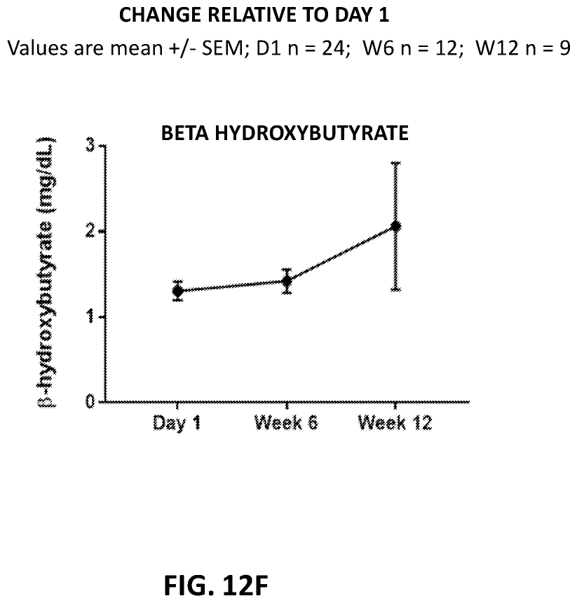

LIVRQNAC improves glucose homeostasis largely by improving insulin sensitivity as shown by reduced fasting glucose (FIG. 12A), fasting insulin (FIG. 12B) and HOMA-IR (FIG. 12C), and an increase at w12 in plasma adiponectin (FIG. 12D), a biomarker of insulin sensitivity. Values are mean+/−SEM. Numbers of subject as indicated.

### Liver Fat, Body Weight and Fatty Acid Oxidation

LIVRQNAC reduces liver fat while increasing fat oxidation, with no change in body weight.

Mean change in liver fat (%) was measured by MRI-PDFF from baseline to Weeks 6 and 12, and mean change in body weight (in kg) from baseline to Weeks 6 and 12 was measured, and mean levels+/−SEM are shown in FIG. 12E. Plasma levels of Beta Hydroxybutyrate were measured, and mean levels+/−SEM are shown in FIG. 12F. FIG. 12E shows no significant change in body weight during the administration period (up to 12 weeks), but a decrease at week 12 in liver fat levels compared to day 1 (baseline) levels. In addition, treatment with the amino acid composition increased levels of beta-hydroxybutyrate (FIG. 12F, mg/dl, mean+/−SEM). Thus, LIVRQNAC reduced liver fat without reducing body weight, while increasing fatty acid oxidation as indicated by increase in beta-hydroxybutyrate.

The findings from this study suggest that the amino acid composition has a favorable safety and tolerability profile and impacts biomarkers for the structure and function of the human body that relate to insulin resistance, improved glucose homeostasis and improved lipid metabolism.

### Example 7. Hepatocyte Model for Measuring Triglyceride Level

Triglyceride (TG) accumulation in the cytoplasm of hepatocytes is a hallmark of NAFLD/NASH. The ability of the amino acids combination L-leucine, L-isoleucine, L-valine, L-arginine, L-glutamine, and N-acetylcysteine (LIVRQNAC) to reduce triglyceride level was assessed using human primary hepatocytes (Lonza, TRL).

### Cell Seeding and Maintenance

Primary hepatocytes from two healthy human donors were seeded on day 0 at density of 3.5e05 cells in 24 well collagen coated plate (Corning) in hepatocyte plating media (William's E medium (Gibco) supplemented with 10% heat-inactivated FBS (Atlanta Bio), 2 mM Glutamax (Gibco), and 0.2% Primocin (InVivoGen) and incubated for 6 hours at 37° C., 5% CO2. After 6 hours, cells were washed twice and incubated overnight at 37° C., 5% CO2. On day 1, cells were washed twice and incubated at 37° C., 5% CO2 for 24 h with hepatocytes defined medium (Corning) supplemented with 2 mM Glutamax (Gibco), and 1× Penicillin/Streptomycin (P/S).

### Amino Acids Pre-Treatment

On day 2, cells were washed twice with DPBS 1× (Gibco) and maintained in:
a. Amino acid-free WEM (US Biologicals) supplemented with 11 mM Glucose (Sigma), 0.272 mM Sodium Pyruvate (Sigma), 1×P/S (Gibco) and containing a defined custom amino acid concentration based on the mean physiological concentrations in blood; or
b. The same media described above with one concentration of defined amino acid compositions LIVRQNAC at 30×.
   Cells were maintained in the defined media (a. and b.) for 24 hours at 37° C., 5% CO2.
   Co-Treatment with Free Fatty Acids and LIVRQNAC

After 24 h pre-treatment, cells were maintained in the same media described above and exposed to free fatty acids (FFA) at 250 uM with a ratio of 2:1 (Oleate:Palmitate) supplemented with TNF-α (Thermofisher) at 1 ng/ml or vehicle. Cells with the free fatty acids mixture (FFAs+TNFα)±LIVRQNAC were incubated for 72 hours at 37° C., 5% CO2 with a media change after the first 24 hours.

Intracellular Triglyceride Level after 72h

After 72 hours, cells were washed once with cold PBS 1× (Gibco), scraped off and collected into 75 ul of standard diluent (Cayman). Collected cells were vortexed for 1 min at a 2500 rpm speed followed by two times sonication (Elmasonic sonicator) for 1 min each. Supernatants were collected for triglyceride analysis after centrifugation at 1000 rpm for 10 min at 4° C. Intracellular triglyceride level was assessed using triglyceride colorimetric assay kit (Cayman) and following manufacturer's recommendations. Data was normalized to the total amount of protein using Bicinchoninic Acid Protein Assay Kit (Sigma).

Table 85 shows the fold change in triglyceride level in primary human hepatocytes from two healthy donors treated with free fatty acids mixture (FFAs+TNFα)+LIVRQNAC (30×) normalized to the FFAs+TNFα baseline (LIVRQNAC 1×). Statistical significance calculated by a T-Test showed that the treatment with LIVRQNAC 30× decreased significantly triglyceride level in the cytoplasm of primary human hepatocytes.

TABLE 85
Changes in triglyceride level for donor 1 and 2
upon administration of the amino acid composition

| | | Triglyceride level relative to Control - Donor 1 | | | | |
|---|---|---|---|---|---|---|
| Amino Acid | Conc. | Mean | Std. | Number | P- | Signifi- |
| Supplement | (X) | log2 | Deviation | of values | value* | cance |
| LIVRQNAC | 30 | −0.36 | 0.23 | 4 | 0.0461 | * |
| LIVRQNAC | 1 | −0.01 | 0.15 | 4 | n/a | n/a |

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

What is claimed is:

1. A method for improving glucose tolerance in a subject, comprising administering to the subject in need thereof an effective amount of a composition comprising:
a) a leucine amino acid entity,
b) an arginine amino acid entity,
c) glutamine amino acid entity; and
d) a N-acetylcysteine (NAC) entity;
provided that:
the glucose tolerance is not associated with a liver condition or disorder, type 2 diabetes, obesity, metabolic syndrome, or a high BMI,
thereby improving glucose tolerance in the subject.

2. A method for reducing insulin resistance in a subject, comprising administering to the subject in need thereof an effective amount of a composition comprising:
a) a leucine amino acid entity,
b) an arginine amino acid entity,
c) glutamine amino acid entity; and
d) a N-acetylcysteine (NAC) entity;
provided that:
the insulin resistance is not associated with a liver condition or disorder, type 2 diabetes, obesity, metabolic syndrome, or a high BMI,
thereby reducing the insulin resistance in the subject.

3. A method of treating insulin resistance in a subject in need thereof, comprising administering to the subject an effective amount of a composition comprising:
a) a leucine amino acid entity,
b) an arginine amino acid entity,
c) glutamine amino acid entity; and
d) NAC-entity;
provided that:
the insulin resistance is not associated with a liver condition or disorder, type 2 diabetes, obesity, metabolic syndrome, or a high BMI,
thereby treating the insulin resistance in the subject.

4. A composition for use in improving glucose tolerance in a subject, comprising an effective amount of a composition comprising:
a) a leucine amino acid entity,
b) an arginine amino acid entity,
c) glutamine amino acid entity; and
d) a N-acetylcysteine (NAC) entity;
provided that:
the glucose tolerance is not associated with a liver condition or disorder, type 2 diabetes, obesity, metabolic syndrome, or a high BMI.

5. A composition for use in reducing insulin resistance in a subject, comprising an effective amount of a composition comprising:
a) a leucine amino acid entity,
b) a arginine amino acid entity,
c) glutamine amino acid entity; and
d) a N-acetylcysteine (NAC) entity;
provided that:
the insulin resistance is not associated with a liver condition or disorder, type 2 diabetes, obesity, metabolic syndrome, or a high BMI.

6. A composition for use in treating insulin resistance in a subject in need thereof, comprising an effective amount of a composition comprising:
a) a leucine amino acid entity,
b) a arginine-amino acid entity,
c) glutamine-amino acid entity; and
d) NAC-entity;
provided that:
the insulin resistance is not associated with a liver condition or disorder, type 2 diabetes, obesity, metabolic syndrome, or a high BMI.

7. The method of any of claims 1-3, or the composition for use of any of claims 4-6, wherein administration of the composition results in an improvement in a metabolic symptom chosen from one, two, three, or more (e.g., all) of decreased free fatty acid, decreased lipid metabolism, increased insulin secretion, or impaired glucose tolerance.

8. The method of any of claim 1-3 or 7, or the composition for use of any of claims 4-7, wherein the subject that has or has been diagnosed with one or both of insulin resistance or impaired glucose tolerance.

9. The method of any of claim 1-3, 7, or 8, or the composition for use of any of claims 4-8, wherein the subject has prediabetes, renal failure, or is pregnant.

10. The method of any of claim 1-3 or 7-9, or the composition for use of any of claims 4-9, wherein the subject has a cardiovascular condition or disorder (e.g., hypertension, dyslipidemia, atherosclerosis, or obstructive sleep apnea), an endocrine condition or disorder (e.g., polycystic ovarian syndrome (PCOS), hyperthyroidism, Cushing's disease, Cushing's syndrome acromegaly, or pheochromocytoma), or a genetic condition or disorder (e.g., Down's syndrome, Turner's syndrome, Klinefelter's syndrome, thalassaemia, haemochromatosis, lipodystrophy, progeria, Huntington's chorea, Friedrich's ataxia, Laurence-Moon-Biedl syndrome, a glycogen storage disease type I, a glycogen storage disease type III, or an inherited mitochondrial disorder).

11. The method of any of claim 1-3 or 7-10, or the composition for use of any of claims 4-10, wherein the subject has a cancer or a dementia.

12. The method of claim 11, or the composition for use of claim 11, wherein the cancer is chosen from: colon cancer, endometrial cancer, pancreatic cancer, renal-cell cancer, or breast cancer.

13. The method of claim 11, or the composition for use of claim 11, wherein the dementia is chosen from: Alzheimer's disease or Lewy body dementia.

14. The method of any of claim 1-3 or 7-13, or the composition for use of any of claims 4-13, wherein the subject has a syndrome of severe insulin resistance (SSIR).

15. The method of any of claim 1-3 or 7-14, wherein the subject has a genetic disorder of insulin resistance.

16. The method of claim 15, or the composition for use of claim 15, or the composition for use of any of claims 4-15, wherein the genetic disorder of insulin resistance is chosen from: Donohue Syndrome, Rabson-Mendenhall Syndrome, or Type A Insulin Resistance.

17. The method of any of claim 1-3 or 7-16, or the composition for use of any of claims 4-16, wherein the method further comprises determining the level of one, two, three, four, five, six, seven, eight, nine, ten, eleven, twelve, or more (e.g. all) of: (a) body weight; (b) BMI; (c) HOMA-IR; (d) glucose utilization in hyperinsulinemic-euglycemic clamp; (e) 2-deoxy glucose uptake in ex vivo skeletal muscle or adipocyte prep; (f) 18FDG PET; (g) adiponectin; (h) retinol binding protein 4 (RBP4); (i) resistin; (j) insulin; (k) glucose; (l) leptin; or (m) adipocyte size.

18. The method of any of claim 1-3 or 7-17, or the composition for use of any of claims 4-17, wherein the composition further comprises one or both of (e) an isoleucine amino acid entity or (f) a valine amino acid entity.

19. The method of any of claim 1-3 or 7-18, or the composition for use of any of claims 4-18, wherein the composition further comprises one, two, three, or more (e.g., all) of: (g) a histidine amino acid entity, (h) a lysine amino acid entity, (i) a phenylalanine amino acid entity, or (j) a threonine amino acid entity.

20. The method of any of claim 1-3 or 7-19, or the composition for use of any of claims 4-19, wherein the total wt. % of (a)-(d) is greater than the total wt. % of one, two, or three of other amino acid entity components, non-amino acid entity protein components (e.g., whey protein), or non-protein components in the composition (e.g., in dry form).

21. The method of any of claim 1-3 or 7-20, or the composition for use of any of claims 4-20, wherein the composition comprises a combination of 18 or fewer amino acid entities.

22. The method of any of claim 1-3 or 7-21, or the composition for use of any of claims 4-21, wherein the composition does not comprise a peptide of more than 20 amino acid residues in length (e.g., whey protein), or if a peptide of more than 20 amino acid residues in length is present, the peptide is present at less than:10 wt. % of the total wt. of non-amino acid entity protein components or total components of the composition (e.g., in dry form).

23. The method of any of claim 1-3 or 7-22, or the composition for use of any of claims 4-22, wherein one, two, three, or more (e.g., all) of methionine, tryptophan, valine, or cysteine is absent from the composition, or if present, are present at less than:10 wt. % of the total wt. of the composition (e.g., in dry form).

24. The method of any of claim 1-3 or 7-23, or the composition for use of any of claims 4-23, wherein one, two, three, four, five, seven, eight, nine, or more (e.g., all) of (a)-(j) is selected from Table 1.

25. The method of any of claim 1-3 or 7-24, or the composition for use of any of claims 4-24, wherein the wt. ratio of the leucine amino acid entity, the arginine amino acid entity, the glutamine amino acid entity, and the NAC-amino acid entity is 1+/−20%:1.5+/−20%:2+/−20%:0.15+/−20%.

26. The method of any of claims 18-25, or the composition for use of any of claims 18-25, wherein the wt. ratio of the leucine amino acid entity, the isoleucine amino acid entity, the valine amino acid entity, the arginine amino acid entity, the glutamine amino acid entity, and the NAC-amino acid entity is 1+/−20%:0.5+/−20%:0.5+/−20%:1.5+/−20%:2+/−20%:0.15+/−20%.

27. The method of any of claim 1-3 or 7-24, or the composition for use of any of claims 4-27, wherein the composition comprises:
a) a leucine amino acid entity chosen from: i) L-leucine or a salt thereof, ii) a dipeptide or salt thereof, or tripeptide or salt thereof, comprising L-leucine, or iii) β-hydroxy-β-methylbutyrate (HMB) or a salt thereof;
b) an arginine amino acid entity chosen from: i) L-arginine or a salt thereof, ii) a dipeptide or salt thereof, or tripeptide or salt thereof, comprising L-arginine, iii) creatine or a salt thereof, or iv) a dipeptide or salt thereof, or tripeptide or salt thereof, comprising creatine;
c) the glutamine amino acid entity is L-glutamine or a salt thereof or a dipeptide or salt thereof, or tripeptide or salt thereof, comprising L-glutamine; and
d) the NAC entity is NAC or a salt thereof or a dipeptide or salt thereof comprising NAC.

28. The method of claim 27, or the composition for use of claim 27, wherein the composition further comprises one or both of: e) L-isoleucine or a salt thereof or a dipeptide or salt thereof, or tripeptide or salt thereof, comprising L-isoleucine; or f) L-valine or a salt thereof or a dipeptide or salt thereof, or tripeptide or salt thereof, comprising L-valine.

29. The method of any of claim 1-3 or 7-28, or the composition for use of any of claims 4-28, wherein the composition further comprises one, two, three, or more (e.g., all) of: (g) L-histidine or a salt thereof or a dipeptide or salt thereof, or tripeptide or salt thereof, comprising L-histidine; (h) L-lysine or a salt thereof or a dipeptide or salt thereof, or tripeptide or salt thereof, comprising L-lysine; (i) L-phenylalanine or a salt thereof or a dipeptide or salt thereof, or tripeptide or salt thereof, comprising L-phenylalanine; or (j) L-threonine or a salt thereof or a dipeptide or salt thereof, or tripeptide or salt thereof, comprising L-threonine.

30. The method of any of claim 1-3 or 7-29, or the composition for use of any of claims 4-29, wherein the composition comprises:
a) the leucine amino acid entity is L-leucine or a salt thereof; b) the arginine amino acid entity is L-arginine or a salt thereof; c) the glutamine amino acid entity is L-glutamine or a salt thereof; and d) the NAC entity is NAC or a salt thereof.

31. The method of any of claim 1-3 or 7-30, or the composition for use of any of claims 4-30, wherein the composition is formulated with a pharmaceutically acceptable carrier.

32. The method of any of claim 1-3 or 7-30, or the composition for use of any of claims 4-13, wherein the composition is formulated as a dietary composition.
